SEQUENCE LISTING (32 sequences)

SEQ ID NO: 1 - protein, 6 aa, Homo sapiens
  misc_feature (1)..(1): Xaa at position 1 could be either His or Ser
XGSXDX

SEQ ID NO: 2 - protein, 12 aa, Homo sapiens
  misc_feature (2)..(2): Xaa at position 2 could be either Ser or Thr
QXYXXXXXXXXX

SEQ ID NO: 3 - protein, 17 aa, Homo sapiens
FIRYDGSNKYYADSVKG

SEQ ID NO: 4 - protein, 7 aa, Homo sapiens
  misc_feature (1)..(1): Xaa at position 1 could be either Gly or Tyr
XNXXRPS

SEQ ID NO: 5 - protein, 9 aa, Homo sapiens
  misc_feature (5)..(5): Xaa represents either Ser or Glu
FTFSXYGMH

SEQ ID NO: 6 - protein, 13 aa, Homo sapiens
  misc_feature (1)..(1): Xaa at position 1 could be either Ser or Thr
XGXXSNIXXXXVX

SEQ ID NO: 7 - protein, 115 aa, Homo sapiens
  misc_feature (6)..(6): Xaa at position 6 could be either Gln or Glu
QVQLVXSGGGVVQPGXSLRLSCAASGFTFSXYGMHWVRQAPGKGLEWVAFIRYDGSNKYYADSBKGRFTISRDNSKNTLYLQMXSLRAEDTAVYYCXXXGSXDXWGQGTMVTVSS

SEQ ID NO: 8 - protein, 112 aa, Homo sapiens
  misc_feature (1)..(1): Xaa at position 1 could be either Ser or Gln
XXVLTQPPSVSGXPGQRVTISCXGXXSNIXXXXVXWYQQLPGTAPKLLIYXNXXRPSGVPDRFSGSKSGTSASLAITGXQAEDEADYYCQXYXXXXXXXXXFGTGTKVTVLG

SEQ ID NO: 9 - protein, 6 aa, Homo sapiens
  misc_feature (2)..(2): Xaa at position 2 could be either Gly, Val, Cys or His
HXXXXX

SEQ ID NO: 10 - protein, 12 aa, Homo sapiens
  misc_feature (4)..(4): Xaa at position 4 could be either Asp or Ser
QSYXXXTHPALL

SEQ ID NO: 11 - protein, 17 aa, Homo sapiens
  misc_feature (1)..(1): Xaa at position 1 could be either Phe, Thr or Tyr
XIXYXXSXKXYADSVKG

SEQ ID NO: 12 - protein, 7 aa, Homo sapiens
  misc_feature (1)..(1): Xaa at position 1 could be either Gly, Tyr, Ser, Thr, Asn or Gln
XNDQRPS

SEQ ID NO: 13 - protein, 9 aa, Homo sapiens
  misc_feature (4)..(5): Xaa at position 4 and 5 represents any amino acid
FTFXXXXMH

SEQ ID NO: 14 - protein, 13 aa, Homo sapiens
  misc_feature (9)..(9): Xaa at position 9 could be either Ser, Cys, Arg, Asn, Asp or Thr
SGGRSNIGXXXVK

SEQ ID NO: 15 - protein, 114 aa, Homo sapiens
  misc_feature (5)..(5): Xaa at position 5 could be either Gln or Glu
QVQVXSGGGVVQPGRSLRLSCAASGFTFSXYGMHWVRQAPGKGLEWVAFIRYDGSNKYYADSVKGRFTISRDNSKNTLYLQMXSLRAEDTAVYYCKTHGSHDNWGQGTMVTVSS

SEQ ID NO: 16 - protein, 112 aa, Homo sapiens
  misc_feature (1)..(1): Xaa at position 1  could be either Ser or Gln
XXVLTQPPSVSGXPGQRVTISCSGXRSNIGSNTVKWYQQLPGTAPKLLIYXNDQRPSGVPDRFSGSKSGTSASLAITGXQAEDEADYYCQSYDRXTHPALLFGTGTKVTVLG

SEQ ID NO: 17 - protein, 6 aa, Homo sapiens
HGSHDN

SEQ ID NO: 18 - protein, 12 aa, Homo sapiens
QSYDRGTHPALL

SEQ ID NO: 19 - protein, 17 aa, Homo sapiens
FIRYDGSNKYYADSVKG

SEQ ID NO: 20 - protein, 7 aa, Homo sapiens
GNDQRPS

SEQ ID NO: 21 - protein, 9 aa, Homo sapiens
FTFSSYGMH

SEQ ID NO: 22 - protein, 13 aa, Homo sapiens
SGGRSNIGSNTVK

SEQ ID NO: 23 - protein, 115 aa, Homo sapiens
QVQLVQSGGGVVQPGRSLRLSCAASGFTFSSYGMHWVRQAPGKGLEWVAFIRYDGSNKYYADSVKGRFTISRDNSKNTLYLQMKSLRAEDTAVYYCKTHGSHDNWGQGTMVTVSS

SEQ ID NO: 24 - protein, 112 aa, Homo sapiens
SYVLTQPPSVSGTPGQRVTISCSGGRSNIGSNTVKWYQQLPGTAPKLLIYGNDQRPSGVPDRFSGSKSGTSASLAITGVQAEDEADYYCQSYDRGTHPALLFGTGTKVTVLG

SEQ ID NO: 25 - protein, 6 aa, Homo sapiens
HGSHDN

SEQ ID NO: 26 - protein, 12 aa, Homo sapiens
QSYDRYTHPALL

SEQ ID NO: 27 - protein, 17 aa, Homo sapiens
FIRYDGSNKYYADSVKG

SEQ ID NO: 28 - protein, 7 aa, Homo sapiens
YNDQRPS

SEQ ID NO: 29 - protein, 9 aa, Homo sapiens
FTFSSYGMH

SEQ ID NO: 30 - protein, 13 aa, Homo sapiens
SGSRSNIGSNTVK

SEQ ID NO: 31 - protein, 115 aa, Homo sapiens [sequence omitted]

SEQ ID NO: 32 - protein, 112 aa, Homo sapiens
QSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLG

(19) United States
(12) Patent Application Publication
(10) Pub. No.: US 2012/0225080 A1
(43) Pub. Date: Sep. 6, 2012

(54) HUMAN ANTIBODIES THAT BIND THE P40 SUBUNIT OF HUMAN IL-12/IL-23 AND USES THEREFOR

(75) Inventors: Matthew W. Hruska, Lindenhurst, IL (US); Susan K. Paulson, Downers Grove, IL (US); Walid Awni, Libertyville, IL (US); Peter Noertersheuser, Dirmstein (DE); Sven Mensing, Mannheim (DE)

(73) Assignee: Abbott Laboratories, Abbott Park, IL (US)

(21) Appl. No.: 13/350,472

(22) Filed: Jan. 13, 2012

Related U.S. Application Data
(63) Continuation-in-part of application No. 12/402,342, filed on Mar. 11, 2009, now Pat. No. 8,178,092.
Application No. 12/402,342 is a continuation-in-part of application No. 12/881,902, filed on Sep. 14, 2010.
(60) Provisional application No. 61/433,074, filed on Jan. 14, 2011.
(60) Provisional application No. 61/482,130, filed on May 3, 2011.
(60) Provisional application No. 61/069,840, filed on Mar. 18, 2008.
(60) Provisional application No. 61/095,275, filed on Sep. 8, 2008.
(60) Provisional application No. 61/207,904, filed on Feb. 18, 2009.
(60) Provisional application No. 61/242,288, filed on Sep. 14, 2009.
(60) Provisional application No. 61/245,967, filed on Sep. 25, 2009.
(60) Provisional application No. 61/297,623, filed on Jan. 22, 2010.
(60) Provisional application No. 61/360,299, filed on Jun. 30, 2010.

(51) Int. Cl. A61K 39/395; A61P 17/06; C07K 16/24

(52) U.S. Cl. 424/142.1; 530/388.15

(57) ABSTRACT

The invention provides human antibodies that bind to the p40 subunit of human IL-12 and/or IL-23. The invention further provides a method of treating psoriasis in a subject by administering to a subject an antibody that binds to the p40 subunit of IL-12 and/or IL-23.

### RELATED APPLICATIONS

This application claims priority to U.S. Provisional Application No. 61/433,074, filed on Jan. 14, 2011 and U.S. Provisional Application No. 61/482,130, filed on May 3, 2011. This application is also a continuation-in-part of U.S. application Ser. No. 12/881,902, filed on Sep. 14, 2010, which claims priority to U.S. Provisional Application No. 61/242,288, filed on Sep. 14, 2009, U.S. Provisional Application No. 61/245,967, filed on Sep. 25, 2009, U.S. Provisional Application No. 61/297,623, filed on Jan. 22, 2010, and U.S. Provisional Application No. 61/360,299, filed on Jun. 30, 2010. This application is also a continuation-in-part of U.S. application Ser. No. 12/402,342, filed on Mar. 11, 2009, which claims priority to U.S. Provisional Application No. 61/069,840, filed on Mar. 18, 2008, U.S. Provisional Application No. 61/095,275, filed on Sep. 8, 2008 and U.S. Provisional Application No. 61/207,904, filed on Feb. 18, 2009. The entire contents of each of the foregoing applications are hereby incorporated herein by reference.

### BACKGROUND OF THE INVENTION

Psoriasis is a T cell-mediated inflammatory disease that is considered to be one of the most common autoimmune diseases, affecting approximately 2% to 3% of adults, though the global prevalence varies widely (Stern R. S., et al., J Investig Dermatol Symp Proc 2004, 9: 136-39; Davidson A and Diamond B. N Engl J Med 2001, 345: 340-50; Langley R. G. B., et al., Ann Rheum Dis 2005, 64(Suppl II): ii18-23). Psoriasis has a major impact on quality of life (de Korte J, et al., J Investig Dermatol Symp Proc 2004, 9: 140-7; Krueger G, et al., Arch Dermatol 2001, 137: 280-4; Finlay A Y and Coles E C, Br J Dermatol 1995, 132: 236-44) and is associated with a number of psychological and psychosocial problems (Kimball A B, et al., Am J Clin Dermatol 2005, 6: 383-92; Russo P A, et al., Australas J Dermatol 2004, 45: 155-9). Many traditional psoriasis therapies have toxic adverse effects; therefore, their long-term use is limited (Lebwohl M. and Ali S., J Am Acad Dermatol 2001, 45: 487-98; Lebwohl M. and Ali S., J Am Acad Dermatol 2001, 45: 649-61). In addition, many patients with psoriasis are dissatisfied with traditional therapies (Stem R S, et al., J Investig Dermatol Symp Proc 2004, 9: 136-39; Finlay A Y and Ortonne J P, J Cutan Med Surg 2004, 8: 310-20); thus, there is a clear need for therapies that are safer and easier to use and that can be prescribed on a long-term basis.

Interleukin-12 (IL-12) and the related cytokine IL-23 are members of the IL-12 superfamily of cytokines that share a common p40 subunit (Anderson E J R, et al., Springer Semin Immunopathol 2006, 27: 425-42). Both cytokines contribute to the development of the type 1T helper cell (Th1) immune response in psoriasis, but each has a unique role (Rosmarin D and Strober B E, J Drugs Dermatol 2005, 4: 318-25; Hong to K, et al., J Immunol 1999, 162: 7480-91; Yawalkar N, et al., J Invest Dermatol 1998, 111: 1053-57). IL-12 primarily stimulates differentiation of Th1 cells and subsequent secretion of interferon-gamma, whereas IL-23 preferentially stimulates differentiation of naïve T cells into effector T helper cells (Th17) that secrete IL-17, a proinflammatory mediator Rosmarin D and Strober B E, J Drugs Dermatol 2005, 4: 318-25; Harrington Le, et al., Nature Immunol 2005, 6: 1123-32; Park H, et al. Nature Immunol 2005, 6: 1132-41). The overexpression of IL-12 p40 and IL-23 p40 messenger RNA in psoriatic skin lesions suggests that the inhibition of IL-12 and IL-23 with a neutralizing antibody to the IL-12/23 p40 subunit protein may offer an effective therapeutic approach for the treatment of psoriasis (Yawalkar N, et al., J Invest Dermatol 1998, 111: 1053-57; Lee E, et al., J Exp Med 2004, 199: 125-30; Shaker O G, et al., Clin Biochem 2006, 39: 119-25; Piskin G, et al., J Immunol 2006, 176: 1908-15). Such therapeutic approaches for the treatment of psoriasis are clearly needed in the art.

### SUMMARY OF THE INVENTION

The present invention provides methods and compositions for treating a disorder in which the activity of the p40 subunit of IL-12 and/or IL-23 is detrimental using an isolated antibody, or antigen-binding portion thereof, that binds the p40 subunit of human IL-12 and/or human IL-23. In certain embodiments, the antibody, or antigen-binding portion thereof, has certain pharmacokinetic properties when administered to subjects, for example subjects having a disorder in which the activity of the p40 subunit of IL-12 and/or IL-23 is detrimental. In one embodiment, the subject has psoriasis, particularly moderate to severe plaque psoriasis. In another embodiment, the subject has rheumatoid arthritis, Crohn's disease, Multiple Sclerosis or psoriatic arthritis. In yet another embodiment, the subject has sarcoidosis, palmo-plantar pustular psoriasis, palmo-plantar pustulosis, severe palmar plantar psoriasis, active ankylosing spondylitis or primary biliary cirrhosis. The invention also provides methods of using the antibodies, or antigen-binding portions thereof, having the particular pharmacokinetic properties in treating subjects having a disorder in which the activity of the p40 subunit of IL-12 and/or IL-23 is detrimental, e.g., psoriasis.

In one aspect, the invention provides an isolated antibody, or antigen-binding portion thereof, which is capable of binding to an epitope of the p40 subunit of IL-12 and/or IL-23, wherein the antibody, or antigen binding portion thereof, when to administered subcutaneously or intravenously to a subject at a dose of about 100 mg or about 200 mg, is capable of exhibiting one or more pharmacokinetic properties selected from the group consisting of: a) a rate of clearance (C_{L}) of about 0.5 to about 1.0 L/day; b) an absorption constant (kₐ) of about 0.4 to about 0.8 L/day; c) a volume of central compartment volume (V_{c}) of about 3.5 to about 8.5 L; d) a second (peripheral compartment) volume (V₂) of about 2.2 to about 4.2 L; e) a rate of clearance from the central compartment to the second compartment (Q) of about 0.6 to about 1.1 L/day; and f) a bioavailability (F1) of about 0.29 to about 0.50.

In one embodiment, the antibody, or antigen binding portion thereof, has 1, 2, 3, 4, 5, or 6 of the pharmacokinetic properties recited above.

In one embodiment, the antibody, or antigen-binding portion thereof, exhibits a rate of clearance (C_{L}) of about 0.5 to about 1.0 L/day. In another embodiment, the antibody, or antigen-binding portion thereof, exhibits a rate of clearance (C_{L}) of about 0.8 L/day.

In another embodiment, the antibody, or antigen-binding portion thereof, exhibits an absorption constant (kₐ) of about 0.4 to about 0.8 L/day. In another embodiment, the antibody, or antigen-binding portion thereof, exhibits an absorption constant (kₐ) of about 0.6 L/day.

In yet another embodiment, the antibody, or antigen-binding portion thereof, exhibits a volume of central compartment volume (V_{c}) of about 3.5 to about 8.5 L. In another embodiment, the antibody, or antigen-binding portion thereof, exhibits a volume of central compartment volume (V_{c}) of about 6.0 L.

In a further embodiment, the antibody, or antigen-binding portion thereof, exhibits a second (peripheral compartment) volume (V₂) of about 2.2 to about 4.2 L. In another embodiment, the antibody, or antigen-binding portion thereof, exhibits a second (peripheral compartment) volume (V₂) of about 3.2 L.

In one embodiment, the antibody, or antigen-binding portion thereof, exhibits a rate of clearance from the central compartment to the second compartment (Q) of about 0.6 to about 1.1 L/day. In another embodiment, the antibody, or antigen-binding portion thereof, exhibits a rate of clearance from the central compartment to the second compartment (Q) of about 0.8 L/day.

In another embodiment, the antibody, or antigen-binding portion thereof, exhibits a bioavailability (F1) of about 0.29 to about 0.50. In another embodiment, the to antibody, or antigen-binding portion thereof, exhibits a bioavailability (F1) of about 0.4.

In one embodiment, the antibody is administered intravenously. In another embodiment, the antibody is administered subcutaneously.

In another embodiment, the antibody is administered once. In a further embodiment, the antibody is administered more than once.

In one embodiment, the antibody, or antigen-binding portion thereof, is administered at a dose of about 100 mg. In another embodiment, the antibody, or antigen-binding portion thereof, is administered at a dose of about 200 mg. In certain embodiments, the antibody, or antigen-binding portion thereof, is administered at a dose of between about 0.1 and about 5.0 mg/kg. In one embodiment, the antibody, or antigen-binding portion thereof, is administered at a dose of about 0.1, about 0.3, 1.0, 3.0 or 5.0 mg/kg.

In one embodiment, the pharmacokinetic properties are determined using a two compartment model.

In another embodiment, the antibody is J695 or an antibody which competes for binding with J695.

In yet another embodiment, the subject is suffering from a disorder in which the activity of the p40 subunit of IL-12 and/or IL-23 is detrimental, e.g., psoriasis.

In another aspect, the invention provides methods for inhibiting the activity of the p40 subunit of IL-12 and/or IL-23 in a subject suffering from a disorder in which the activity of the p40 subunit of IL-12 and/or IL-23 is detrimental, by administering to the subject the isolated antibody or antibody binding portion thereof of the invention as described herein, such that the activity of the p40 subunit of IL-12 and/or IL-23 in the subject is inhibited.

In yet another aspect, the invention provides methods for treating a subject suffering from a disorder in which the activity of the p40 subunit of IL-12 and/or IL-23 is detrimental, by administering to the subject an antibody, or antigen-binding portion thereof, of the invention as described herein, thereby treating the subject.

In one embodiment, the disorder in which the activity of the p40 subunit of IL-12 and/or IL-23 is detrimental is a disorder selected from the group consisting of psoriasis, rheumatoid arthritis, Crohn's disease, Multiple Sclerosis and psoriastic arthritis. In a selected embodiment, the disorder in which the activity of the p40 subunit of IL-12 and/or IL-23 is detrimental is psoriasis, e.g., severe plaque psoriasis. In another to embodiment, the disorder in which the activity of the p40 subunit of IL-12 and/or IL-23 is detrimental is rheumatoid arthritis. In one embodiment, the disorder in which the activity of the p40 subunit of IL-12 and/or IL-23 is detrimental is Crohn's disease. In a further embodiment, the disorder in which the activity of the p40 subunit of IL-12 and/or IL-23 is detrimental is Multiple Sclerosis. In one embodiment, the disorder in which the activity of the p40 subunit of IL-12 and/or IL-23 is detrimental is psoriatic arthritis.

In another embodiment, the disorder in which the activity of the p40 subunit of IL-12 and/or IL-23 is detrimental is sarcoidosis, palmo-plantar pustular psoriasis, palmo-plantar pustulosis, severe palmar plantar psoriasis, active ankylosing spondylitis or primary biliary cirrhosis.

In yet another embodiment, the disorder in which the activity of the p40 subunit of IL-12 and/or IL-23 is detrimental is an autoimmune disease, e.g., an autoimmune disease associated with inflammation, including, without limitation, rheumatoid spondylitis, allergy, autoimmune diabetes, or autoimmune uveitis.

In one embodiment, the disorder in which the activity of the p40 subunit of IL-12 and/or IL-23 is detrimental is a disorder selected from the group consisting of rheumatoid arthritis, osteoarthritis, juvenile chronic arthritis, Lyme arthritis, psoriatic arthritis, reactive arthritis, spondyloarthropathy, systemic lupus erythematosus, Crohn's disease, ulcerative colitis, inflammatory bowel disease, insulin dependent diabetes mellitus, thyroiditis, asthma, allergic diseases, psoriasis, dermatitis scleroderma, atopic dermatitis, graft versus host disease, organ transplant rejection, acute or chronic immune disease associated with organ transplantation, sarcoidosis, atherosclerosis, disseminated intravascular coagulation, Kawasaki's disease, Grave's disease, nephrotic syndrome, chronic fatigue syndrome, Wegener's granulomatosis, Henoch-Schoenlein purpurea, microscopic vasculitis of the kidneys, chronic active hepatitis, uveitis, septic shock, toxic shock syndrome, sepsis syndrome, cachexia, infectious diseases, parasitic diseases, acquired immunodeficiency syndrome, acute transverse myelitis, Huntington's chorea, Parkinson's disease, Alzheimer's disease, stroke, primary biliary cirrhosis, hemolytic anemia, malignancies, heart failure, myocardial infarction, Addison's disease, sporadic, polyglandular deficiency type I and polyglandular deficiency type II, Schmidt's syndrome, adult (acute) respiratory distress syndrome, alopecia, alopecia greata, seronegative arthopathy, arthropathy, Reiter's disease, psoriatic arthropathy, ulcerative colitic arthropathy, enteropathic synovitis, chlamydia, yersinia and salmonella to associated arthropathy, spondyloarthopathy, atheromatous disease/arteriosclerosis, atopic allergy, autoimmune bullous disease, pemphigus vulgaris, pemphigus foliaceus, pemphigoid, linear IgA disease, autoimmune haemolytic anaemia, Coombs positive haemolytic anaemia, acquired pernicious anaemia, juvenile pernicious anaemia, myalgic encephalitis/Royal Free Disease, chronic mucocutaneous candidiasis, giant cell arteritis, primary sclerosing hepatitis, cryptogenic autoimmune hepatitis, Acquired Immunodeficiency Disease Syndrome, Acquired Immunodeficiency Related Diseases, Hepatitis C, common varied immunodeficiency (common variable hypogammaglobulinaemia), dilated cardiomyopathy, female infertility, ovarian failure, premature ovarian failure, fibrotic lung disease, cryptogenic fibrosing alveolitis, post-inflammatory interstitial lung disease, interstitial pneumonitis, connective tissue disease associated interstitial lung disease, mixed connective tissue disease associated lung disease, systemic sclerosis associated interstitial lung disease, rheumatoid arthritis associated interstitial lung disease, systemic lupus erythematosus associated lung disease, dermatomyositis/polymyositis associated lung disease, Sjodgren's disease associated lung disease, ankylosing spondylitis associated lung disease, vasculitic diffuse lung disease, haemosiderosis associated lung disease, drug-induced interstitial lung disease, radiation fibrosis, bronchiolitis obliterans, chronic eosinophilic pneumonia, lymphocytic infiltrative lung disease, postinfectious interstitial lung disease, gouty arthritis, autoimmune hepatitis, type-1 autoimmune hepatitis (classical autoimmune or lupoid hepatitis), type-2 autoimmune hepatitis (anti-LKM antibody hepatitis), autoimmune mediated hypoglycemia, type B insulin resistance with acanthosis nigricans, hypoparathyroidism, acute immune disease associated with organ transplantation, chronic immune disease associated with organ transplantation, osteoarthrosis, primary sclerosing cholangitis, idiopathic leucopenia, autoimmune neutropenia, renal disease NOS, glomerulonephritides, microscopic vasulitis of the kidneys, lyme disease, discoid lupus erythematosus, male infertility idiopathic or NOS, sperm autoimmunity, multiple sclerosis (all subtypes), insulin-dependent diabetes mellitus, sympathetic ophthalmia, pulmonary hypertension secondary to connective tissue disease, Goodpasture's syndrome, pulmonary manifestation of polyarteritis nodosa, acute rheumatic fever, rheumatoid spondylitis, Still's disease, systemic sclerosis, Takayasu's disease/arteritis, autoimmune thrombocytopenia, idiopathic thrombocytopenia, autoimmune thyroid disease, hyperthyroidism, goitrous autoimmune to hypothyroidism (Hashimoto's disease), atrophic autoimmune hypothyroidism, primary myxoedema, phacogenic uveitis, primary vasculitis and vitiligo.

In one embodiment, the methods of the invention further comprise the administration of an additional agent.

In another aspect, the invention provides a pharmaceutical composition comprising an antibody, or antigen-binding portion thereof, which is capable of binding to an epitope of the p40 subunit of IL-12 and/or IL-23, wherein the antibody, or antigen-binding portion thereof, when administered subcutaneously or intravenously to a subject at a dose of about 100 mg or about 200 mg, is capable of exhibiting one or more pharmacokinetic properties selected from the group consisting of: a) a rate of clearance (C_{L}) of about 0.5 to about 1.0 L/day; b) an absorption constant (kₐ) of about 0.4 to about 0.8 L/day; c) a volume of central compartment volume (V_{c}) of about 3.5 to about 8.5 L; d) a second (peripheral compartment) volume (V₂) of about 2.2 to about 4.2 L; e) a rate of clearance from the central compartment to the second compartment (Q) of about 0.6 to about 1.1 L/day; and f) a bioavailability (F1) of about 0.29 to about 0.50.

In one embodiment, the antibody, or antigen binding portion thereof, has 1, 2, 3, 4, 5, or 6 of the foregoing pharmacokinetic properties.

In one embodiment, the composition is administered intravenously. In another embodiment, the composition is administered subcutaneously.

In one embodiment, the composition is administered once. In another embodiment, the composition is administered more than once.

In one embodiment, the composition is administered at a dose of about 100 mg. In another embodiment, the composition is administered at a dose of about 200 mg.

In another embodiment, the pharmacokinetic properties are determined using a two compartment model.

In one embodiment, the subject is suffering from a disorder in which the activity of the p40 subunit of IL-12 and/or IL-23 is detrimental, e.g., psoriasis.

In another aspect, the invention provides methods of treating a disorder in which the activity of IL-12 and/or IL-23 is detrimental comprising subcutaneously or intravenously administering to the subject a pharmaceutical composition comprising an antibody, or antigen-binding portion thereof, which is capable of binding to an epitope of the p40 subunit of IL-12 and/or IL-23, at a dose of about 100 mg or about 200 mg, to wherein at least one pharmacokinetic characteristic selected from the group consisting of: a) a rate of clearance (C_{L}) of about 0.5 to about 1.0 L/day; b) an absorption constant (kₐ) of about 0.4 to about 0.8 L/day; c) a volume of central compartment volume (V_{c}) of about 3.5 to about 8.5 L; d) a second (peripheral compartment) volume (V₂) of about 2.2 to about 4.2 L; e) a rate of clearance from the central compartment to the second compartment (Q) of about 0.6 to about 1.1 L/day; and f) a bioavailability (F1) of about 0.29 to about 0.50 is achieved following administration of the antibody, or antigen-binding portion thereof to the subject.

In one embodiment, 1, 2, 3, 4, 5, or 6 of the foregoing pharmacokinetic properties are achieved following administration of the antibody, or antigen-binding portion thereof, to the subject.

In one embodiment, the composition is administered intravenously. In another embodiment, the composition is administered subcutaneously.

In one embodiment, the composition is administered once. In another embodiment, the composition is administered more than once.

In one embodiment, the composition is administered at a dose of about 100 mg. In another embodiment, the composition is administered at a dose of about 200 mg.

In one embodiment, the disorder in which the activity of the p40 subunit of IL-12 and/or IL-23 is detrimental is psoriasis. In another embodiment, the psoriasis is moderate to severe plaque psoriasis.

In one embodiment, the disorder in which the activity of the p40 subunit of IL-12 and/or IL-23 is detrimental is a disorder selected from the group consisting of psoriasis, rheumatoid arthritis, Crohn's disease, Multiple Sclerosis and psoriastic arthritis. In one embodiment, the disorder in which the activity of the p40 subunit of IL-12 and/or IL-23 is detrimental is psoriasis. In one embodiment, the disorder in which the activity of the p40 subunit of IL-12 and/or IL-23 is detrimental is rheumatoid arthritis. In one embodiment, the disorder in which the activity of the p40 subunit of IL-12 and/or IL-23 is detrimental is Crohn's disease. In one embodiment, the disorder in which the activity of the p40 subunit of IL-12 and/or IL-23 is detrimental is Multiple Sclerosis. In one embodiment, the disorder in which the activity of the p40 subunit of IL-12 and/or IL-23 is detrimental is psoriatic arthritis.

In one embodiment, the disorder in which the activity of the p40 subunit of IL-12 and/or IL-23 is detrimental is sarcoidosis, palmo-plantar pustular psoriasis, palmo-plantar pustulosis, severe palmar plantar psoriasis, active ankylosing spondylitis or primary biliary cirrhosis.

In one embodiment, the disorder in which the activity of the p40 subunit of IL-12 and/or IL-23 is detrimental is an autoimmune disease, e.g., an autoimmune disease associated with inflammation, including, without limitation, rheumatoid spondylitis, allergy, autoimmune diabetes, or autoimmune uveitis.

In one embodiment, the disorder in which the activity of the p40 subunit of IL-12 and/or IL-23 is detrimental is a disorder selected from the group consisting of rheumatoid arthritis, osteoarthritis, juvenile chronic arthritis, Lyme arthritis, psoriatic arthritis, reactive arthritis, spondyloarthropathy, systemic lupus erythematosus, Crohn's disease, ulcerative colitis, inflammatory bowel disease, insulin dependent diabetes mellitus, thyroiditis, asthma, allergic diseases, psoriasis, dermatitis scleroderma, atopic dermatitis, graft versus host disease, organ transplant rejection, acute or chronic immune disease associated with organ transplantation, sarcoidosis, atherosclerosis, disseminated intravascular coagulation, Kawasaki's disease, Grave's disease, nephrotic syndrome, chronic fatigue syndrome, Wegener's granulomatosis, Henoch-Schoenlein purpurea, microscopic vasculitis of the kidneys, chronic active hepatitis, uveitis, septic shock, toxic shock syndrome, sepsis syndrome, cachexia, infectious diseases, parasitic diseases, acquired immunodeficiency syndrome, acute transverse myelitis, Huntington's chorea, Parkinson's disease, Alzheimer's disease, stroke, primary biliary cirrhosis, hemolytic anemia, malignancies, heart failure, myocardial infarction, Addison's disease, sporadic, polyglandular deficiency type I and polyglandular deficiency type II, Schmidt's syndrome, adult (acute) respiratory distress syndrome, alopecia, alopecia greata, seronegative arthopathy, arthropathy, Reiter's disease, psoriatic arthropathy, ulcerative colitic arthropathy, enteropathic synovitis, chlamydia, yersinia and salmonella associated arthropathy, spondyloarthopathy, atheromatous disease/arteriosclerosis, atopic allergy, autoimmune bullous disease, pemphigus vulgaris, pemphigus foliaceus, pemphigoid, linear IgA disease, autoimmune haemolytic anaemia, Coombs positive haemolytic anaemia, acquired pernicious anaemia, juvenile pernicious anaemia, myalgic encephalitis/Royal Free Disease, chronic mucocutaneous candidiasis, giant cell arteritis, primary sclerosing hepatitis, cryptogenic autoimmune hepatitis, Acquired Immunodeficiency Disease Syndrome, Acquired Immunodeficiency Related Diseases, Hepatitis C, common varied immunodeficiency (common variable to hypogammaglobulinaemia), dilated cardiomyopathy, female infertility, ovarian failure, premature ovarian failure, fibrotic lung disease, cryptogenic fibrosing alveolitis, post-inflammatory interstitial lung disease, interstitial pneumonitis, connective tissue disease associated interstitial lung disease, mixed connective tissue disease associated lung disease, systemic sclerosis associated interstitial lung disease, rheumatoid arthritis associated interstitial lung disease, systemic lupus erythematosus associated lung disease, dermatomyositis/polymyositis associated lung disease, Sjodgren's disease associated lung disease, ankylosing spondylitis associated lung disease, vasculitic diffuse lung disease, haemosiderosis associated lung disease, drug-induced interstitial lung disease, radiation fibrosis, bronchiolitis obliterans, chronic eosinophilic pneumonia, lymphocytic infiltrative lung disease, postinfectious interstitial lung disease, gouty arthritis, autoimmune hepatitis, type-1 autoimmune hepatitis (classical autoimmune or lupoid hepatitis), type-2 autoimmune hepatitis (anti-LKM antibody hepatitis), autoimmune mediated hypoglycemia, type B insulin resistance with acanthosis nigricans, hypoparathyroidism, acute immune disease associated with organ transplantation, chronic immune disease associated with organ transplantation, osteoarthrosis, primary sclerosing cholangitis, idiopathic leucopenia, autoimmune neutropenia, renal disease NOS, glomerulonephritides, microscopic vasulitis of the kidneys, lyme disease, discoid lupus erythematosus, male infertility idiopathic or NOS, sperm autoimmunity, multiple sclerosis (all subtypes), insulin-dependent diabetes mellitus, sympathetic ophthalmia, pulmonary hypertension secondary to connective tissue disease, Goodpasture's syndrome, pulmonary manifestation of polyarteritis nodosa, acute rheumatic fever, rheumatoid spondylitis, Still's disease, systemic sclerosis, Takayasu's disease/arteritis, autoimmune thrombocytopenia, idiopathic thrombocytopenia, autoimmune thyroid disease, hyperthyroidism, goitrous autoimmune hypothyroidism (Hashimoto's disease), atrophic autoimmune hypothyroidism, primary myxoedema, phacogenic uveitis, primary vasculitis and vitiligo. In one embodiment, the method further comprises the administration of an additional agent.

In one embodiment, the pharmacokinetic properties are determined using a two compartment model.

In one aspect, the invention provides an isolated antibody, or antigen-binding portion thereof, which is capable of binding to an epitope of the p40 subunit of IL-12 and/or IL-23, wherein the antibody, or antigen binding portion thereof, when to administered subcutaneously or intravenously to a subject at a) a first dose amount of the antibody, or antigen-binding portion thereof, according to a first periodicity of about once every 4 weeks; and b) a second dose amount that is about 40-60% of the first dose amount, according to a second periodicity of about once every 4 weeks, is capable of exhibiting one or more pharmacokinetic properties selected from the group consisting of: a) a rate of clearance (C_{L}) of about 0.5 to about 1.0 L/day; b) an absorption constant (kₐ) of about 0.4 to about 0.8 L/day; c) a volume of central compartment volume (V_{c}) of about 3.5 to about 8.5 L; d) a second (peripheral compartment) volume (V₂) of about 2.2 to about 4.2 L; e) a rate of clearance from the central compartment to the second compartment (Q) of about 0.6 to about 1.1 L/day; and f) a bioavailability (F1) of about 0.29 to about 0.50. In one embodiment, the first dose about is about 200 mg. In one embodiment, the second dose amount is about 100 mg.

In one aspect, the invention provides an isolated antibody, or antigen-binding portion thereof, which is capable of binding to an epitope of the p40 subunit of IL-12 and/or IL-23, wherein the antibody, or antigen binding portion thereof, when administered subcutaneously or intravenously to a subject at a) about 200 mg once every four weeks for two doses; and b) about 100 mg every four weeks thereafter, is capable of exhibiting one or more pharmacokinetic properties selected from the group consisting of: a) a rate of clearance (C_{L}) of about 0.5 to about 1.0 L/day; b) an absorption constant (kₐ) of about 0.4 to about 0.8 L/day; c) a volume of central compartment volume (V_{c}) of about 3.5 to about 8.5 L; d) a second (peripheral compartment) volume (V₂) of about 2.2 to about 4.2 L; e) a rate of clearance from the central compartment to the second compartment (Q) of about 0.6 to about 1.1 L/day; and f) a bioavailability (F1) of about 0.29 to about 0.50.

In a further aspect, the invention provides an isolated antibody, or antigen-binding portion thereof, which is capable of binding to an epitope of the p40 subunit of IL-12 and/or IL-23, wherein the antibody, or antigen binding portion thereof, when administered subcutaneously or intravenously to a subject at a) about 200 mg at weeks 0 and 4; and b) about 100 mg at week 8 and every 4 weeks thereafter, is capable of exhibiting one or more pharmacokinetic properties selected from the group consisting of: a) a rate of clearance (C_{L}) of about 0.5 to about 1.0 L/day; b) an absorption constant (kₐ) of about 0.4 to about 0.8 L/day; c) a volume of central compartment volume (V_{c}) of about 3.5 to about 8.5 L; d) a second (peripheral compartment) volume (V₂) of about 2.2 to about 4.2 L; e) a rate of clearance from the central compartment to the second compartment (Q) of about 0.6 to about 1.1 L/day; and f) a bioavailability (F1) of about 0.29 to about 0.50.

In one aspect, the invention provides a pharmaceutical composition comprising an antibody, or antigen-binding portion thereof, which is capable of binding to an epitope of the p40 subunit of IL-12 and/or IL-23, wherein the pharmaceutical composition, when administered subcutaneously or intravenously to a subject at a) a first dose amount of the antibody, or antigen-binding portion thereof, according to a first periodicity of about once every 4 weeks; and b) a second dose amount that is about 40-60% of the first dose amount, according to a second periodicity of about once every 4 weeks, allows said antibody, or antigen-binding portion thereof, to exhibit one or more pharmacokinetic properties selected from the group consisting of: a) a rate of clearance (C_{L}) of about 0.5 to about 1.0 L/day; b) an absorption constant (kₐ) of about 0.4 to about 0.8 L/day; c) a volume of central compartment volume (V_{c}) of about 3.5 to about 8.5 L; d) a second (peripheral compartment) volume (V₂) of about 2.2 to about 4.2 L; e) a rate of clearance from the central compartment to the second compartment (Q) of about 0.6 to about 1.1 L/day; and f) a bioavailability (F1) of about 0.29 to about 0.50. In one embodiment, the first dose about is about 200 mg. In one embodiment, the second dose amount is about 100 mg.

In one aspect, the invention provides a pharmaceutical composition comprising an antibody, or antigen-binding portion thereof, which is capable of binding to an epitope of the p40 subunit of IL-12 and/or IL-23, wherein the pharmaceutical composition, when administered subcutaneously or intravenously to a subject at a) about 200 mg once every four weeks for two doses; and b) about 100 mg every four weeks thereafter, allows said antibody, or antigen-binding portion thereof, to exhibit one or more pharmacokinetic properties selected from the group consisting of: a) a rate of clearance (C_{L}) of about 0.5 to about 1.0 L/day; b) an absorption constant (kₐ) of about 0.4 to about 0.8 L/day; c) a volume of central compartment volume (V_{c}) of about 3.5 to about 8.5 L; d) a second (peripheral compartment) volume (V₂) of about 2.2 to about 4.2 L; e) a rate of clearance from the central compartment to the second compartment (Q) of about 0.6 to about 1.1 L/day; and f) a bioavailability (F1) of about 0.29 to about 0.50.

In a further aspect, the invention provides a pharmaceutical composition comprising an isolated antibody, or antigen-binding portion thereof, which is capable of to binding to an epitope of the p40 subunit of IL-12 and/or IL-23, wherein the pharmaceutical composition, when administered subcutaneously or intravenously to a subject at a) about 200 mg at weeks 0 and 4; and b) about 100 mg at week 8 and every 4 weeks thereafter, allows said antibody, or antigen binding portion thereof, to exhibit one or more pharmacokinetic properties selected from the group consisting of: a) a rate of clearance (C_{L}) of about 0.5 to about 1.0 L/day; b) an absorption constant (kₐ) of about 0.4 to about 0.8 L/day; c) a volume of central compartment volume (V_{c}) of about 3.5 to about 8.5 L; d) a second (peripheral compartment) volume (V₂) of about 2.2 to about 4.2 L; e) a rate of clearance from the central compartment to the second compartment (Q) of about 0.6 to about 1.1 L/day; and f) a bioavailability (F1) of about 0.29 to about 0.50.

In an embodiment, the invention provides an isolated antibody, or antigen-binding portion thereof, which is capable of binding to an epitope of the p40 subunit of IL-12 and/or IL-23, wherein the antibody, or antigen binding portion thereof, has one or more (e.g., 1, 2, 3, 4, 5, or 6) of pharmacokinetic properties as determined using a two compartment model such as:

a) a rate of clearance (C_{L}) of about 0.5 to about 1.0 L/day;

b) an absorption constant (kₐ) of about 0.4 to about 0.8 L/day;

c) a volume of central compartment volume (V_{c}) of about 3.5 to about 8.5 L;

d) a second (peripheral compartment) volume (V₂) of about 2.2 to about 4.2 L;

e) a rate of clearance from the central compartment to the second compartment (Q) of about 0.6 to about 1.1 L/day; and

f) a bioavailability (F1) of about 0.29 to about 0.50.

In certain embodiments, the isolated antibody, or antigen-binding portion thereof, has one or more of the foregoing pharmacokinetic properties when administered via intravenous injection. In certain embodiments, the isolated antibody, or antigen-binding portion thereof, has one or more of the foregoing pharmacokinetic properties when administered via subcutaneous injection. In certain embodiments, the isolated antibody, or antigen-binding portion thereof, when administered at a dose of between about 0.1 and about 5.0 mg/kg, has one or more of the foregoing pharmacokinetic properties. In certain embodiments, the isolated antibody, or antigen-binding portion thereof, has one or more of the foregoing pharmacokinetic properties when administered to once prior to determining the pharmacokinetic properties. In certain embodiments, the isolated antibody, or antigen-binding portion thereof, has one or more of the foregoing pharmacokinetic properties when administered at a dose of between about 100 mg to about 200 mg. In certain embodiments, the isolated antibody, or antigen-binding portion thereof, has one or more of the foregoing pharmacokinetic properties when administered at a dose of about 100 mg. In certain embodiments, the isolated antibody, or antigen-binding portion thereof, has one or more of the foregoing pharmacokinetic properties when administered at a dose of about 200 mg. In certain embodiments, the isolated antibody, or antigen-binding portion thereof, has one or more of the foregoing pharmacokinetic properties when administered more than once.

In certain embodiments, the isolated antibody, or antigen-binding portion thereof, when administered to a subject as a single intravenous dose, further has one or more pharmacokinetic properties as determined using a single compartment model, such as:

a) a maximum serum concentration (Cₘₐₓ) of about 2 to about 150 μg/mL;

b) an area under the serum concentration-time curve (AUC) of about 140 to about 13,000 μg×hr/mL, when the antibody or antigen binding portion is administered via intravenous injection;

c) a half life of about 81 hours to about 208 hours;

d) a clearance rate of about 33 mL/hour to about 596 mL/hour; and

e) a volume of distribution of about 8 L to about 10 L.

In certain embodiments, the isolated antibody, or antigen-binding portion thereof, when administered to a subject as a single subcutaneous dose, further has one or more pharmacokinetic properties as determined using a single compartment model, the properties such as:

a) a maximum serum concentration (Cₘₐₓ) of about 0.25 to about 14 μg/mL;

b) an area under the serum concentration-time curve (AUC) of about 80 to about 5,000 μg×hr/mL, when the antibody or antigen binding portion is administered via subcutaneous injection;

c) a half life (t_{1/2}) of about 161 hours to about 221 hours;

d) a clearance rate (C_{L}) of about 91 mL/hour to about 229 mL/hour;

e) a volume of distribution (V_{z}) of about 24 L to about 67 L; and

f) a tₘₐₓ of about 66 hours to about 90 hours.

In certain embodiments, the invention includes methods of treatment of psoriasis in a subject by administration of an isolated antibody, or antigen binding portion thereof, of the invention to the subject whereby psoriasis is treated.

In an aspect, the invention provides a method of treating psoriasis in a subject comprising administering to the subject an isolated antibody, or antigen-binding portion thereof, which is capable of binding to an epitope of the p40 subunit of IL-12 and/or IL-23, and has at least one pharmacokinetic property, as determined using a two compartment model, selected from the group consisting of a rate of clearance (C_{L}) of about 0.5 to about 1.0 L/day (e.g., about 0.64 to about 0.92 L/day; about 0.71 to about 0.85 L/day; about 0.779 L/day); an absorption constant (kₐ) of about 0.4 to about 0.8 l/day (e.g., about 0.47 to about 0.75 l/day; about 0.54 to about 0.68 l/day; about 0.614 l/day); volume of central compartment distribution (V_{c}) of about 3.5 to about 8.5 L (e.g., about 4.48 to about 7.60 L; about 5.26 to about 6.82 L; about 6.04 L); second (peripheral compartment) volume (V₂) of about 2.2 to about 4.2 L (e.g., about 2.57 to about 3.79 L; about 2.88 to about 3.48 L; about 3.18 L); rate of clearance from the first compartment to the second compartment (Q) of about 0.6 to about 1.1 L/day (e.g., about 0.6 to about 1.0 L/day; about 0.7 to about 0.9 L/day; about 0.805 L/day); and bioavailability (F1) of about 0.29 to about 0.50 (e.g., about 0.32 to about 0.47; about 0.35 to about 0.43; about 0.392).

In certain embodiments, the pharmacokinetic properties result from administration of a single dose of the antibody, or antigen binding portion thereof. In certain embodiments, the pharmacokinetic properties result from administration of more than one dose (e.g., 2, 3, 4, 5, 6, 7, 8, 9, 10 or more) of the antibody, or antigen binding portion thereof. In certain embodiments, the subject is dosed with the antibody or antigen binding portion thereof such that the antibody is present in the subject at a steady state level.

In certain embodiments, the pharmacokinetic properties are determined based on samples from a single subject. In certain embodiments, the pharmacokinetic properties are determined based on samples of a population, e.g., a mixed population (e.g., healthy and not healthy), a population with psoriasis, or a population with moderate to severe psoriasis.

In one embodiment, the antibody, or antigen-binding portion thereof, has one of to the pharmacokinetic properties above as determined using a two-compartment model. In one embodiment, the antibody has at least any two of the pharmacokinetic properties above as determined using a two-compartment model. In another embodiment, the antibody has at least any three of the pharmacokinetic properties above as determined using a two-compartment model. In one embodiment, the antibody has at least any four of the pharmacokinetic properties above as determined using a two-compartment model. In a further embodiment, the antibody has at least any five of the pharmacokinetic properties above as determined using a two-compartment model. In another embodiment, the antibody has six of the pharmacokinetic properties above as determined using a two-compartment model.

In certain embodiments, the antibody further has one or more pharmacokinetic property, as determined using a single compartment model (e.g., after a single dose), selected from the group consisting of a maximum serum concentration (Cₘₐₓ) of between about 0.15 and about 150 μg/mL, and an area under the serum concentration-time curve (AUC) of between about 80 and about 13,000 μg×hr/mL, is achieved following administration of the antibody, or antigen-binding portion thereof, either subcutaneously or intravenously; a clearance (C_{L}) of between about 30 and about 600 mL/hr, and a volume of distribution (V_{z}) of between about 8 and about 11 L after administration of the antibody, or antigen-binding portion thereof intravenously; and apparent clearance (C_{L}/F) of between about 90 and about 250 mL/hr, and an apparent volume of distribution (V/F) of between about 23 and about 67 L is achieved after administration of the antibody, or antigen-binding portion thereof subcutaneously.

In certain embodiments, the antibody is administered via subcutaneous injection.

In certain embodiments, the antibody is administered via intravenous injection.

In certain embodiments, the dose is about 0.1 to about 5.0 mg/kg (e.g., about 0.1 to about 1.0 mg/kg, about 0.1 to about 2.0 mg/kg, about 0.1 to about 3.0 mg/kg, about 0.1 to about 4.0 mg/kg, about 1.0 to about 2.0 mg/kg, about 1.0 to about 3.0 mg/kg. about 1.0 to about 4.0 mg/kg or about 1.0 to about 5.0 mg/kg) of the antibody, or antigen-binding portion thereof. In another embodiments, the dose is about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9 or 5.0 mg/kg of the antibody, or antigen-binding portion thereof. In certain embodiments, the dose is between about 50 mg to about 250 mg, e.g., to about 100 mg to about 200 mg, about 50 mg, about 100 mg, about 150 mg, about 200 mg, about 250 mg, or any range bracketed by any of the two values. In other embodiments, the dose is about 50 mg, about 60 mg, about 70 mg, about 75 mg, about 80 mg, about 90 mg, about 100 mg, about 110 mg, about 120 mg, about 125 mg, about 130 mg, about 140 mg, about 150 mg, about 160 mg, about 170 mg, about 175 mg, about 180 mg, about 190 mg, about 200 mg, about 210 mg, about 220 mg, about 225 mg, about 230 mg, about 240 mg or about 250 mg of the antibody, or antigen-binding portion thereof.

In one embodiment of the methods for treating psoriasis in a subject, the antibody, or antigen binding portion thereof, used in the methods of the invention is administered more than once. In certain embodiments, the antibody is administered once a week. In certain embodiments, the antibody is administered every other week. In certain embodiments, the antibody is administered every four weeks.

In one embodiment, the antibody, or antigen-binding portion thereof, is capable of binding to the epitope of the p40 subunit when the p40 subunit is bound to the p35 subunit of IL-12. In yet another embodiment, the antibody, or antigen-binding portion thereof, is capable of binding to the epitope of the p40 subunit when the p40 subunit is bound to a p19 subunit. In one embodiment, the antibody, or antigen-binding portion thereof, is capable of binding to the epitope of the p40 subunit when the p40 subunit is bound to the p35 subunit of IL-12 and when the p40 subunit is bound to a p19 subunit.

In one embodiment, the antibody, or antigen binding portion thereof, binds to an epitope of the p40 subunit of IL-12 to which an antibody selected from the group consisting of Y61 and J695 binds.

In another embodiment, the antibody is further capable of binding to a first heterodimer and is also capable of binding to a second heterodimer, wherein the first heterodimer comprises the p40 subunit of IL-12 and the p35 subunit of IL-12, and wherein the second heterodimer comprises the p40 subunit of IL-12 and a p19 subunit.

In a further embodiment, the antibody neutralizes the activity of the first heterodimer. In another embodiment, the antibody neutralizes the activity of the second heterodimer. In yet another embodiment, the antibody neutralizes the activity of the first heterodimer and the second heterodimer.

In certain embodiments, the antibodies, or antigen binding portions thereof, for to use in the methods of the invention (in addition to binding IL-12 and/or IL-23, and having at least one of the pharmacokinetic properties as determined by the two compartment method as set forth above, and optionally having at least one of the pharmacokinetic properties as determined by the single compartment method as set forth above), may include one or more activities. Examples of these activities include activity in an in vitro PHA assay, activity as an inhibitor of IFNγ production, activity in binding IL-12 and/or IL-23 with a particular K_{d}, dissociation from 11-12 and/or IL-23 with a particular K_{off}, or having a particular activity in a receptor binding assay.

In certain embodiments, the antibody, or antigen binding portion thereof, used in the methods of the invention inhibits phytohemagglutinin blast proliferation in an in vitro PHA assay with an IC₅₀ of 1×10⁻⁹M or less (e.g., 1×10⁻¹⁰ M or less, 1×10⁻¹¹M or less, or 5×10⁻¹²M or less) Alternatively, the antibody, or antigen binding portion thereof, inhibits phytohemagglutinin blast proliferation in an in vitro PHA assay with an IC₅₀ of 1×10⁻⁸M or 1×10⁻⁷M.

In certain embodiments, the antibody, or antigen binding portion thereof, inhibits human IFNγ production with an IC₅₀ of 1×10⁻¹⁰ M or less, e.g., 0.1×10⁻¹¹ M or less, 1×10⁻¹² M or less.

In certain embodiments, the antibody, or antigen binding portion thereof, used in the methods of the invention dissociates from the p40 subunit of IL-12 with a K_{d} of 1×10⁻¹⁰ M or less, 1.34×10⁻¹⁰ or less, or 9.74×10⁻¹¹ or less, and/or a k_{off} rate constant of 1×10⁻³ s⁻¹ or less, 1×10⁻⁴ s⁻¹ or less, 1×10⁻⁵ s⁻¹ or less, or 1×10⁻² s⁻¹ or less, as determined by surface plasmon resonance.

In one embodiment, the antibody, or antigen-binding portion thereof, used in the methods of the invention inhibits IL-12 and/or IL-23 binding to its receptor in an IL-12 or IL-23 receptor binding assay (RBA), respectively, with an IC₅₀ of 1×10⁻⁹ M or less. In one embodiment, the antibody, or antigen-binding portion thereof, inhibits IL-12 and/or IL-23 binding to its receptor in an IL-12 or IL-23 receptor binding assay (RBA), respectively, with an IC₅₀ of 1×10⁻¹⁰ M or less. In one embodiment, the antibody, or antigen-binding portion thereof, inhibits IL-12 and/or IL-23 binding to its receptor in an IL-12 or IL-23 receptor binding assay (RBA), respectively, with an IC₅₀ of 1×10⁻¹¹ M or less.

In addition to the properties set forth above, (a) binding to IL-12 and/or IL-23, (b) having at least one of the pharmacokinetic properties as determined using a two compartment model, and (c) optionally having at least one of the pharmacokinetic properties as determined using a one compartment model; or (d) at least one of the inhibition, binding, or (e) dissociation activities, the antibodies for use in the invention can also include specific CDR and/or light and heavy chain sequences such as those provided herein.

In another embodiment, the antibody, or antigen binding portion thereof, used in the methods of the invention has a heavy chain CDR3 comprising the amino acid sequence of SEQ ID NO: 25 and a light chain CDR3 comprising the amino acid sequence of SEQ ID NO: 26;

In a further embodiment, the antibody, or antigen binding portion thereof, used in the methods of the invention has a heavy chain CDR2 comprising the amino acid sequence of SEQ ID NO: 27 and a light chain CDR2 comprising the amino acid sequence of SEQ ID NO: 28.

In one embodiment, the antibody, or antigen binding portion thereof, used in the methods of the invention has a heavy chain CDR1 comprising the amino acid sequence of SEQ ID NO: 29 and a light chain CDR1 comprising the amino acid sequence of SEQ ID NO: 30.

In one embodiment, the isolated antibody, or antigen-binding portion thereof, when administered in the pharmaceutical compositions of the invention as described herein has at least one pharmacokinetic property, as determined using a two compartment model, selected from the group consisting of

a) a rate of clearance (C_{L}) of about 0.5 to about 1.0 L/day (e.g., about 0.64 to about 0.92 L/day; about 0.71 to about 0.85 L/day; about 0.779 L/day);

b) an absorption constant (kₐ) of about 0.4 to about 0.8 L/day (e.g., about 0.47 to about 0.75 L/day; about 0.54 to about 0.68 L/day; about 0.614 L/day);

c) volume of central compartment distribution (V_{c}) of about 3.5 to about 8.5 L (e.g., about 4.48 to about 7.60 L; about 5.26 to about 6.82 L; about 6.04 L);

d) second (peripheral compartment) volume (V₂) of about 2.2 to about 4.2 L (e.g., about 2.57 to about 3.79 L; about 2.88 to about 3.48 L; about 3.18 L);

e) rate of clearance from the first compartment to the second compartment (Q) of about 0.6 to about 1.1 L/day (e.g., about 0.6 to about 1.0 L/day; about 0.7 to about 0.9 L/day; about 0.805 L/day); and

f) bioavailability (F1) of about 0.29 to about 0.50 (e.g., about 0.32 to about 0.47; about 0.35 to about 0.43; about 0.392).

In one embodiment, the isolated antibody, or antigen binding portion thereof, used in the methods and pharmaceutical compositions of the invention is a chimeric antibody, a humanized antibody or a human antibody.

In one embodiment, the antibody, or antigen-binding portion thereof, is administered to a subject in a pharmaceutical composition comprising the antibody, or antigen binding portion thereof, and a pharmaceutically acceptable carrier. The pharmaceutical composition may also comprise an additional agent, such as a therapeutic agent, e.g., budenoside, epidermal growth factor, corticosteroids, cyclosporin, sulfasalazine, aminosalicylates, 6-mercaptopurine, azathioprine, metronidazole, lipoxygenase inhibitors, mesalamine, olsalazine, balsalazide, antioxidants, thromboxane inhibitors, IL-1 receptor antagonists, anti-IL-1β monoclonal antibodies, anti-IL-6 monoclonal antibodies, growth factors, elastase inhibitors, pyridinyl-imidazole compounds, antibodies or agonists of TNF, LT, IL-1, IL-2, IL-6, IL-7, IL-8, IL-15, IL-16, IL-18, EMAP-II, GM-CSF, FGF, and PDGF, antibodies of CD2, CD3, CD4, CD8, CD25, CD28, CD30, CD40, CD45, CD69, CD90 or their ligands, methotrexate, cyclosporin, FK506, rapamycin, mycophenolate mofetil, leflunomide, NSAIDs, ibuprofen, corticosteroids, prednisolone, phosphodiesterase inhibitors, adenosine agonists, antithrombotic agents, complement inhibitors, adrenergic agents, IRAK, NIK, IKK, p38, MAP kinase inhibitors, IL-1β converting enzyme inhibitors, TNFα □converting enzyme inhibitors, T-cell signalling inhibitors, metalloproteinase inhibitors, sulfasalazine, azathioprine, 6-mercaptopurines, angiotensin converting enzyme inhibitors, soluble cytokine receptors, soluble p55 TNF receptor, soluble p75 TNF receptor, sIL-1R1, sIL-1R11, sIL-6R, antiinflammatory cytokines, IL-4, IL-10, IL-11, IL-13 and TGFβ.

In another embodiment, the therapeutic agent in the pharmaceutical composition administered to the subject may be selected from the group consisting of anti-TNF antibodies and antibody fragments thereof, TNFR-Ig constructs, TACE inhibitors, PDE4 inhibitors, corticosteroids, budenoside, dexamethasone, sulfasalazine, 5-aminosalicylic acid, olsalazine, IL-1β converting enzyme inhibitors, IL-1ra, tyrosine kinase inhibitors, 6-mercaptopurines and IL-11.

In another embodiment, the therapeutic agent may be selected from the group consisting of corticosteroids, prednisolone, methylprednisolone, azathioprine, cyclophosphamide, cyclosporine, methotrexate, 4-aminopyridine, tizanidine, interferon-β1a, interferon-β1b, Copolymer 1, hyperbaric oxygen, intravenous immunoglobulin, clabribine, antibodies or agonists of TNF, LT, IL-1, IL-2, IL-6, IL-7, IL-8, IL-15, IL-16, IL-18, EMAP-II, GM-CSF, FGF, PDGF, antibodies to CD2, CD3, CD4, CD8, CD25, CD28, CD30, CD40, CD45, CD69, CD80, CD86, CD90 or their ligands, methotrexate, cyclosporine, FK506, rapamycin, mycophenolate mofetil, leflunomide, NSAIDs, ibuprofen, corticosteroids, prednisolone, phosphodiesterase inhibitors, adenosine agonists, antithrombotic agents, complement inhibitors, adrenergic agents, IRAK, NIK, IKK, p38 or MAP kinase inhibitors, IL-1β converting enzyme inhibitors, TACE inhibitors, T-cell signalling inhibitors, kinase inhibitors, metalloproteinase inhibitors, sulfasalazine, azathioprine, 6-mercaptopurines, angiotensin converting enzyme inhibitors, soluble cytokine receptors, soluble p55 TNF receptor, soluble p75 TNF receptor, sIL-1R1, sIL-1R11, sIL-6R, sIL-13R, anti-P7s, p-selectin glycoprotein ligand (PSGL), antiinflammatory cytokines, IL-4, IL-10, IL-13 and TGFβ.

In preferred embodiments, the subject is a human subject. In one embodiment, the subject is a male subject. In another embodiment, the subject is a female subject. In another embodiment, the subject is between about 15 and about 100 years of age; about 18 and about 93 years of age; about 20 and about 80 years of age; about 30 and about 70 years of age; or about 40 and about 60 years of age. In one embodiment, the subject is about 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 years of age. In another embodiment, the subject is 44 years of age. In yet another embodiment, the subject weighs about 92 kg. In another embodiment, the subject weighs between about 40 and about 210 kg. In another embodiment, the subject weighs between about 50 and 200 kg, about 60 and about 150 kg; or about 75 and about 100 kg. In one embodiment, the subject weighs about 40, 43, 45, 50, 55, 60, 65, 70, 75, 80, 85, 90, 92, 95, 100, 105, 110, 115, 120, 125, 130, 135, 140, 145, 150, 155, 160, 165, 170, 175, 180, 185, 190, 195, 200, 205 or 210 kg.

### BRIEF DESCRIPTION OF THE DRAWINGS

FIG. 1 shows a schematic of the Briakinumab Population Pharmacokinetic model used in Example 9.

FIGS. 2A-D show the (A) individual and (B) population predicted versus observed concentrations and conditional weighted residuals versus (C) predicted concentrations and (D) versus time for the results presented in Example 9.

### DETAILED DESCRIPTION OF THE INVENTION

The present invention provides methods and compositions for treating a disorder in which the activity of the p40 subunit of IL-12 and/or IL-23 is detrimental using an isolated antibody, or antigen-binding portion thereof, that binds the p40 subunit of human IL-12 and/or human IL-23. In certain embodiments, the antibody, or antigen-binding portion thereof, has certain pharmacokinetic properties when administered to subjects, for example subjects having a disorder in which the activity of the p40 subunit of IL-12 and/or IL-23 is detrimental. In one embodiment, the subject has psoriasis, particularly moderate to severe plaque psoriasis. In another embodiment, the subject has rheumatoid arthritis, Crohn's disease, Multiple Sclerosis or psoriatic arthritis. In yet another embodiment, the subject has sarcoidosis, palmo-plantar pustular psoriasis, palmo-plantar pustulosis, severe palmar plantar psoriasis, active ankylosing spondylitis or primary biliary cirrhosis. The invention also provides methods of using the antibodies, or antigen-binding portions thereof, having the particular pharmacokinetic properties in treating subjects having a disorder in which the activity of the p40 subunit of IL-12 and/or IL-23 is detrimental, e.g., psoriasis.

In order that the present invention may be more readily understood, certain terms are first defined.

The term “antibody” includes an immunoglobulin molecule comprised of four polypeptide chains, two heavy (H) chains and two light (L) chains inter-connected by disulfide bonds. Each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one to domain, CL. The VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. In one embodiment, the antibody used in the compositions and methods of the invention is the antibody described in U.S. Pat. No. 6,914,128, the entire contents of which is expressly incorporated by reference herein. In another embodiment, the antibody used in the compositions and methods of the invention is the antibody J695 (Abbott Laboratories).

The term “antigen-binding portion” of an antibody (or “antibody portion”) includes fragments of an antibody that retain the ability to specifically bind to an antigen (e.g., hIL-12). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al. (1993) Proc. Natl. Acad. Sci. USA 90:6444-6448; Poljak, R. J., et al. (1994) Structure 2:1121-1123). Still further, an antibody or antigen-binding portion thereof may be part of a larger immunoadhesion molecules, formed by covalent or non-covalent association of the antibody or antibody portion with one or more other proteins or peptides. Examples of such immunoadhesion molecules include use of the streptavidin core region to make a tetrameric scFv molecule (Kipriyanov, S. M., et al. (1995) Human Antibodies and Hybridomas 6:93-101) and use of a cysteine residue, a marker peptide and a C-terminal polyhistidine tag to make bivalent and biotinylated scFv molecules (Kipriyanov, S. M., et al. (1994) Mol. Immunol. 31:1047-1058). Antibody portions, such as Fab and F(ab′)₂ fragments, can be prepared from whole antibodies using conventional techniques, such as papain or pepsin digestion, respectively, of whole antibodies. Moreover, antibodies, antibody portions and immunoadhesion molecules can be obtained using standard recombinant DNA techniques, as described herein. Preferred antigen binding portions are complete domains or pairs of complete domains.

The phrase “human interleukin 12” (abbreviated herein as hIL-12, or IL-12), as used herein, includes a human cytokine that is secreted primarily by macrophages and dendritic cells. The term includes a heterodimeric protein comprising a 35 kD subunit (p35) and a 40 kD subunit (p40) which are both linked together with a disulfide bridge. The heterodimeric protein is referred to as a “p70 subunit”. The structure of human IL-12 is described further in, for example, Kobayashi, et al. (1989) J. Exp Med. 170:827-845; Seder, et al. (1993) Proc. Natl. Acad. Sci. 90:10188-10192; Ling, et al. (1995) J. Exp Med. 154:116-127; Podlaski, et al. (1992) Arch. Biochem. Biophys. 294:230-237. The term human IL-12 is intended to include recombinant human IL-12 (rh IL-12), which can be prepared by standard recombinant expression methods.

The terms “Kabat numbering”, “Kabat definitions and “Kabat labeling” are used interchangeably herein. These terms, which are recognized in the art, refer to a system of numbering amino acid residues which are more variable (i.e. hypervariable) than other amino acid residues in the heavy and light chain variable regions of an antibody, or an antigen binding portion thereof (Kabat et al. (1971) Ann. NY Acad, Sci. 190:382-391 and, Kabat, E. A., et al. (1991) Sequences of Proteins of Immunological Interest, Fifth Edition, U.S. Department of Health and Human Services, NIH Publication No. 91-3242). For the heavy chain variable region, the hypervariable region ranges from amino acid positions 31 to 35 for CDR1, amino acid positions 50 to 65 for CDR2, and amino to acid positions 95 to 102 for CDR3. For the light chain variable region, the hypervariable region ranges from amino acid positions 24 to 34 for CDR1, amino acid positions 50 to 56 for CDR2, and amino acid positions 89 to 97 for CDR3.

The Kabat numbering is used herein to indicate the positions of amino acid modifications made in antibodies of the invention. For example, the Y61 anti-IL-12 antibody can be mutated from serine (S) to glutamic acid (E) at position 31 of the heavy chain CDR1 (H31S E), or glycine (G) can be mutated to tyrosine (Y) at position 94 of the light chain CDR3 (L94G→Y).

The term “human antibody” includes antibodies having variable and constant regions corresponding to human germline immunoglobulin sequences as described by Kabat et al. (See Kabat, et al. (1991) Sequences of Proteins of Immunological Interest, Fifth Edition, U.S. Department of Health and Human Services, NIH Publication No. 91-3242). The human antibodies of the invention may include amino acid residues not encoded by human germline immunoglobulin sequences (e.g., mutations introduced by random or site-specific mutagenesis in vitro or by somatic mutation in vivo), for example in the CDRs and in particular CDR3. The mutations preferably are introduced using the “selective mutagenesis approach” described herein. The human antibody can have at least one position replaced with an amino acid residue, e.g., an activity enhancing amino acid residue which is not encoded by the human germline immunoglobulin sequence. The human antibody can have up to twenty positions replaced with amino acid residues which are not part of the human germline immunoglobulin sequence. In other embodiments, up to ten, up to five, up to three or up to two positions are replaced. In a preferred embodiment, these replacements are within the CDR regions as described in detail below. However, the term “human antibody”, as used herein, is not intended to include antibodies in which CDR sequences derived from the germline of another mammalian species, such as a mouse, have been grafted onto human framework sequences.

The phrase “recombinant human antibody” includes human antibodies that are prepared, expressed, created or isolated by recombinant means, such as antibodies expressed using a recombinant expression vector transfected into a host cell (described further in Section II, below), antibodies isolated from a recombinant, combinatorial human antibody library (described further in Section III, below), antibodies isolated from an animal (e.g., a mouse) that is transgenic for human immunoglobulin genes (see e.g., Taylor, L. D., et al. (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences (See Kabat, E. A., et al. (1991) Sequences of Proteins of Immunological Interest, Fifth Edition, U.S. Department of Health and Human Services, NIH Publication No. 91-3242). In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo. In certain embodiments, however, such recombinant antibodies are the result of selective mutagenesis approach or backmutation or both.

An “isolated antibody” includes an antibody that is substantially free of other antibodies having different antigenic specificities (e.g., an isolated antibody that specifically binds hIL-12 is substantially free of antibodies that specifically bind antigens other than hIL-12). An isolated antibody that specifically binds hIL-12 may bind IL-12 molecules from other species (discussed in further detail below). Moreover, an isolated antibody may be substantially free of other cellular material and/or chemicals.

A “neutralizing antibody” (or an “antibody that neutralized hIL-12 activity”) includes an antibody whose binding to hIL-12 results in inhibition of the biological activity of hIL-12. This inhibition of the biological activity of hIL-12 can be assessed by measuring one or more indicators of hIL-12 biological activity, such as inhibition of human phytohemagglutinin blast proliferation in a phytohemagglutinin blast proliferation assay (PHA), or inhibition of receptor binding in a human IL-12 receptor binding assay (see Example 3-Interferon-gamma Induction Assay of U.S. Pat. No. 6,914,128, which is expressly incorporated by reference herein). These indicators of hIL-12 biological activity can be assessed by one or more of several standard in vitro or in vivo assays known in the art (see Example 3 of U.S. Pat. No. 6,914,128, which is expressly incorporated by reference herein).

The term “activity” includes activities such as the binding specificity/affinity of an antibody for an antigen, for example, an anti-hIL-12 antibody that binds to an IL-12 antigen and/or the neutralizing potency of an antibody, for example, an anti-hIL-12 antibody whose binding to hIL-12 inhibits the biological activity of hIL-12, e.g. inhibition of PHA blast proliferation, IFNγ production, antigen binding or dissociation, or inhibition of receptor binding in a human IL-12 receptor binding assay (see Example 3 of U.S. Pat. No. 6,914,128, which is expressly incorporated by reference herein).

The phrase “surface plasmon resonance” includes an optical phenomenon that allows for the analysis of real-time biospecific interactions by detection of alterations in protein concentrations within a biosensor matrix, for example using the BIAcore system (Pharmacia Biosensor AB, Uppsala, Sweden and Piscataway, N.J.). For further descriptions, see Example 5 of U.S. Pat. No. 6,914,128 and Jonsson, U., et al. (1993) Ann. Biol. Clin. 51:19-26; Jonsson, U., et al. (1991) Biotechniques 11:620-627; Johnsson, B., et al. (1995) J. Mol. Recognit. 8:125-131; and Johnnson, B., et al. (1991) Anal. Biochem. 198:268-277, the entire contents of each of which is expressly incorporated by reference herein.

The term “K_{off}”, as used herein, is intended to refer to the off rate constant for dissociation of an antibody from the antibody/antigen complex.

The term “K_{d}”, as used herein, is intended to refer to the dissociation constant of a particular antibody-antigen interaction.

The phrase “nucleic acid molecule” includes DNA molecules and RNA molecules. A nucleic acid molecule may be single-stranded or double-stranded, but preferably is double-stranded DNA.

The phrase “isolated nucleic acid molecule”, as used herein in reference to nucleic acids encoding antibodies or antibody portions (e.g., VH, VL, CDR3) that bind hIL-12 including “isolated antibodies”), includes a nucleic acid molecule in which the nucleotide sequences encoding the antibody or antibody portion are free of other nucleotide sequences encoding antibodies or antibody portions that bind antigens other than hIL-12, which other sequences may naturally flank the nucleic acid in human genomic DNA. Thus, for example, an isolated nucleic acid of the invention encoding a VH region of an anti-IL-12 antibody contains no other sequences encoding other VH regions that bind antigens other than IL-12. The phrase “isolated nucleic acid molecule” is also intended to include sequences encoding bivalent, bispecific antibodies, such as diabodies in which VH and VL regions contain no other sequences other than the sequences of the diabody.

The term “Cₘₐₓ” refers to the maximum or peak serum or plasma concentration of an agent observed in a subject after its administration. “Tₘₐₓ” refers to the time it takes from dosing to reach Cₘₐₓ. With bolus intravenous administration, Cₘₐₓ is defined as occurring at the time of injection; therefore, Tₘₐₓ is defined as zero. In one embodiment of the invention, the antibody, or antigen-binding portion thereof, has a Cₘₐₓ of about 1 to about 210 μg/mL. In one embodiment of the invention, the antibody, or antigen-binding portion thereof, has a Cₘₐₓ of about 1 to about 50, about 50 to about 100, about 100 to about 150, about 150 to about 210, about 50 to about 150, or about 75 to about 125 μg/mL. In one embodiment of the invention, the antibody, or antigen-binding portion thereof, has a Cₘₐₓ of about 1, about 2, about 3, about 4, about 5, about 6, about 6.5, about 7, about 8, about 9, about 10, about 20, about 25, about 27, about 28, about 30, about 35, about 40, about 45, about 50, about 55, about 60, about 65, about 70, about 75, about 80, about 85, about 90, about 95, about 100, about 105, about 110, about 115, about 120, about 125, about 130, about 135, about 140, about 145, about 150, about 155, about 160, about 165, about 170, about 175, about 180, about 185, about 190, about 195, about 200, about 200, about 205, or about 210 μg/mL. In another embodiment of the invention, the antibody, or antigen-binding portion thereof, has a Cₘₐₓ of about 0.15 to about 20 μg/mL. In one embodiment of the invention, the antibody, or antigen-binding portion thereof, has a Cₘₐₓ of about 0.15, about 0.2, about 0.25, about 0.3, about 0.35, about 0.4, about 0.5, about 0.6, about 0.7, about 0.8, about 0.9, about 1, about 1.1, about 1.2, about 1.3, about 1.4, about 1.5, about 1.6, about 1.7, about 1.8, about 1.9, about 2, about 2.25, about 2.5, about 2.75, about 2.8, about 2.9, about 3, about 4, about 5, about 6, about 7, about 8, about 9, about 10, about 11, about 12, about 13, about 13.4, about 14, about 15, about 16, about 17, about 18, about 19 or about 20 μg/mL. In one embodiment of the invention, the antibody, or antigen-binding portion thereof, has a Cₘₐₓ of about 6.48 μg/mL. In one embodiment of the invention, the antibody, or antigen-binding portion thereof, has a Cₘₐₓ of about 6.5 μg/mL. In one embodiment, the antibody, or antigen-binding portion thereof, has a Cₘₐₓ within a numerical range for which any of the foregoing recited values can be the upper and/or lower values of the range, e.g., 6-7 μg/mL, are also encompased by the invention. The term “absorption constant” or “kₐ” is the rate of absorption of a drug from its site of application through the body, and is typically only considered if the drug is administered by a route other to than intravenous administration (e.g. subcutaneous administration). If a drug is administered intravenously by single rapid injection, absorption is by-passed. The time for such injections is usually so short compared to other pharmacokinetic processes that it is customary, in one-compartment systems, to consider the peak plasma concentration and the equilibrium distribution to occur simultaneously (see, e.g., Remington's Pharmaceutical Sciences, 16^{th} Edition, Chapter 37. c. 1980 Mack Publishing Company). In one embodiment, the antibody, or antigen-binding portion thereof, of the invention has a kₐ of about 0.4 to about 0.8 L/day. In another embodiment, the antibody, or antigen-binding portion thereof, of the invention has a kₐ of about 0.4, 0.45, 0.5, 0.55, 0.6, 0.61, 0.614, 0.62, 0.65, 0.7, 0.75 or 0.8 L/day. In one embodiment, the antibody, or antigen-binding portion thereof, of the invention has a kₐ of about 0.614 Lday. In one embodiment, the antibody, or antigen-binding portion thereof, has a kₐ within a numerical range for which any of the foregoing recited values can be the upper and/or lower values of the range, e.g., 0.61-0.62 L/day, are also encompassed by the invention.

The term “volume of distribution” or “V_{d}” is used to quantify the distribution of an adminstered drug. In a one compartment model, the body is assumed to behave as though it were a single compartment, that is, as if there were no barriers to the movement of drug within the total body space and the final equilibrium distribution is attained instantaneously. V_{d} is not necessarily the volume of the body or the total body water. The volume V_{d} is a fictive one considered to be equal to fD/Cₚ, where f is the fraction absorbed, D is the dose, and Cp is the plasma concentration, in which it is hypothetically assumed that the concentration is the same throughout the volume and is equal to the plasma concentration. In reality, concentration is not homogenous throughout, but this cannot be determined from Cp alone (which simply averages all inputs and outputs); as long as distribution equilibrium is rapidly achieved, the kinetics as perceived through blood or urine concentrations are the same whether distribution is homogeneous or heterogeneous (see, e.g., Remington's Pharmaceutical Sciences, 16^{th} Edition, Chapter 37. c. 1980 Mack Publishing Company)

The “central compartment” or “Vc”, is used to describe the first volume of distribution of a drug using a two compartment model. In the two compartment model, the body is considered to have two compartments in dynamic equilibrium. The compartment, into which the drug is directly absorbed and from which the drug eliminated, is called compartment 1 or the central compartment. The blood is a part of this compartment, is the transporting and distributing medium, and is the medium actually sampled for chemical and pharmacokinetic analysis (see, e.g., Remington's Pharmaceutical Sciences, 16^{th} Edition, Chapter 37. c. 1980 Mack Publishing Company). In one embodiment, the antibody, or antigen-binding portion thereof, has a Vc of about 3.5 to about 8.5 L. In one embodiment, the antibody, or antigen-binding portion thereof, has a Vc of about 3.5, about 4, about 4.5, about 5, about 5.5, about 6, about 6.04, about 6.5, about 7, about 7.5, about 8, or about 8.5 L. In one embodiment, the antibody, or antigen-binding portion thereof, has a Vc of about 5.1, 5.2, 5.3, 5.4, 5.5, 5.6, 5.7, 5.8, 5.9, 6.0, 6.04, 6.1, 6.2, 6.3, 6.4 or 6.5 L. In one embodiment, the antibody, or antigen-binding portion thereof, has a Vc of about 6.04 L. In one embodiment, the inter-individual variability for Vc is about 13%, e.g., 12.9%.

In one embodiment, the antibody, or antigen-binding portion thereof, has a Vc within a numerical range for which any of the foregoing recited values can be the upper and/or lower values of the range, e.g., 5.8-6.2 L, are also encompassed by the invention.

In one embodiment, the antibody, or antigen-binding portion thereof, has a mean ‘apparent volume of distribution” or “apparent first volume of distribution” Vc/F of about 3.5 to about 8.5 L. In one embodiment, the antibody, or antigen-binding portion thereof, has a mean apparent volume of distribution Vc/F of about 3.5, about 4, about 4.5, about 5, about 5.5, about 6, about 6.04, about 6.5, about 7, about 7.5, about 8, or about 8.5 L. In one embodiment, the antibody, or antigen-binding portion thereof, has a mean apparent volume of distribution Vc/F of about 5.1, 5.2, 5.3, 5.4, 5.5, 5.6, 5.7, 5.8, 5.9, 6.0, 6.04, 6.1, 6.2, 6.3, 6.4 or 6.5 L. In one embodiment, the antibody, or antigen-binding portion thereof, has a mean apparent volume of distribution Vc/F of about 6.04 L. In one embodiment, the antibody, or antigen-binding portion thereof, has a Vc/F within a numerical range for which any of the foregoing recited values can be the upper and/or lower values of the range, e.g., 5.8-6.2 L, are also encompassed by the invention.

The term “Vₚ”, “V₂”, or “peripheral compartment” in a simple two compartment model is closed and communicates with the environment only through the central compartment, being, as it were, peripheral to the events of absorption and elimination (see, e.g., Remington's Pharmaceutical Sciences, 16^{th} Edition, Chapter 37. c. 1980 Mack Publishing Company). In one embodiment, the antibody, or antigen-binding portion thereof, has a V₂ of about 2.2 L to about 4.2 L. In one embodiment, the antibody, or antigen-binding portion thereof, has a V₂ of about 2.2, about 2.3, about 2.4, about 2.5, about 2.6, about 2.7, about 2.8, about 2.9, about 3.0, about 3.1, about 3.18, about 3.2, about 3.3, about 3.4, about 3.5, about 3.6, about 3.7, about 3.8, about 3.9, about 4.0, about 4.1 or about 4.2 L. In one embodiment, the antibody, or antigen-binding portion thereof, has a V₂ of about 3.18 L or 3.2 L. In one embodiment, the antibody, or antigen-binding portion thereof, has a V₂ within a numerical range for which any of the foregoing recited values can be the upper and/or lower values of the range, e.g., 3.0-3.4 L, are also encompassed by the invention.

The term “V_{z}” is the volume of distribution is used with single compartment models. Apparent V_{z} can be defined as V_{z}/F for subcutaneous administration. In one embodiment, the antibody, or antigen-binding portion thereof, has a V_{z} of about 15 to about 200 L. In one embodiment, the antibody, or antigen-binding portion thereof, has a V_{z} of about 24 to about 67 L, about 25 to about 175, about 15 to about 50, about 50 to about 100, about 100 to about 150, or about 150 to about 200 L. In one embodiment, the antibody, or antigen-binding portion thereof, has a V_{z} of about 15, about 20, about 23.9, about 24, about 24.8, about 25, about 30, about 31.8, about 32, about 35, about 40, about 50, about 60, about 66.5, about 67, about 70, about 75, about 80, about 90, about 100, about 110, about 120, about 125, about 130, about 140, about 150, about 160, about 170, about 175, about 180, about 190, or about 200 L. In another embodiment, the antibody, or antigen-binding portion thereof, has a V_{z} of about 1 to about 15 L. In another embodiment, the antibody, or antigen-binding portion thereof, has a V_{z} of about 1, about 2, about 3, about 4, about 5, about 6, about 7, about 8, about 8.5, about 9, about 9.4, about 10, about 10.4, about 10.5, about 11, about 12, about 13, about 14 or about 15 L. In one embodiment, the antibody, or antigen-binding portion thereof, has a V_{z} within a numerical range for which any of the foregoing recited values can be the upper and/or lower values of the range, e.g., 25-35 L, are also encompassed by the invention.

The terms “V_{c}”, “Vₚ”, and “V_{z}” are fictive and defined by kinetic behavior of the drug within the body and not necessarily by identifiable anatomical entities. The movement of drugs within and between compartments is defined e.g., by characteristic first-order rate constants, and the rate of movement of the drug from the first compartment to the second compartment, or in the reverse direction can be calculated in a manner similar to the rate of absorption (see, e.g., Remington's Pharmaceutical Sciences, 16^{th} Edition, Chapter 37. c. 1980 Mack Publishing Company).

The term “Q” is related to the rate in which a drug moves from the first compartment to the second compartment in a two compartment model. In one embodiment, the antibody, or antigen-binding portion thereof, has a Q of about 0.6 to about 1.1 L/day. In another embodiment, the antibody, or antigen-binding portion thereof, has a Q of about 0.6, 0.7, 0.8, 0.805, 0.9, 1.0 or 1.1 L/day. In another embodiment, the antibody, or antigen-binding portion thereof, has a Q of about 0.805 L/day. In one embodiment, the antibody, or antigen-binding portion thereof, has a Q within a numerical range for which any of the foregoing recited values can be the upper and/or lower values of the range, e.g., 0.7-0.9 L/day, are also encompassed by the invention.

The term “Tₘₐₓ” refers to the time at which Cₘₐₓ occurs. In one embodiment, the antibody, or antigen-binding portion thereof, has a Tₘₐₓ of about 50 to about 140 hours or about 65 to about 90 hours. In another embodiment, the antibody, or antigen-binding portion thereof, has a Tₘₐₓ of about 50, 55, 60, 65, 66.7, 70, 75, 80, 82, 85, 90, 95, 100, 105, 110, 115, 120, 125, 130, 135 or 140 hours.

In another embodiment, the median time to Cₘₐₓ is about 30 hours to about 150 hours; about 50 hours to about 100 hours; or about 60 hours to about 90 hours. In another embodiment, the median time to Cₘₐₓ is about 30 hours, about 35 hours, about 36 hours, about 40 hours, about 45 hours, about 50 hours, about 55 hours, about 60 hours, about 65 hours, about 70 hours, about 75 hours, about 80 hours, about 85 hours, about 90 hours, about 95 hours, about 100 hours, about 105 hours, about 110 hours, about 115 hours, about 120 hours, about 125 hours, about 130 hours, about 140 hours, about 144 hours, about 145 hours, or about 150 hours. In one embodiment, the median time to Cₘₐₓ is about 60 hours. In one embodiment, the median time to Cₘₐₓ is about 1 day to about 6 days, about 1.5 to about 5 days, about 2 to about 3 days, or about 2.5 days. In one embodiment, the antibody, or antigen-binding portion thereof, has a Cₘₐₓ within a numerical range for which any of the foregoing recited values can be the upper and/or lower values of the range, e.g., 50-70 hours, or 55-65 hours, are also encompased by the invention.

The term “C_{L}” or “clearance” is the rate that a drug is eliminated from the plasma, typically substantially through the kidney and liver, however, other paths of elimination are also possible depending on the specific characteristics of the drug. In one embodiment, the antibody, or antigen-binding portion thereof, has a C_{L} of about 0.5 to about 1.0 L/day. In another embodiment, the antibody, or antigen-binding portion thereof, has a C_{L} of about 0.5, 0.6, 0.65, 0.7, 0.71, 0.72, 0.73, 0.74, 0.75, 0.76, 0.77, 0.779, 0.78, 0.79, 0.8, 0.85, 0.9 or 1.0 L/day. In another embodiment, the antibody, or antigen-binding portion thereof, has a C_{L} of about 0.779 or 0.78 L/day. In one embodiment, the inter-individual variability for C_{L} is about 9%, e.g., 8.8%. In one embodiment, the C_{L} increases with increasing bodyweight, e.g., increases by approximately 10% per 10 kg change from greater than 75 kg to about 105 kg, and then increases approximately 7.5% when weight is greater than about 105 kg. In another embodiment, the C_{L} increases in the presence of ADA and neutralizing ADA, e.g., increases by about 30% in the presence of ADA and increases by about 66% in the presence of neutralizing ADA.

In another embodiment, the antibody, or antigen-binding portion thereof, has a C_{L} of about 20 to about 2500 mL/hour. In one embodiment, the antibody, or antigen-binding portion thereof, has a C_{L} of about 60 to about 235 mL/hour. In another embodiment, the antibody, or antigen-binding portion thereof, has a C_{L} of about 20 to about 50 mL/hour, about 50 to about 250 mL/hour, about 250 to about 500 mL/hour, about 500 to about 750 mL/hour, about 750 to about 1000 mL/hour, about 1000 to about 1500 mL/hour, about 1500 to about 2000 mL/hour, or about 2000 to about 2500 mL/hour. In another embodiment, the antibody, or antigen-binding portion thereof, has a C_{L} of about 20, 30, 36.2, 33.6, 40, 50, 50.4, 60, 70, 80, 90, 91.1, 100, 110, 120, 130, 140, 150, 160, 170, 180, 183, 185, 190, 200, 225, 229, 230, 250, 300, 350, 400, 450, 500, 596, 600, 700, 800, 900, 950, 1000, 1100, 1200, 1300, 1400, 1500, 1600, 1700, 1800, 1900, 2000, 2100, 2200, 2300, 2400 or 2500 mL/hour. In one embodiment, the antibody, or antigen-binding portion thereof, has a C_{L} within a numerical range for which any of the foregoing recited values can be the upper and/or lower values of the range, e.g., 0.7 and 0.8 L/day, are also encompased by the invention. “Apparent clearance” can be defined as CL/F for subcutaneous administration. In one embodiment, the antibody, or antigen-binding portion thereof, has an apparent clearance or a mean apparent clearance of about 0.5 to about 1.0 L/day. In another embodiment, the antibody, or antigen-binding portion thereof, has an apparent clearance or mean apparent clearance of about 0.5, 0.6, 0.65, 0.7, 0.71, 0.72, 0.73, 0.74, 0.75, 0.76, 0.77, 0.779, 0.78, 0.79, 0.8, 0.85, 0.9 or 1.0 L/day. In another embodiment, the antibody, or antigen-binding portion thereof, has an apparent clearance or a mean apparent clearance to of about 0.779 or 0.78 L/day. In one embodiment, the inter-individual variability is about 9%, e.g., 8.8%. In one embodiment, the antibody, or antigen-binding portion thereof, has an apparent clearance or a mean apparent clearance within a numerical range for which any of the foregoing recited values can be the upper and/or lower values of the range, e.g., 0.7 and 0.8 L/day, are also encompased by the invention.

The term “bioavailability” or “F %” or “F1” refers to a fraction or percent of a dose which is absorbed and enters the systemic circulation after administration of a given dosage form. The dose of the agent may be administered through any route, preferably, via intravenous or subcutaneous injection. In one embodiment, the antibody, or antigen-binding portion thereof, has a bioavailability of about 0.29 to about 0.50. In another embodiment, the antibody, or antigen-binding portion thereof, has a bioavailability of about 0.35 to about 0.45, or about 0.35 to about 0.40. In another embodiment, the antibody, or antigen-binding portion thereof, has a bioavailability of about 0.45 to about 0.55, or about 0.45 to about 0.50. In another embodiment, the antibody, or antigen-binding portion thereof, has a bioavailability of about 0.29, about 0.3, about 0.35, about 0.36, about 0.37, about 0.38, about 0.39, about 0.392, about 0.4, about 0.45, about 0.47, or about 0.50. In another embodiment, the antibody, or antigen-binding portion thereof, has a bioavailability of about 0.392. In another embodiment, the antibody, or antigen-binding portion thereof, has a bioavailability of about 39.2%. In another embodiment, the antibody, or antigen-binding portion thereof, has a bioavailability of about 0.47. In another embodiment, the antibody, or antigen-binding portion thereof, has a bioavailability of about 47%. In one embodiment, the antibody, or antigen-binding portion thereof, has a bioavailability within a numerical range for which any of the foregoing recited values can be the upper and/or lower values of the range, e.g., 0.3 and 0.4, are also encompased by the invention.

“Half life” or “t½” is the amount of time after administration of the drug for half of the dose to clear the subject. In another embodiment, the antibody, or antigen-binding portion thereof, has a half life of about 60 to about 325 hours; 65 to about 290 hours; about 75 to about 250 hours; about 100 to about 200 hours; or about 125 to about 175 hours. In another embodiment, the antibody, or antigen-binding portion thereof, has a half life of about 60, 65, 70, 75, 80, 81.2, 85, 90, 95, 100, 105, 110, 115, 120, 125, 130, 135, 140, 145, 147, 150, 155, 160, 161, 165, 170, 175, 180, 185, 190, 195, 196, 200, 205, 208, 210, 215, 220, 221, 225, 230, 235, 240, 245, 250, 255, 260, 265, 270, 275, 280, 285, 290, 295, 300, 305, 310, 315, 320 or 325 hours. In one embodiment, the antibody, or antigen-binding portion thereof, has a half life within a numerical range for which any of the foregoing recited values can be the upper and/or lower values of the range, e.g., about 75 to about 85 hours, are also encompased by the invention.

“Area under the curve” or “AUC” is used to describe the serum concentration curve in single compartment pharmacokinetic calculations. In one embodiment, the antibody, or antigen-binding portion thereof, has an AUC of about 70 to about 17,000 μg×hr/mL; about 40 to about 5,300; about 100 to about 15,000; about 250 to about 12,500; about 500 to about 1,000, about 600 to about 900; or about 700 to about 800 μg×hr/mL. In another embodiment, the antibody, or antigen-binding portion thereof, has an AUC of about 40, about 50, about 60, about 70, about 80, about 84, about 84.4, about 85, about 90, about 100, about 110, about 120, about 130, about 140, about 146, about 150, about 160, about 170, about 180, about 190, about 200, about 225, about 244, about 250, about 275, about 300, about 325, about 350, about 375, about 400, about 450, about 500, about 550, about 562, about 600, about 650, about 700, about 750, about 800, about 850, about 900, about 950, about 1000, about 1100, about 1200, about 1300, about 1400, about 1500, about 1600, about 1700, about 1800, about 1900, about 2000, about 2250, about 2410, about 2500, about 2750, about 3000, about 3500, about 4000, about 4500, about 4840, about 5000, about 5500, about 6000, about 6500, about 7000, about 7500, about 8000, about 8500, about 9000, about 9500, about 10,000, about 10,500, about 11,000, about 11,500, about 12,000, about 12,500, about 12,700, about 13,000, about 13,500, about 14,000, about 14,500, about 15,000, about 15,500, about 16,000, about 16,500, or about 17,000 μg×hr/mL. In one embodiment, the antibody, or antigen-binding portion thereof, has an area under the curve within a numerical range for which any of the foregoing recited values can be the upper and/or lower values of the range, e.g., 80 to 90 μg×hr/mL, are also encompased by the invention.

In one embodiment, the antibody, or antigen-binding portion thereof, of the invention has a mean trough serum concentration of about 0.4 g/mL to about 0.7 g/mL at steady state, or about 0.457 g/mL to about 0.644 g/mL at steady state. The term “dosing”, as used herein, refers to the administration of a substance (e.g., an anti-IL-12, anti-IL-23 antibody) to achieve a therapeutic objective (e.g., the treatment of psoriasis).

The terms “biweekly dosing regimen”, “biweekly dosing”, and “biweekly to administration”, as used herein, refer to the time course of administering a substance (e.g., an anti-IL-12, anti-IL-23 antibody) to a subject to achieve a therapeutic objective, wherein the time course is every other week (eow). The biweekly dosing regimen is not intended to include a weekly dosing regimen. Preferably, the substance is administered every 9-19 days, more preferably, every 11-17 days, even more preferably, every 13-15 days, and most preferably, every 14 days.

As used herein, the term “dose amount” refers to the quantity, e.g., milligrams (mg), of the substance which is administered to the subject. In one embodiment, the dose amount is a fixed dose, e.g., is not dependent on the weight of the subject to which the substance is administered. In another embodiment, the dose amount is not a fixed dose, e.g., is dependent on the weight of the subject to which the substance is administered. Exemplary dose amounts, e.g., fixed dose amounts, for use in the methods of the invention include, about 100 mg, about 110 mg, about 120 mg, about 130 mg, about 140 mg, about 150 mg, about 160 mg, about 170 mg, about 180 mg, or about 190 mg, about 200 mg, about 210 mg, about 220 mg, about 230 mg, about 240 mg, about 250 mg, about 260 mg, about 270 mg, about 280 mg, about 290 mg, or about 300 mg. In one embodiment, the dose amount is about 100 to about 300 mg. In yet another embodiment, the dose amount is about 100 to about 200 mg. Ranges intermediate to the above-recited ranges are also contemplated by the invention. For example, ranges having any one of these values as the upper or lower limits are also intended to be part of the invention, e.g., about 110 mg to about 170 mg, about 150 mg to about 220 mg, etc.

As used herein, the term “periodicity” as it relates to the administration of a substance (e.g., an antibody which binds to the p40 subunit of-IL-12 and/or -IL-23) refers to a (regular) recurring cycle of administering the substance to a subject. In one embodiment, the recurring cycle of administration of the substance to the subject achieves a therapeutic objective. The periodicity of administration of the substance may be about once a week, once every other week, about once every three weeks, about once every 4 weeks, about once every 5 weeks, about once every 6 weeks, about once every 7 weeks, about once every 8 weeks, about once every 9 weeks, about once every 10 weeks, about once every 11 weeks, about once every 12 weeks, about once every 13 weeks, about once every 14 weeks, about once every 15 weeks, about once every 16 weeks, about once every 17 weeks, about once every 18 weeks, about once every 19 to weeks, about once every 20 weeks, about once every 21 weeks, about once every 22 weeks, about once every 23 weeks, about once every 24 weeks, about once every 5-10 days, about once every 10-20 days, about once every 10-50 days, about once every 10-100 days, about once every 10-200 days, about once every 25-35 days, about once every 20-50 days, about once every 20-100 days, about once every 20-200 days, about once every 30-50 days, about once every 30-90 days, about once every 30-100 days, about once every 30-200 days, about once every 50-150 days, about once every 50-200 days, about once every 60-180 days, or about once every 80-100 days. Periodicities intermediate to the above-recited times are also contemplated by the invention. Ranges intermediate to the above-recited ranges are also contemplated by the invention. For example, ranges having any one of these values as the upper or lower limits are also intended to be part of the invention, e.g., about 110 days to about 170 days, or about 160 days to about 220 days.

As used herein, the phrase “periodicity of about once every 4 weeks” as it relates to the administration of a substance (e.g., an antibody which binds to the p40 subunit of-IL-12 and/or IL-23), refers to a (regular) recurring cycle of administering the substance to a subject about once every 4 weeks, about once every 28 days, or about once every month. In one embodiment, the recurring cycle of administration of the substance to the subject achieves or maintains a therapeutic objective (e.g., treating psoriasis), either alone or in conjunction with other recurring cycles (e.g., if a first periodicity, then in conjunction with a second and/or third periodicity; if a second periodicity, then in conjunction with a first and/or third periodicity; and if a third periodicity, then in conjunction with a first and second periodicity) of administering the substance. Preferably, the substance is administered once every 22-34 days, every 24-32 days, even more preferably, every 26-30 days (e.g., every 26, 27, 28, 29 or 30 days), and most preferably every 28 days.

As used herein, the phrase “periodicity of about once every 12 weeks” as it relates to the administration of a substance (e.g., an antibody which binds to the p40 subunit of-IL-12 and/or IL-23), refers to a (regular) recurring cycle of administering the substance to a subject about once every 12 weeks, about once every 84 days, or about once every 3 months. In one embodiment, the recurring cycle of administration of the substance to the subject achieves or maintains a therapeutic objective (e.g., treating psoriasis), either alone or in conjunction with other recurring cycles (e.g., if a first to periodicity, then in conjunction with a second and/or third periodicity; if a second periodicity, then in conjunction with a first and/or third periodicity; and if a third periodicity, then in conjunction with a first and second periodicity) of administering the substance. Preferably, the substance is administered once every 78-90 days, every 80-88 days, even more preferably, every 82-86 days (e.g., every 82, 83, 84, 85 or 86 days), and most preferably every 84 days.

The “duration of a periodicity” refers to a time over which the recurring cycle of administration occurs.

For example, a duration of the periodicity of administration of a substance may be may about 12 weeks during which the periodicity of administration is about once every week. For example, a duration of the periodicity may be about 6 weeks during which the periodicity of administration is about once every 4 weeks, e.g., the substance is administered at week zero and at week four.

The duration of periodicity may be about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 15 weeks, about 20 weeks, about 25 weeks, about 30 weeks, about 35 weeks, about 40 weeks, about 45 weeks, about 50 weeks, about 52 weeks, about 55 weeks, about 60 weeks, about 70 weeks, about 80 weeks, about 90 weeks, or about 100 weeks, or longer. In one embodiment, the duration of periodicity is for a length of time necessary or required to achieve a therapeutic objective, e.g., treatment, maintenance of treatment, etc. e.g., maintain a PASI 50, PASI 75, PASI 90, PASI 100 score or PGA of 0 or 1 score. Durations of a periodicity intermediate to the above-recited times are also contemplated by the invention.

The duration of periodicity may be about 4 weeks, about 8 weeks, about 12 weeks, about 16 weeks, about 20 weeks, about 24 weeks, about 28 weeks, about 32 weeks, about 36 weeks, about 40 weeks, about 44 weeks, about 48 weeks, about 52 weeks, or longer. The duration of periodicity may be at least about 4 weeks, at least about 8 weeks, at least about 12 weeks, at least about 16 weeks, at least about 20 weeks, at least about 24 weeks, at least about 28 weeks, at least about 32 weeks, at least about 36 weeks, at least about 40 weeks, at least about 44 weeks, at least about 48 weeks, or at least about 52 weeks.

Furthermore, the duration of periodicity may be at least about 1 week, at least to about 2 weeks, at least about 3 weeks, at least about 4 weeks, at least about 5 weeks, at least about 6 weeks, at least about 7 weeks, at least about 8 weeks, at least about 9 weeks, at least about 10 weeks, at least about 11 weeks, at least about 12 weeks, at least about 15 weeks, at least about 20 weeks, at least about 25 weeks, at least about 30 weeks, at least about 35 weeks, at least about 40 weeks, at least about 45 weeks, at least about 50 weeks, at least about 55 weeks, at least about 60 weeks, at least about 70 weeks, at least about 80 weeks, at least about 90 weeks, or at least about 100 weeks.

The term “combination” as in the phrase “a first agent in combination with a second agent” includes co-administration of a first agent and a second agent, which for example may be dissolved or intermixed in the same pharmaceutically acceptable carrier, or administration of a first agent, followed by the second agent, or administration of the second agent, followed by the first agent. The present invention, therefore, includes methods of combination therapeutic treatment and combination pharmaceutical compositions.

The term “concomitant” as in the phrase “concomitant therapeutic treatment” includes administering an agent in the presence of a second agent. A concomitant therapeutic treatment method includes methods in which the first, second, third, or additional agents are co-administered. A concomitant therapeutic treatment method also includes methods in which the first or additional agents are administered in the presence of a second or additional agents, wherein the second or additional agents, for example, may have been previously administered. A concomitant therapeutic treatment method may be executed step-wise by different actors. For example, one actor may administer to a subject a first agent and a second actor may to administer to the subject a second agent, and the administering steps may be executed at the same time, or nearly the same time, or at distant times, so long as the first agent (and additional agents) are after administration in the presence of the second agent (and additional agents). The actor and the subject may be the same entity (e.g., human).

The term “combination therapy”, as used herein, refers to the administration of two or more therapeutic substances, e.g., an anti-IL-12, anti-IL-23 antibody and another drug. The other drug(s) may be administered concomitant with, prior to, or following the administration of an anti-IL-12, anti-IL-23 antibody.

The term “kit” as used herein refers to a packaged product comprising components with which to administer the anti-IL-12, anti-IL-23 antibody of the to invention for treatment of a IL-12 related disorder. The kit preferably comprises a box or container that holds the components of the kit. The box or container is affixed with a label or a Food and Drug Administration approved protocol. The box or container holds components of the invention which are preferably contained within plastic, polyethylene, polypropylene, ethylene, or propylene vessels. The vessels can be capped-tubes or bottles. The kit can also include instructions for administering an anti-IL-12, anti-IL-23 antibody.

“At least one” is understood to be 1, 2, 3, 4, 5, 6, 7, 8, 9, 10, or more.

Various aspects of the invention are described in further detail in the following subsections.

I. Human Antibodies that Bind the p40 Subunit of Human IL-12 and/or Human IL-23

The present invention provides methods and compositions for using human antibodies, or antigen-binding portions thereof, that bind to an epitope of the p40 subunit of human IL-12 and/or IL-23 for the treatment of a disorder in which the activity of the p40 subunit of human IL-12 and/or IL-23 is detrimental, e.g., psoriasis. In addition to binding IL-12 and/or IL-23, the antibodies, or antigen binding portions thereof, further have at least one of the pharmacokinetic properties determined using a population pharmacokinetic model described herein. In certain embodiments, the antibodies also include one or more pharmacokinetic properties as determined using a single compartment model. In certain embodiments, the antibodies, or antigen binding portions thereof, have one or more activities, e.g., in a PHA assay or in an IFNγ production assay, or in a binding assay, e.g., specific K_{d} and/or K_{off}, as provided herein.

In one embodiment, the antibody used in the pharmaceutical composition of the invention is the antibody J695 (see U.S. Pat. No. 6,914,128, the entire contents of which are expressly incorporated by reference herein). J695 is a fully human antibody against interleukin 12 (IL-12) and IL-23. It binds with great affinity to the p40 subunit common to both IL-12 and IL-23, validated targets in the treatment of psoriasis (Ps).

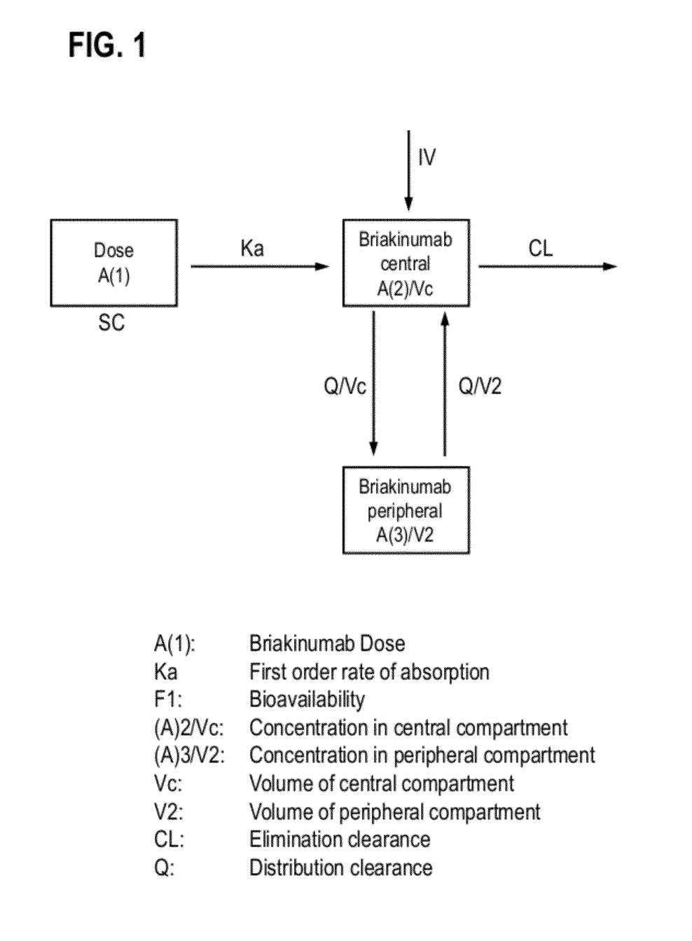

Antibodies that bind to the p40 subunit of human IL-12 and/or IL-23 can be selected, for example, by screening one or more human V_{L} and V_{H} cDNA libraries with hIL-12, such as by phage display techniques as described in Example 1 of U.S. Pat. No. 6,914,128. Screening of human V_{L} and V_{H} cDNA libraries initially identified a series of anti-IL-12 antibodies of which one antibody, referred to herein as “Joe 9” (or “Joe 9 wild type”), was selected for further development. Joe 9 is a relatively low affinity human IL-12 antibody (e.g., a K_{off} of about 0.1 sec⁻¹), yet is useful for specifically binding and detecting the p40 subunit of hIL-12. The affinity of the Joe 9 antibody was improved by conducting mutagenesis of the heavy and light chain CDRs, producing a panel of light and heavy chain variable regions that were “mixed and matched” and further mutated, leading to numerous additional anti-hIL-12 antibodies with increased affinity for the p40 subunit of hIL-12 (see Example 1, Table 2 of U.S. Pat. No. 6,914,128, incorporated herein by reference) and the sequence alignments of FIGS. 1A-D of U.S. Pat. No. 6,914,128 (which are expressly incorporated herein by reference).

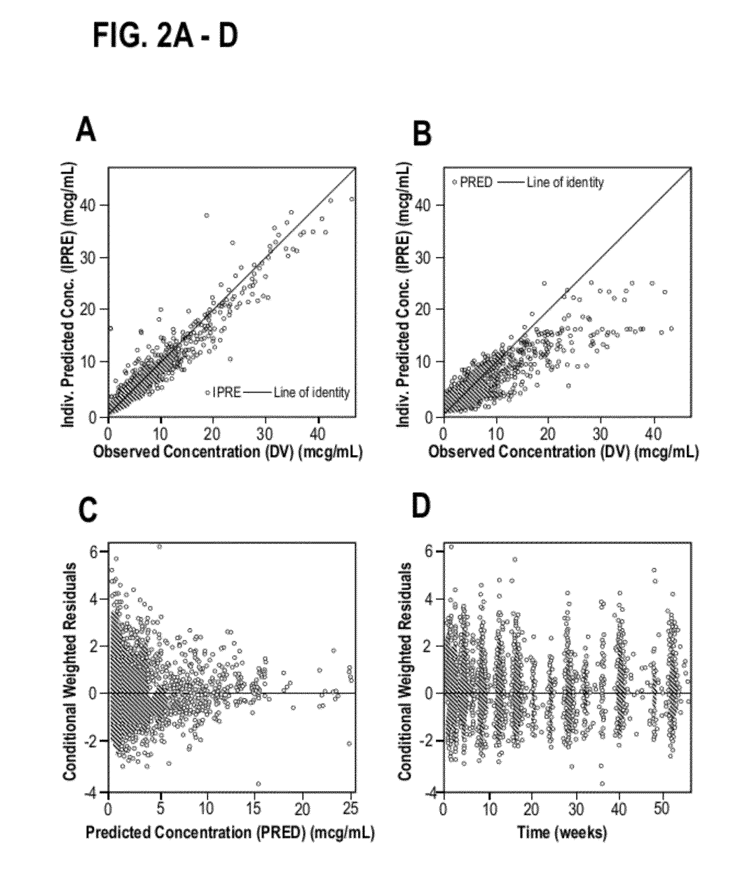

Of these antibodies, the human anti-hIL-12 antibody referred to herein as Y61 demonstrated a significant improvement in binding affinity (e.g., a K_{off} of about 2×10⁻⁴ sec⁻¹). The Y61 anti-hIL-12 antibody was selected for further affinity maturation by individually mutating specific amino acids residues within the heavy and light chain CDRs Amino acids residues of Y61 were selected for site-specific mutation (selective mutagenesis approach) based on the amino acid residue occupying a preferred selective mutagenesis position, contact and/or a hypermutation position. A summary of the substitutions at selected positions in the heavy and light chain CDRs is shown in FIGS. 2A-2H of U.S. Pat. No. 6,914,128 (which are expressly incorporated herein by reference). A preferred recombinant neutralizing antibody of the invention, referred to herein as J695, resulted from a Gly to Tyr substitution at position 50 of the light chain CDR2 of Y61, and a Gly to Tyr substitution at position 94 of the light chain CDR3 of Y61.

Amino acid sequence alignments of the heavy and light chain variable regions of a panel of anti-IL-12 antibodies used in the invention, on the lineage from Joe 9 wild type to J695, are shown in FIGS. 1A-1D of U.S. Pat. No. 6,914,128 (which are expressly incorporated herein by reference). These sequence alignments allowed for the identification of consensus sequences for preferred heavy and light chain variable regions of antibodies of the invention that bind the p40 subunit of hIL-12, as well as consensus sequences for the CDR3, CDR2, and CDR1, on the lineage from Joe 9 to J695. Moreover, the Y61 mutagenesis analysis summarized in FIGS. 2A-2H of U.S. Pat. No. 6,914,128 (which are expressly incorporated herein by reference) allowed for the to identification of consensus sequences for heavy and light chain variable regions that bind the p40 subunit of hIL-12, as well as consensus sequences for the CDR3, CDR2, and CDR1 that bind the p40 subunit of hIL-12 on the lineage from Y61 to J695 that encompasses sequences with modifications from Y61 yet that retain good hIL-12 binding characteristics. Preferred CDR, VH, and VL sequences of the invention (including consensus sequences) as identified by sequence identifiers in the attached Sequence Listing, are summarized in Table 1 below.

TABLE 1
Consensus Sequences for CDR and Heavy and
Light Chains of IL-12 Binding Antibodies

| SEQ | | | |
|---|---|---|---|
| ID | ANTIBODY | | |
| NO: | CHAIN | REGION | SEQUENCE |
| 1 | Consensus | CDR H3 | (H/S)-G-S-(H/Y)-D-(N/T/Y) |
| | Joe 9 to | | |
| | J695 | | |
| 2 | Consensus | CDR L3 | Q-(S/T)-Y-(D/E)-(S/R/K)- |
| | Joe 9 to | | (S/G/Y)-(L/F/T/S)- |
| | J695 | | (R/S/T/W/H)-(G/P)-(S/T/A/L)- |
| | | | (R/S/M/T/L)-(V/I/T/M/L) |
| 3 | Consensus | CDR H2 | F-I-R-Y-D-G-S-N-K-Y-Y-A-D-S- |
| | Joe 9 to | | V-K-G |
| | J695 | | |
| 4 | Consensus | CDR L2 | (G/Y)-N-(D/S)-(Q/N)-R-P-S |
| | Joe 9 to | | |
| | J695 | | |
| 5 | Consensus | CDR H1 | F-T-F-S-(S/E)-Y-G-M-H |
| | Joe 9 to | | |
| | J695 | | |
| 6 | Consensus | CDR L1 | (S/T)-G-(G/S)-(R/S)-S-N-I- |
| | Joe 9 to | | (G/V)-(S/A)-(N/G/Y)-(T/D)-V- |
| | J695 | | (K/H) |
| 7 | Consensus | VH | (full VH sequence; see |
| | Joe 9 to | | sequence listing) |
| | J695 | | |
| 8 | Consensus | VL | (full VL sequence; see |
| | Joe 9 to | | sequence listing) |
| | J695 | | |
| 9 | Consensus | CDR H3 | H-(G/V/C/H)-(S/T)- |
| | Y61 to | | (H/T/V/R/I)-(D/S)- |
| | J695 | | (N/K/A/T/S/F/W/H) |
| 10 | Consensus | CDR L3 | Q-S-Y-(D/S)-(Xaa)- |
| | Y61 to | | (G/D/Q/L/F/R/H/N/Y)-T-H-P-A- |
| | J695 | | L-L |
| 11 | Consensus | CDR H2 | (F/T/Y)-I-(R/A)-Y-(D/S/E/A)- |
| | Y61 to | | (G/R)-S-(Xaa)-K-(Y/E)-Y-A-D- |
| | J695 | | S-V-K-G |
| 12 | Consensus | CDR L2 | (G/Y/S/T/N/Q)-N-D-Q-R-P-S |
| | Y61 to | | |
| | J695 | | |
| 13 | Consensus | CDR H1 | F-T-F-(Xaa)-(Xaa)-(Y/H)- |
| | Y61 to | | (G/M/A/N/S)-M-H |
| | J695 | | |
| 14 | Consensus | CDR L1 | S-G-G-R-S-N-I-G- |
| | Y61 to | | (S/C/R/N/D/T)-(N/M/I)- |
| | J695 | | (T/Y/D/H/K/P)-V-K |
| 15 | Consensus | VH | (full VH sequence; see |
| | Y61 to | | sequence listing) |
| | J695 | | |
| 16 | Consensus | VL | (full VL sequence; see |
| | Y61 to | | sequence listing) |
| | J695 | | |
| 17 | Y61 | CDR H3 | H-G-S-H-D-N |
| 18 | Y61 | CDR L3 | Q-S-Y-D-R-G-T-H-P-A-L-L |
| 19 | Y61 | CDR H2 | F-I-R-Y-D-G-S-N-K-Y-Y-A-D-S- |
| | | | V-K-G |
| 20 | Y61 | CDR L2 | G-N-D-Q-R-P-S |
| 21 | Y61 | CDR H1 | F-T-F-S-S-Y-G-M-H |
| 22 | Y61 | CDR L1 | S-G-G-R-S-N-I-G-S-N-T-V-K |
| 23 | Y61 | VH | (full VH sequence; see |
| | | | sequence listing) |
| 24 | Y61 | VL | (full VL sequence; see |
| | | | sequence listing) |
| 25 | J695 | CDR H3 | H-G-S-H-D-N |
| 26 | J695 | CDR L3 | Q-S-Y-D-R-Y-T-H-P-A-L-L |
| 27 | J695 | CDR H2 | F-I-R-Y-D-G-S-N-K-Y-Y-A-D-S- |
| | | | V-K-G |
| 28 | J695 | CDR L2 | Y-N-D-Q-R-P-S |
| 29 | J695 | CDR H1 | F-T-F-S-S-Y-G-M-H |
| 30 | J695 | CDR L1 | S-G-S-R-S-N-I-G-S-N-T-V-K |
| 31 | J695 | VH | (full VH sequence; see |
| | | | sequence listing) |
| 32 | J695 | VL | (full VL sequence; see |
| | | | sequence listing) |

Antibodies produced from affinity maturation of Joe 9 wild type were functionally characterized by surface plasmon resonance analysis to determine the K_{d} and K_{off} rate. A series of antibodies were produced having a K_{off} rate within the range of about 0.1 s⁻¹ to about 1×10⁻⁵ s⁻¹, and more preferably a K_{off} of about 1×10⁻⁴ s⁻¹ to 1×10⁻⁵ s⁻¹ or less. Antibodies were also characterized in vitro for their ability to inhibit phytohemagglutinin (PHA) blast proliferation, as described in Example 3 of U.S. Pat. No. 6,914,128 (is the entire contents of which are expressly incorporated by reference herein). A series of antibodies were produced having an IC₅₀ value in the range of about 1×10⁻⁶ M to about 1×10⁻¹¹ M, more preferably about 1×10⁻¹⁰ M to 1×10⁻¹¹M or less.

Accordingly, in one aspect, the invention provides methods and compositions for using an isolated human antibody, or antigen-binding portion thereof, that binds to the p40 subunit of human IL-12 and/or IL-23 and dissociates from the p40 subunit of human IL-12 and/or IL-23 with a K_{off} rate constant of 0.1 s⁻¹ or less, as determined by surface plasmon resonance, or which inhibits phytohemagglutinin blast proliferation in an in vitro phytohemagglutinin blast proliferation assay (PHA assay) with an IC₅₀ of 1×10⁻⁶ M or less. In preferred embodiments, the isolated human p40 IL-12 and/or IL-23 antibody, or an antigen-binding portion thereof, dissociates from the p40 subunit of human IL-12 and/or IL-23 with a K_{off} rate constant of 1×10⁻² s⁻¹ or less, or inhibits phytohemagglutinin blast proliferation in an in vitro PHA assay with an IC₅₀ of 1×10⁻⁷ M or less. In more preferred embodiments, the isolated human p40 IL-12 and/or IL-23 antibody, or an antigen-binding portion thereof, dissociates from the p40 subunit of human IL-12 and/or IL-23 with a K_{off} rate constant of 1×10⁻³ s⁻¹ or less, or inhibits phytohemagglutinin blast proliferation in an in vitro PHA assay with an IC₅₀ of 1×10⁻⁸ M or less. In more preferred embodiments, the isolated human p40 IL-12 and/or IL-23 antibody, or an antigen-binding portion thereof, dissociates from the p40 subunit of human IL-12 and/or IL-23 with a K_{off} rate constant of 1×10⁻⁴ s⁻¹ or less, or inhibits phytohemagglutinin blast proliferation in an in vitro PHA assay with an IC₅₀ of 1×10⁻⁹ M or less. In more preferred embodiments, the isolated human p40 IL-12 and/or IL-23 antibody, or an antigen-binding portion thereof, dissociates from human IL-12 and/or IL-23 with a K_{off} rate constant of 1×10⁻⁵ s⁻¹ or less, or inhibits phytohemagglutinin blast proliferation in an in vitro PHA assay with an IC₅₀ of 1×10⁻¹⁰ M or less. In even more preferred embodiments, the isolated human p40 IL-12 and/or IL-23 antibody, or an antigen-binding portion thereof, dissociates from the p40 subunit of human IL-12 and/or to IL-23 with a K_{off} rate constant of 1×10⁻⁵ s⁻¹ or less, or inhibits phytohemagglutinin blast proliferation in an in vitro PHA assay with an IC₅₀ of 1×10⁻¹¹ M or less.

The dissociation rate constant (K_{off}) of a p40 subunit of IL-12 and/or IL-23 antibody can be determined by surface plasmon resonance (see Example 5 of U.S. Pat. No. 6,914,128, which is expressly incorporated by reference herein). Generally, surface plasmon resonance analysis measures real-time binding interactions between ligand (recombinant human IL-12 or p40 subunit of IL-12/IL-23 immobilized on a biosensor matrix) and analyte (antibodies in solution) by surface plasmon resonance (SPR) using the BIAcore system (Pharmacia Biosensor, Piscataway, N.J.). Surface plasmon analysis can also be performed by immobilizing the analyte (antibodies on a biosensor matrix) and presenting the ligand (recombinant IL-12, IL-23 or p40 in solution). Neutralization activity of p40 subunit of IL-12 and/or IL-23 antibodies, or antigen binding portions thereof, can be assessed using one or more of several suitable in vitro assays (see Example 3 of U.S. Pat. No. 6,914,128, incorporated herein by reference).

It is well known in the art that antibody heavy and light chain CDRs play an important role in the binding specificity/affinity of an antibody for an antigen. Accordingly, the invention encompasses human antibodies having light and heavy chain CDRs of Joe 9, as well as other antibodies having CDRs that have been modified to improve the binding specificity/affinity of the antibody. As demonstrated in Example 1 of U.S. Pat. No. 6,914,128, a series of modifications to the light and heavy chain CDRs results in affinity maturation of human anti-hIL-12 p40 antibodies. The heavy and light chain variable region amino acid sequence alignments of a series of human antibodies ranging from Joe 9 wild type to J695 that bind the p40 subunit of human IL-12 is shown in FIGS. 1A-1D of U.S. Pat. No. 6,914,128 (which are expressly incorporated herein by reference). Consensus sequence motifs for the CDRs of antibodies can be determined from the sequence alignment. For example, a consensus motif for the VH CDR3 of the lineage from Joe 9 to J695 comprises the amino acid sequence: (H/S)-G-S-(H/Y)-D-(N/T/Y) (SEQ ID NO: 1), which encompasses amino acids from position 95 to 102 of the consensus HCVR shown in SEQ ID NO: 7. A consensus motif for the VL CDR3 comprises the amino acid sequence: Q-(S/T)-Y-(D/E)-(S/R/K)-(S/G/Y)-(L/F/T/S)-(R/S/T/W/H)-(G/P)-(S/T/A/L)-(R/S/M/T/L-V/I/T/M/L) (SEQ ID NO: 2), which encompasses amino acids from position 89 to 97 of the consensus LCVR shown to in SEQ ID NO: 8.

Accordingly, in another aspect, the invention provides methods and compositions comprising an isolated human antibody, or an antigen-binding portion thereof, which has:

a) inhibits phytohemagglutinin blast proliferation in an in vitro PHA assay with an IC₅₀ of 1×10⁻⁶ M or less;

b) has a heavy chain CDR3 comprising the amino acid sequence of SEQ ID NO: 1; and

c) has a light chain CDR3 comprising the amino acid sequence of SEQ ID NO: 2; and

has one or more (e.g., 1, 2, 3, 4, 5, or 6) of the following properties as determined using a two compartment model:

d) a rate of clearance (C_{L}) of about 0.5 to about 1.0 L/day (e.g., about 0.64 to about 0.92 L/day; about 0.71 to about 0.85 L/day; about 0.779 L/day);

e) an absorption constant (kₐ) of about 0.4 to about 0.8 L/day (e.g., about 0.47 to about 0.75 L/day; about 0.54 to about 0.68 L/day; about 0.614 L/day);

f) volume of central compartment distribution (V_{c}) of about 3.5 to about 8.5 L (e.g., about 4.48 to about 7.60 L; about 5.26 to about 6.82 L; about 6.04 L);

g) second (peripheral compartment) volume (V₂) of about 2.2 to about 4.2 L (e.g., about 2.57 to about 3.79 L; about 2.88 to about 3.48 L; about 3.18 L);

h) rate of clearance from the first compartment to the second compartment (Q) of about 0.6 to about 1.1 L/day (e.g., about 0.6 to about 1.0 L/day; about 0.7 to about 0.9 L/day; about 0.805 L/day); and

i) bioavailability (F1) of about 0.29 to about 0.50 (e.g., about 0.32 to about 0.47; about 0.35 to about 0.43; about 0.392).

In a preferred embodiment, the antibody further comprises a VH CDR2 comprising the amino acid sequence: F-I-R-Y-D-G-S-N-K-Y-Y-A-D-S-V-K-G (SEQ ID NO: 3) (which encompasses amino acids from position 50 to 65 of the consensus HCVR comprising the amino acid sequence SEQ ID NO: 7) and further comprises a VL CDR2 comprising the amino acid sequence: (G/Y)-N-(D/S)-(Q/N)-R-P-S (SEQ ID NO: 4) (which encompasses amino acids from position 50 to 56 of the consensus LCVR comprising the amino acid sequence SEQ ID NO: 8).

In another preferred embodiment, the antibody further comprises a VH CDR1 comprising the amino acid sequence: F-T-F-S-(S/E)-Y-G-M-H (SEQ ID NO: 5) (which encompasses amino acids from position 27 to 35 of the consensus HCVR comprising the amino acid sequence SEQ ID NO: 7) and further comprises a VL CDR1 comprising the amino acid sequence: (S/T)-G-(G/S)-(R/S)-S-N-I-(G/V)-(S/A)-(N/G/Y)-(T/D)-V-(K/H) (SEQ ID NO: 6) (which encompasses amino acids from position 24 to 34 of the consensus LCVR comprising the amino acid sequence SEQ ID NO: 8).

In yet another preferred embodiment, the antibody used in the invention comprises a HCVR comprising the amino acid sequence of SEQ ID NO: 7 and a LCVR comprising the amino acid sequence of SEQ ID NO: 8.

Additional consensus motifs can be determined based on the mutational analysis performed on Y61 that led to the J695 antibody (summarized in FIGS. 2A-2H of U.S. Pat. No. 6,914,128, which are expressly incorporated herein by reference). As demonstrated by the graphs shown in FIGS. 2A-2H of U.S. Pat. No. 6,914,128 (which are expressly incorporated herein by reference), certain residues of the heavy and light chain CDRs of Y61 were amenable to substitution without significantly impairing the hIL-12 binding properties of the antibody. For example, individual substitutions at position 30 in CDR H1 with twelve different amino acid residues did not significantly reduce the K_{off} rate of the antibody, indicating that the position is amenable to substitution with a variety of different amino acid residues. Thus, based on the mutational analysis (i.e., positions within Y61 that were amenable to substitution by other amino acid residues) consensus motifs were determined. The consensus motifs for the heavy and light chain CDR3s are shown in SEQ ID NOs: 9 and 10, respectively, consensus motifs for the heavy and light chain CDR2s are shown in SEQ ID NOs: 11 and 12, respectively, and consensus motifs for the heavy and light chain CDR1s are shown in SEQ ID NOs: 13 and 14, respectively. Consensus motifs for the VH and VL regions are shown in SEQ ID NOs: 15 and 16, respectively.

Accordingly, in one aspect, the invention includes an isolated human antibody, or an antigen-binding portion thereof, which has one or more (e.g., 1, 2, 3, 4, 5, 6, 7, 8, or 9) of the following characteristics:

a) inhibits phytohemagglutinin blast proliferation in an in vitro PHA assay with an IC₅₀ of 1×10⁻⁹ M or less;

b) has a heavy chain CDR3 comprising the amino acid sequence of SEQ ID to NO: 9;

c) has a light chain CDR3 comprising the amino acid sequence of SEQ ID NO: 10; and

one or more (e.g., 1, 2, 3, 4, 5, or 6) of the following properties as determined using a two compartment model:

d) a rate of clearance (C_{L}) of about 0.5 to about 1.0 L/day (e.g., about 0.64 to about 0.92 L/day; about 0.71 to about 0.85 L/day; about 0.779 L/day);

e) an absorption constant (kₐ) of about 0.4 to about 0.8 L/day (e.g., about 0.47 to about 0.75 L/day; about 0.54 to about 0.68 L/day; about 0.614 L/day);

f) volume of central compartment distribution (V_{c}) of about 3.5 to about 8.5 L (e.g., about 4.48 to about 7.60 L; about 5.26 to about 6.82 L; about 6.04 L);

g) second (peripheral compartment) volume (V₂) of about 2.2 to about 4.2 L (e.g., about 2.57 to about 3.79 L; about 2.88 to about 3.48 L; about 3.18 L);

h) rate of clearance from the first compartment to the second compartment (Q) of about 0.6 to about 1.1 L/day (e.g., about 0.6 to about 1.0 L/day; about 0.7 to about 0.9 L/day; about 0.805 L/day); and

i) bioavailability (F1) of about 0.29 to about 0.50 (e.g., about 0.32 to about 0.47; about 0.35 to about 0.43; about 0.392).

In a preferred embodiment, the antibody further comprises a VH CDR2 comprising the amino acid sequence of SEQ ID NO: 11 and further comprises a VL CDR2 comprising the amino acid sequence of SEQ ID NO: 12.

In another preferred embodiment, the antibody further comprises a VH CDR1 comprising the amino acid sequence of SEQ ID NO: 13 and further comprises a VL CDR1 comprising the amino acid sequence of SEQ ID NO: 14.

In yet another preferred embodiment, the antibody used in the invention comprises a HCVR comprising the amino acid sequence of SEQ ID NO: 15 and a LCVR comprising the amino acid sequence of SEQ ID NO: 16.

A preferred antibody used in the invention, the human anti-hIL-12 antibody Y61, can be produced by affinity maturation of Joe 9 wild type by PCR mutagenesis of the CDR3 (as described in Example 1 of U.S. Pat. No. 6,914,128, which is expressly incorporated by reference herein). Y61 had an improved specificity/binding affinity determined by surface plasmon resonance and by in vitro neutralization assays. The heavy and light chain CDR3s of Y61 are shown in SEQ ID NOs: 17 and 18, to respectively, the heavy and light chain CDR2s of Y61 are shown in SEQ ID NOs: 19 and 20, respectively, and the heavy and light chain CDR1s of Y61 are shown in SEQ ID NOs: 21 and 22, respectively. The VH of Y61 has the amino acid sequence of SEQ ID NO: 23 and the VL of Y61 has the amino acid sequence of SEQ ID NO: 24 (these sequences are also shown in FIGS. 1A-1D of U.S. Pat. No. 6,914,128 (which are expressly incorporated herein by reference) aligned with Joe9).

Accordingly, in another aspect, the invention features use of an isolated human antibody, or an antigen-binding portion thereof, which has at least one (e.g., 1, 2, 3, 4, 5, 6, 7, 8, or 9) of the following characteristics:

a) inhibits phytohemagglutinin blast proliferation in an in vitro PHA assay with an IC₅₀ of 1×10⁻⁹ M or less;

b) has a heavy chain CDR3 comprising the amino acid sequence of SEQ ID NO: 17;

c) has a light chain CDR3 comprising the amino acid sequence of SEQ ID NO: 18; and

one or more (e.g., 1, 2, 3, 4, 5, or 6) of the following properties as determined using a two compartment model:

d) a rate of clearance (C_{L}) of about 0.5 to about 1.0 L/day (e.g., about 0.64 to about 0.92 L/day; about 0.71 to about 0.85 L/day; about 0.779 L/day);

e) an absorption constant (kₐ) of about 0.4 to about 0.8 L/day (e.g., about 0.47 to about 0.75 L/day; about 0.54 to about 0.68 L/day; about 0.614 L/day);

f) volume of central compartment distribution (V_{c}) of about 3.5 to about 8.5 L (e.g., about 4.48 to about 7.60 L; about 5.26 to about 6.82 L; about 6.04 L);

g) second (peripheral compartment) volume (V₂) of about 2.2 to about 4.2 L (e.g., about 2.57 to about 3.79 L; about 2.88 to about 3.48 L; about 3.18 L);

h) rate of clearance from the first compartment to the second compartment (Q) of about 0.6 to about 1.1 L/day (e.g., about 0.6 to about 1.0 L/day; about 0.7 to about 0.9 L/day; about 0.805 L/day); and

i) bioavailability (F1) of about 0.29 to about 0.50 (e.g., about 0.32 to about 0.47; about 0.35 to about 0.43; about 0.392).

In a preferred embodiment, the isolated human antibody, or an antigen-binding portion thereof, used in the methods and compositions of the invention has a heavy chain CDR2 comprising the amino acid sequence of SEQ ID NO: 19 and a light chain CDR2 comprising the amino acid sequence of SEQ ID NO: 20.

In another preferred embodiment, the isolated human antibody, or an antigen-binding portion thereof, used in the methods and compositions of the invention, has a heavy chain CDR1 comprising the amino acid sequence of SEQ ID NO: 21 and a light chain CDR1 comprising the amino acid sequence of SEQ ID NO: 22.

In yet another preferred embodiment, the isolated human antibody, or an antigen-binding portion thereof, used in the methods and compositions of the invention comprising a the heavy chain variable region comprising the amino acid sequence of SEQ ID NO: 23, and a light chain variable region comprising the amino acid sequence of SEQ ID NO: 24.

In certain embodiments, the full length antibody comprises a heavy chain constant region, such as IgG1, IgG2, IgG3, IgG4, IgM, IgA and IgE constant regions, and any allotypic variant therein as described in Kabat (Kabat, E. A., et al. (1991) Sequences of Proteins of Immunological Interest, Fifth Edition, U.S. Department of Health and Human Services, NIH Publication No. 91-3242). Preferably, the antibody heavy chain constant region is an IgG1 heavy chain constant region. Alternatively, the antibody portion can be an Fab fragment, an F(ab′₂) fragment or a single chain Fv fragment.

Modifications of individual residues of Y61 led to the production of a panel of antibodies shown in FIGS. 2A-2H of U.S. Pat. No. 6,914,128 (which are expressly incorporated herein by reference). The specificity/binding affinity of each antibody was determined by surface plasmon resonance and/or by in vitro neutralization assays.

In yet another preferred embodiment, the isolated human antibody, or an antigen-binding portion thereof, comprising a the heavy chain variable region comprising the amino acid sequence of SEQ ID NO: 23, and a light chain variable region comprising the amino acid sequence of SEQ ID NO: 24.

In certain embodiments, the full length antibody comprising a heavy chain constant region such as IgG1, IgG2, IgG3, IgG4, IgM, IgA and IgE constant regions and any allotypic variant therein as described in Kabat (, Kabat, E. A., et al. (1991) Sequences of Proteins of Immunological Interest, Fifth Edition, U.S. Department of Health and Human Services, NIH Publication No. 91-3242). Preferably, the antibody to heavy chain constant region is an IgG1 heavy chain constant region. Alternatively, the antibody portion can be a Fab fragment, an F(ab'₂) fragment or a single chain Fv fragment.

A particularly preferred recombinant, neutralizing antibody, J695, which may be used in the pharmaceutical compositions and methods of the invention was produced by site-directed mutagenesis of contact and hypermutation amino acids residues of antibody Y61 (see Example 2 of U.S. Pat. No. 6,914,128, which is expressly incorporated by reference herein). J695 differs from Y61 by a Gly to Tyr substitution in Y61 at position 50 of the light chain CDR2 and by a Gly to Tyr substitution at position 94 of the light chain CDR3. The heavy and light chain CDR3s of J695 are shown in SEQ ID NOs: 25 and 26, respectively, the heavy and light chain CDR2s of J695 are shown in SEQ ID NOs: 27 and 28, respectively, and the heavy and light chain CDR1s of J695 are shown in SEQ ID NOs: 29 and 30, respectively. The VH of J695 has the amino acid sequence of SEQ ID NO: 31 and the VL of J695 has the amino acid sequence of SEQ ID NO: 32 (these sequences are also shown in FIGS. 1A-1D of U.S. Pat. No. 6,914,128 (incorporated herein by reference), aligned with Joe9); and one or more of the pharmacokinetic properties provided herein as determined using a two compartment model.

Accordingly, in another aspect, the invention features an isolated human antibody, or an antigen-binding portion thereof, which a) inhibits phytohemagglutinin blast proliferation in an in vitro PHA assay with an IC₅₀ of 1×10⁻⁹ M or less; b) has a heavy chain CDR3 comprising the amino acid sequence of SEQ ID NO: 25; and c) has a light chain CDR3 comprising the amino acid sequence of SEQ ID NO: 26.

In preferred embodiment, the isolated human antibody, or an antigen-binding portion thereof, used in the invention has a heavy chain CDR2 comprising the amino acid sequence of SEQ ID NO: 27, and a light chain CDR2 comprising the amino acid sequence of SEQ ID NO: 28.

In another preferred embodiment, the isolated human antibody, or an antigen-binding portion thereof, used in the invention has a heavy chain CDR1 comprising the amino acid sequence of SEQ ID NO: 29, and a light chain CDR1 comprising the amino acid sequence of SEQ ID NO: 30.

In yet another preferred embodiment, the isolated human antibody, or an antigen-binding portion thereof, used in the invention has a heavy chain variable region comprising the amino acid sequence of SEQ ID NO: 31, and a light chain variable region comprising the amino acid sequence of SEQ ID NO: 32.

In certain embodiments, the full length antibody comprises a heavy chain constant region, such as IgG1, IgG2, IgG3, IgG4, IgM, IgA and IgE constant regions and any allotypic variant therein as described in Kabat (Kabat, E. A., et al. (1991) Sequences of Proteins of Immunological Interest, Fifth Edition, U.S. Department of Health and Human Services, NIH Publication No. 91-3242). Preferably, the antibody heavy chain constant region is an IgG1 heavy chain constant region. Alternatively, the antibody portion can be an Fab fragment, an F(ab'₂) fragment or a single chain Fv fragment.

Additional mutations in the preferred consensus sequences for CDR3, CDR2, and CDR1 of antibodies on the lineage from Joe 9 to J695, or from the lineage Y61 to J695, can be made to provide additional anti-IL-12 antibodies of the invention. Such methods of modification can be performed using standard molecular biology techniques, such as by PCR mutagenesis, targeting individual contact or hypermutation amino acid residues in the light chain and/or heavy chain CDRs, followed by kinetic and functional analysis of the modified antibodies as described herein (e.g., neutralization assays described in Example 3 of U.S. Pat. No. 6,914,128, and by BIAcore analysis, as described in Example 5 of U.S. Pat. No. 6,914,128, the entire contents of which is expressly incorporated by reference herein).

Accordingly, in another aspect the invention features pharmaceutical compositions and methods which use an isolated human antibody, or an antigen-binding portion thereof, which

a) inhibits phytohemagglutinin blast proliferation in an in vitro PHA assay with an IC₅₀ of 1×10⁻⁶ M or less;

b) comprises a heavy chain CDR3 comprising the amino acid sequence of SEQ ID NO: 1, a heavy chain CDR2 comprising the amino acid sequence of SEQ ID NO: 3 and a heavy chain CDR1 comprising the amino acid sequence of SEQ ID NO: 5, or a mutant thereof having one or more amino acid substitutions at a preferred selective mutagenesis position or a hypermutation position, wherein said mutant has a k_{off} rate no more than 10-fold higher than the antibody comprising a heavy chain CDR3 comprising the amino acid sequence of SEQ ID NO: 1, a heavy chain CDR2 comprising the amino to acid sequence of SEQ ID NO: 3, and a heavy chain CDR1 comprising the amino acid sequence of SEQ ID NO: 5;

c) comprises a light chain CDR3 comprising the amino acid sequence of SEQ ID NO: 2, a light chain CDR2 comprising the amino acid sequence of SEQ ID NO: 4, and a light chain CDR1 comprising the amino acid sequence of SEQ ID NO: 6, or a mutant thereof having one or more amino acid substitutions at a preferred selective mutagenesis position or a hypermutation position, wherein said mutant has a k_{off} rate no more than 10-fold higher than the antibody comprising a light chain CDR3 comprising the amino acid sequence of SEQ ID NO: 2, a light chain CDR2 comprising the amino acid sequence of SEQ ID NO: 4, and a light chain CDR1 comprising the amino acid sequence of SEQ ID NO: 6; and

d) one or more of the pharmacokinetic properties provided herein as determined using a two compartment model.

In another aspect the invention features use of an isolated human antibody, or an antigen-binding portion thereof, which

a) inhibits phytohemagglutinin blast proliferation in an in vitro PHA assay with an IC₅₀ of 1×10⁻⁹ M or less;

b) comprises a heavy chain CDR3 comprising the amino acid sequence of SEQ ID NO: 9, a heavy chain CDR2 comprising the amino acid sequence of SEQ ID NO: 11 and a heavy chain CDR1 comprising the amino acid sequence of SEQ ID NO: 13, or a mutant thereof having one or more amino acid substitutions at a preferred selective mutagenesis position, contact position or a hypermutation position, wherein said mutant has a k_{off} rate no more than 10-fold higher than the antibody comprising a heavy chain CDR3 comprising the amino acid sequence of SEQ ID NO: 9, a heavy chain CDR2 comprising the amino acid sequence of SEQ ID NO: 11, and a heavy chain CDR1 comprising the amino acid sequence of SEQ ID NO: 13; and

c) comprises a light chain CDR3 comprising the amino acid sequence of SEQ ID NO: 10, a light chain CDR2 comprising the amino acid sequence of SEQ ID NO: 12, and a light chain CDR1 comprising the amino acid sequence of SEQ ID NO: 14, or a mutant thereof having one or more amino acid substitutions at a preferred selective mutagenesis position, contact position or a hypermutation position, wherein said mutant has a k_{off} rate no more than 10-fold higher than the antibody comprising a light chain CDR3 comprising the amino acid sequence of SEQ ID NO: 10, a light chain to CDR2 comprising the amino acid sequence of SEQ ID NO: 12, and a light chain CDR1 comprising the amino acid sequence of SEQ ID NO: 14; and

d) one or more of the pharmacokinetic properties provided herein as determined using a two compartment model.

An ordinarily skilled artisan will also appreciate that additional mutations to the CDR regions of an antibody, for example in Y61 or in J695, can be made to provide additional anti-IL-12 antibodies for use in the pharmaceutical compositions and methods of the invention. Such methods of modification can be performed using standard molecular biology techniques, as described above. The functional and kinetic analysis of the modified antibodies can be performed as described in Example 3 of U.S. Pat. No. 6,914,128 and Example 5 of U.S. Pat. No. 6,914,128, respectively. Modifications of individual residues of Y61 that led to the identification of J695 are shown in FIGS. 2A-2H of U.S. Pat. No. 6,914,128 (which are incorporated herein by reference) and are described in Example 2 of U.S. Pat. No. 6,914,128 (which is expressly incorporated by reference herein).

Accordingly, in another aspect the invention features pharmaceutical compositions and methods which use an isolated human antibody, or an antigen-binding portion thereof, which

a) inhibits phytohemagglutinin blast proliferation in an in vitro PHA assay with an IC₅₀ of 1×10⁻⁹ M or less;

b) comprises a heavy chain CDR3 comprising the amino acid sequence of SEQ ID NO: 17, a heavy chain CDR2 comprising the amino acid sequence of SEQ ID NO: 19 and a heavy chain CDR1 comprising the amino acid sequence of SEQ ID NO: 21, or a mutant thereof having one or more amino acid substitutions at a preferred selective mutagenesis position or a hypermutation position, wherein said mutant has a k_{off} rate no more than 10-fold higher than the antibody comprising a heavy chain CDR3 comprising the amino acid sequence of SEQ ID NO: 17, a heavy chain CDR2 comprising the amino acid sequence of SEQ ID NO: 19, and a heavy chain CDR1 comprising the amino acid sequence of SEQ ID NO: 21; and

c) comprises a light chain CDR3 comprising the amino acid sequence of SEQ ID NO: 18, a light chain CDR2 comprising the amino acid sequence of SEQ ID NO: 20, and a light chain CDR1 comprising the amino acid sequence of SEQ ID NO: 22, or a mutant thereof having one or more amino acid substitutions at a preferred to selective mutagenesis position or a hypermutation position, wherein said mutant has a k_{off} rate no more than 10-fold higher than the antibody comprising a light chain CDR3 comprising the amino acid sequence of SEQ ID NO: 18, a light chain CDR2 comprising the amino acid sequence of SEQ ID NO: 20, and a light chain CDR1 comprising the amino acid sequence of SEQ ID NO: 22; and

d) one or more of the pharmacokinetic properties provided herein as determined using a two compartment model.

In another aspect the invention features use of an isolated human antibody, or an antigen-binding portion thereof, which

a) inhibits phytohemagglutinin blast proliferation in an in vitro PHA assay with an IC₅₀ of 1×10⁻⁹ M or less;

b) comprises a heavy chain CDR3 comprising the amino acid sequence of SEQ ID NO: 25, a heavy chain CDR2 comprising the amino acid sequence of SEQ ID NO: 27 and a heavy chain CDR1 comprising the amino acid sequence of SEQ ID NO: 29, or a mutant thereof having one or more amino acid substitutions at a preferred selective mutagenesis position or a hypermutation position, wherein said mutant has a k_{off} rate no more than 10-fold higher than the antibody comprising a heavy chain CDR3 comprising the amino acid sequence of SEQ ID NO: 25, a heavy chain CDR2 comprising the amino acid sequence of SEQ ID NO: 27, and a heavy chain CDR1 comprising the amino acid sequence of SEQ ID NO: 29; and

c) comprises a light chain CDR3 comprising the amino acid sequence of SEQ ID NO: 26, a light chain CDR2 comprising the amino acid sequence of SEQ ID NO: 28, and a light chain CDR1 comprising the amino acid sequence of SEQ ID NO: 30, or a mutant thereof having one or more amino acid substitutions at a preferred selective mutagenesis position or a hypermutation position, wherein said mutant has a k_{off} rate no more than 10-fold higher than the antibody comprising a light chain CDR3 comprising the amino acid sequence of SEQ ID NO: 26, a light chain CDR2 comprising the amino acid sequence of SEQ ID NO: 28, and a light chain CDR1 comprising the amino acid sequence of SEQ ID NO: 30; and

d) one or more of the pharmacokinetic properties provided herein as determined using a two compartment model.

In yet another embodiment, the invention provides use of an isolated human antibodies, or antigen-binding portions thereof, that neutralize the activity of the p40 to subunit of human IL-12, and at least one additional primate IL-12 selected from the group consisting of baboon IL-12, marmoset IL-12, chimpanzee IL-12, cynomolgus IL-12 and rhesus IL-12, but which do not neutralize the activity of the mouse p40 subunit of IL-12.

### II. Pharmaceutical Compositions and Pharmaceutical Administration

The antibodies and antibody-portions of the invention can be incorporated into pharmaceutical compositions suitable for administration to a subject. Typically, the pharmaceutical composition comprises an antibody or antibody portion of the invention and a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the antibody or antibody portion.

The antibodies and antibody-portions of the invention can be incorporated into a pharmaceutical composition suitable for parenteral administration. Preferably, the antibody or antibody-portions will be prepared as an injectable solution containing 0.1-250 mg/ml antibody. The injectable solution can be composed of either a liquid or lyophilized dosage form in a flint or amber vial, ampule or pre-filled syringe. The buffer can be L-histidine (1-50 mM), optimally 5-10 mM, at pH 5.0 to 7.0 (optimally pH 6.0). Other suitable buffers include but are not limited to, sodium succinate, sodium citrate, sodium phosphate or potassium phosphate. Sodium chloride can be used to modify the toxicity of the solution at a concentration of 0-300 mM (optimally 150 mM for a liquid dosage form). Cryoprotectants can be included for a lyophilized dosage form, principally 0-10% sucrose (optimally 0.5-1.0%). Other suitable cryoprotectants include trehalose and lactose. Bulking agents can be included for a lyophilized dosage form, principally 1-10% mannitol (optimally 2-4%). Stabilizers can be used in both to liquid and lyophilized dosage forms, principally 1-50 mM L-Methionine (optimally 5-10 mM). Other suitable bulking agents include glycine, arginine, can be included as 0-0.05% polysorbate-80 (optimally 0.005-0.01%). Additional surfactants include but are not limited to polysorbate 20 and BRIJ surfactants.

In one embodiment, the invention provides a formulation comprising the antibody in combination with a polyol, a surfactant, a stabilizer, and a buffer system with a pH of about 5 to 5. In one embodiment said formulation is free of metal. In a preferred embodiment, the formulation comprises the antibody and mannitol, histidine, methionine, polysorbate 80, hydrochloric acid, and water.

In one embodiment, an aqueous formulation is prepared comprising the antibody in a pH-buffered solution. The buffer of this invention has a pH ranging from about 4 to about 8, preferably from about 4.5 to about 7.5, more preferably from about 5 to about 7, more preferably from about 5.5 to about 6.5, and most preferably has a pH of about 6.0 to about 6.2. In a particularly preferred embodiment, the buffer has a pH of about 6. Ranges intermediate to the above recited pH's are also intended to be part of this invention. For example, ranges of values using a combination of any of the above recited values as upper and/or lower limits are intended to be included. Examples of buffers that will control the pH within this range include acetate (e.g. sodium acetate), succinate (such as sodium succinate), gluconate, histidine, citrate, phosphate and other organic acid buffers. In a preferred embodiment of the invention, the formulation contains a buffer system comprising histidine. In a preferred embodiment of the invention, the buffer is histidine, e.g., L-histidine. In preferred embodiments, the formulation of the invention comprises a buffer system comprising about 1-100 mM histidine, preferably about 5-50 mM histidine, and most preferably 10 mM histidine. One of skill in the art will recognize that sodium chloride can be used to modify the toxicity of the solution, e.g., at a concentration of 1-300 mM, and optimally 150 mM for a liquid dosage form.

A polyol, which acts as a tonicifier and may stabilize the antibody, is also included in the formulation. The polyol is added to the formulation in an amount that may vary with respect to the desired isotonicity of the formulation. Preferably the aqueous formulation is isotonic. The amount of polyol added may also vary with respect to the molecular weight of the polyol. For example, a lower amount of a monosaccharide (e.g., mannitol) may be added, compared to a disaccharide (such as to trehalose). In a preferred embodiment of the invention, the polyol that is used in the formulation as a tonicity agent is mannitol. In a preferred embodiment, the composition comprises about 10 to about 100 mg/ml, or about 20 to about 80, about 20 to about 70, about 30 to about 60, about 30 to about 50 mg/ml of mannitol, for example, about 10, about 20, about 30, about 40, about 50, about 60, about 70, about 80, about 90, and about 100 mg/ml of mannitol In a preferred embodiment, the formulation comprises about 40 mg/ml of mannitol (corresponding to about 4% mannitol). In a preferred embodiment, the composition comprises between about 1% to about 10% mannitol, more preferably between about 2% to about 6% mannitol, and most preferably about 4% mannitol. In another embodiment of the invention, the polyol sorbitol is included in the formulation.

A stabilizer or antioxidant is also added to the antibody formulation. A stabilizer can be used in both liquid and lyophilized dosage forms. Formulations of the invention preferably comprise the stabilizer methionine, e.g., L-Methionine. Other stabilizers useful in formulations of the invention are known to those of skill in the art and include, but are not limited to, glycine and arginine. Cryoprotectants can be included for a lyophilized dosage form, principally sucrose (e.g., 1-10% sucrose, and optimally 0.5-1.0% sucrose). Other suitable cyroprotectants include trehalose and lactose.

A detergent or surfactant is also added to the antibody formulation. Exemplary detergents include nonionic detergents such as polysorbates (e.g., polysorbates 20, 80 etc.) or poloxamers (e.g., poloxamer 188). The amount of detergent added is such that it reduces aggregation of the formulated antibody and/or minimizes the formation of particulates in the formulation and/or reduces adsorption. In a preferred embodiment of the invention, the formulation includes a surfactant that is a polysorbate. In another preferred embodiment of the invention, the formulation contains the detergent polysorbate 80 or Tween 80. Tween 80 is a term used to describe polyoxyethylene (20) sorbitanmonooleate (see Fiedler, Lexikon der Hifsstoffe, Editio Cantor Verlag Aulendorf, 4th ed., 1996). In one preferred embodiment, the formulation contains between 0.001 to about 0.1% polysorbate 80, or between about 0.005 and 0.05% polysorbate 80, for example, about 0.001, about 0.005, about 0.01, about 0.05, or about 0.1% polysorbate 80. In a preferred embodiment, about 0.01% polysorbate 80 is found in the formulation of the invention.

In a preferred embodiment of the invention, the formulation is a 1.0 mL solution to in a container containing the ingredients shown below in Table 1. In another embodiment, the formulation is a 0.8 mL solution in a container.

TABLE 1
A 1.0 mL Solution¹⁾ of J695 Formulation for Injection

| Name of Ingredient | Quantity | Function |
|---|---|---|
| Active substance: | | |
| Antibody (J695)²⁾ | 50.0 or | Active substance |
| | 100.0 mg | |
| Excipients: | | |
| Mannitol | 40 mg | Tonicity agent |
| Polysorbate 80 | 0.10 mg | Detergent/Surfactant |
| Histidine | 1.55 mg | Buffer |
| Methionine | 1.49 mg | Stabilizer |
| Water for injection | To 1 ml | Solvent |
| Hydrochloric Acid | q.s. | pH adjustment to 6.0 |

¹⁾Density of the solution: 1.0398 g/mL
²⁾Is used as concentrate

In one embodiment, the formulation is a formulation described in U.S. application Ser. No. 12/625,057, which published as U.S. 2010/0172862 A1, the entire contents of which are hereby expressly incorporated herein by reference.

In one embodiment, the formulation contains the above-identified agents (i.e., antibody, polyol, surfactant, stabilizer and buffer) and is essentially free of one or more preservatives, such as benzyl alcohol, phenol, m-cresol, chlorobutanol and benzethonium Cl. In another embodiment, a preservative may be included in the formulation, particularly where the formulation is a multidose formulation. One or more other pharmaceutically acceptable carriers, excipients or stabilizers such as those described in Remington's Pharmaceutical Sciences 16th edition, Osol, A. Ed. (1980) may be included in the formulation provided that they do not significantly adversely affect the desired characteristics of the formulation. Acceptable carriers, excipients or stabilizers are nontoxic to recipients at the dosages and concentrations employed and include; additional buffering agents; co-solvents; antioxidants such as ascorbic acid; to chelating agents such as EDTA; metal complexes (e.g. Zn-protein complexes); biodegradable polymers such as polyesters; and/or salt-forming counterions such as sodium.

In one embodiment, the formulations of the invention have improved properties as compared to art-recognized formulations. For example, the formulations of the invention have an improved shelf life and/or stability as compared to art recognized formulations. In one embodiment, the formulations of the invention have a shelf life of at least 18 months, e.g., in a liquid state or in a solid state. In another embodiment, the formulations of the invention have a shelf life of at least 24 months, e.g., in a liquid state or in a solid state. In a preferred embodiment, the formulations of the invention have a shelf life of at least 24 months at a temperature of 2-8° C. In a preferred embodiment, the formulations of the invention have a shelf life of at least 18 months or of at least 24 months at a temperature of between about −20 and −80° C. In another embodiment, the formulations of the invention maintain stability following at least 5 freeze/thaw cycles of the formulation. In a preferred aspect, the formulations of the invention comprise, e.g., an antibody, comprising at least a portion of a lambda light chain, e.g., J695, wherein the formulation provides enhanced resistance to fragmentation of the lambda light chain, e.g., reduced cleavage of the lambda light chain, as compared to art recognized formulations.

In one embodiment, the formulations of the invention are substantially free of metal. In one embodiment, the formulations of the invention are substantially free of a metal selected from the group consisting of Fe2+, Fe3+, Ca2+ and Cu1+. In one embodiment, the formulations of the invention comprise an amount of metal that is sufficiently low to reduce or prevent cleavage of the lambda chain in the presence of histidine, e.g., the metal is present at a concentration of less than about 5,060 ppb, less than about 1,060 ppb, less than about 560 ppb, less than about 500 ppb, less than about 450 ppb, less than about 400 ppb, less than about 350 ppb, less than about 310 ppb, less than about 300 ppb, less than about 250 ppb, less than about 200 ppb, less than about 160 ppb, less than about 150 ppb, less than about 140 ppb, less than about 130 ppb, less than about 120 ppb, less than about 110 ppb, less than about 100 ppb, less than about 90 ppb, less than about 80 ppb, less than about 70 ppb, less than about 60 ppb, less than about 50 ppb, less than about 40 ppb, less than about 30 ppb, less than about 20 ppb, less than about 10 ppb, or less than about 1 ppb. In one embodiment, the metal is present at to a concentration of less than about 160 ppb. In one embodiment, the metal is present at a concentration of less than about 110 ppb. In one embodiment, the metal is present at a concentration of less than about 70 ppb, e.g., a concentration of about 60 ppb. Maximum concentrations intermediate to the above recited concentrations, e.g., less than about 65 ppb, are also intended to be part of this invention. Further, ranges of values using a combination of any of the above recited values as upper and/or lower limits, e.g., concentrations between about 50 ppb and about 70 ppb, are also intended to be included.

In one embodiment, the formulations of the invention are substantially free of metal following subjection to at least one procedure that removes metal, such as filtration, buffer exchange, chromatography or resin exchange. Procedures useful to remove metal from formulations of the invention are known to one of skill in the art and are further described herein. In one embodiment, the formulations of the invention comprise a metal chelator, e.g., such that the molecule is not cleaved within the hinge region or is cleaved within the hinge region at a level which is less than the level of cleavage observed in the absence of the metal chelator. In the formulations of the invention, the metal chelator may be, for example, a siderophore, calixerenes, an aminopolycarboxylic acid, a hydroxyaminocarboxylic acid, an N-substituted glycine, a 2-(2-amino-2-oxoethyl)aminoethane sulfonic acid (BES), a bidentate, tridentate or hexadentate iron chelator, a copper chelator, and derivatives, analogues, and combinations thereof. Metal chelators useful in formulations of the invention are known to one of skill in the art, and are further described below.

Particular siderophores useful in formulations of the invention include, but are not limited to, aerobactin, agrobactin, azotobactin, bacillibactin, N-(5-C3-L (5 aminopentyl)hydroxycarbamoyl)-propionamido)pentyl)-3(5-(N-hydroxyacetoamido)-pentyl)carbamoyl)-proprionhydroxamic acid (deferoxamine, desferrioxamine or DFO or DEF), desferrithiocin, enterobactin, erythrobactin, ferrichrome, ferrioxamine B, ferrioxamine E, fluviabactin, fusarinine C, mycobactin, parabactin, pseudobactin, vibriobactin, vulnibactin, yersiniabactin, ornibactin, and derivatives, analogues, and combinations thereof.

Aminopolycarboxylic acids useful in formulations of the invention include, but are not limited to, ethylenediaminetetraacetic acid (EDTA), nitriloacetic acid (NTA), trans-diaminocyclohexane tetraacetic acid (DCTA), diethylenetriamine pentaacetic acid (DTPA), N-2-acetamido-2-iminodiacetic acid (ADA), aspartic acid, bis(aminoethyl)glycolether N,N,N′N′-tetraacetic acid (EGTA), glutamic acid, and N,N′-bis(2-hydroxybenzyl)ethylenediamine-N,N′-diacetic acid (HBED), and derivatives, analogues, and combinations thereof.

Hydroxyaminocarboxylic acids useful in formulations of the invention include, but are not limited to, N-hydroxyethyliminodiacetic acid (HIMDA), N,N-bishydroxyethylglycine (bicine), and N-(trishydroxymethylmethyl) glycine (tricine), and derivatives, analogues, and combinations thereof. N-substituted glycines, e.g., glycylglycine, as well as derivatives, analogues, or combinations thereof, are also useful as metal chelators in formulations of the invention. The metal chelator 2-(2-amino-2-oxoethyl)aminoethane sulfonic acid (BES), and derivatives, analogues, and combinations thereof, can also be used.

Particular calixarenes useful in formulations of the invention include, but are not limited to, a macrocycle or cyclic oligomer based on a hydroxyalkylation product of a phenol and an aldehyde, and derivatives, analogues, and combinations thereof. Particular copper chelators useful in the invention include triethylenetetramine (trientine), etraethylenepentamine, D-penicillamine, ethylenediamine, bispyridine, phenantroline, bathophenanthroline, neocuproine, bathocuproine sulphonate, cuprizone, cis,cis-1,3,5,-triaminocyclohexane (TACH), tachpyr, and derivatives, analogues, and combinations thereof.

Additional metal chelators that can be employed in formulations of the invention include a hydroxypyridine-derivate, a hydrazone-derivate, and hydroxyphenyl-derivate, or a nicotinyl-derivate, such as 1,2-dimethyl-3-hydroxypyridin-4-one (Deferiprone, DFP or Ferriprox); 2-deoxy-2-(N-carbamoylmethyl-[N′-2′-methyl-3′-hydroxypyridin-4′-one])-D-glucopyranose (Feralex-G), pyridoxal isonicotinyl hydrazone (P1H); 4,5-dihydro-2-(2,4-dihydroxyphenyl)-4-methylthiazole-4-carboxylic acid (GT56-252), 4-[3,5-bis(2-hydroxyphenyl)-[1,2,4]-triazol-1-yl]benzoic acid (ICL-670); N,N′-bis(o-hydroxybenzyl)ethylenediamine-N,N′-diacetic acid (HBED), 5-chloro-7-iodo-quinolin-8-ol (clioquinol), and derivatives, analogues, and combinations thereof.

It will be recognized that combinations of two or more of any of the foregoing metal chelators can be used in combination in the formulations of the invention. For example, in a particular embodiment of the invention, the formulation comprises a combination of DTPA and DEF. In another embodiment, the formulation comprises a to combination of EDTA, EGTA and DEF.

The amount of antibody present in the formulation is determined, for example, by taking into account the desired dose volumes and mode(s) of administration. In one embodiment of the invention, the concentration of the antibody in the formulation is between about 0.1 to about 250 mg of antibody per ml of liquid formulation. In one embodiment of the invention, the concentration of the antibody in the formulation is between about 1 to about 200 mg of antibody per ml of liquid formulation. In various embodiments, the concentration of the antibody in the formulation is between about 30 to about 140 mg per ml, between about 40 to about 120 mg/ml, between about 50 to about 110 mg/ml, or between about 60 to about 100 mg/ml. The formulation is especially suitable for large antibody dosages of more than 15 mg/ml. In various embodiments, the concentration of the antibody in the formulation is about 1, 10, 20, 30, 40, 50, 60, 70, 80, 90, 100, 110, 120, 130, 140, 150, 160, 170, 180, 190, 200, 210, 220, 230, 240 or 250 mg/ml. In a preferred embodiment, the concentration of the antibody is 50 mg/ml. In another preferred embodiment, the concentration of the antibody is 100 mg/ml. In a preferred embodiment, the concentration of the antibody is at least about 100 mg/ml, at least about 110 mg/ml or at least about 120 mg/ml.

In various embodiments of the invention, the concentration of the antibody in the formulation is about 0.1-250 mg/ml, 0.5-220 mg/ml, 1-210 mg/ml, about 5-200 mg/ml, about 10-195 mg/ml, about 15-190 mg/ml, about 20-185 mg/ml, about 25-180 mg/ml, about 30-175 mg/ml, about 35-170 mg/ml, about 40-165 mg/ml, about 45-160 mg/ml, about 50-155 mg/ml, about 55-150 mg/ml, about 60-145 mg/ml, about 65-140 mg/ml, about 70-135 mg/ml, about 75-130 mg/ml, about 80-125 mg/ml, about 85-120 mg/ml, about 90-115 mg/ml, about 95-110 mg/ml, about 95-105 mg/ml, or about 100 mg/ml. Ranges intermediate to the above recited concentrations, e.g., about 31-174 mg/ml, are also intended to be part of this invention. For example, ranges of values using a combination of any of the above recited values as upper and/or lower limits are intended to be included.

In one, the formulation provides an effective dose of 40 mg, 50 mg, 80 mg, 100 mg, or 200 mg per injection of the active ingredient, the antibody. In another embodiment, the formulation provides an effective dose which ranges from about 0.1 to 250 mg of antibody. If desired, the effective daily dose of the pharmaceutical formulation may be administered as two, three, four, five, six or more sub-doses to administered separately at appropriate intervals throughout the day, optionally, in unit dosage forms. In an embodiment of the invention, the dosage of the antibody in the formulation is between about 1 to about 200 mg. In an embodiment, the dosage of the antibody in the formulation is between about 30 and about 140 mg, between about 40 and about 120 mg, between about 50 and about 110 mg, between about 60 and about 100 mg, or between about 70 and about 90 mg. In one embodiment, the pharmaceutical composition includes the antibody at a dose of about 100 to about 200 mg. In a further embodiment, the composition includes the antibody at about 1, 10, 20, 30, 40, 50, 60, 70, 80, 90, 100, 110, 120, 130, 140, 150, 160, 170, 180, 190, 200, 210, 220, 230, 240 or 250 mg.

Ranges intermediate to the above recited dosages, e.g., about 2-139 mg, are also intended to be part of this invention. For example, ranges of values using a combination of any of the above recited values as upper and/or lower limits are intended to be included.

The compositions of this invention may be in a variety of forms. These include, for example, liquid, semi-solid and solid dosage forms, such as liquid solutions (e.g., injectable and infusible solutions), dispersions or suspensions, tablets, pills, powders, liposomes and suppositories. The preferred form depends on the intended mode of administration and therapeutic application. Typical preferred compositions are in the form of injectable or infusible solutions, such as compositions similar to those used for passive immunization of humans with other antibodies. The preferred mode of administration is parenteral (e.g., intravenous, subcutaneous, intraperitoneal, intramuscular). In a preferred embodiment, the antibody, or antigen-binding fragment thereof, is administered by subcutaneous injection.

Therapeutic compositions typically must be sterile and stable under the conditions of manufacture and storage. The composition can be formulated as a solution, microemulsion, dispersion, liposome, or other ordered structure suitable to high drug concentration. Sterile injectable solutions can be prepared by incorporating the active compound (i.e., antibody or antibody portion) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile, lyophilized powders for the preparation of sterile injectable solutions, the preferred methods of preparation are vacuum drying and spray-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

The antibodies and antibody-portions of the present invention can be administered by a variety of methods known in the art, although for many therapeutic applications, the preferred route/mode of administration is subcutaneous injection, intravenous injection or infusion. As will be appreciated by the skilled artisan, the route and/or mode of administration will vary depending upon the desired results. In certain embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

In certain embodiments, an antibody or antibody portion of the invention may be orally administered, for example, with an inert diluent or an assimilable edible carrier. The compound (and other ingredients, if desired) may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound of the invention by other than parenteral administration, it may be necessary to coat the compound with, or co-administer the compound with, a material to prevent its inactivation.

Supplementary active compounds can also be incorporated into the compositions. In certain embodiments, an antibody or antibody portion of the invention to is coformulated with and/or coadministered with one or more additional therapeutic agents that are useful for treating disorders in which IL-12 activity is detrimental. For example, an anti-hIL-12 antibody or antibody portion of the invention may be coformulated and/or coadministered with one or more additional antibodies that bind other targets (e.g., antibodies that bind other cytokines or that bind cell surface molecules). Furthermore, one or more antibodies of the invention may be used in combination with two or more of the foregoing therapeutic agents. Such combination therapies may advantageously utilize lower dosages of the administered therapeutic agents, thus avoiding possible toxicities or complications associated with the various monotherapies. It will be appreciated by the skilled practitioner that when the antibodies of the invention are used as part of a combination therapy, a lower dosage of antibody may be desirable than when the antibody alone is administered to a subject (e.g., a synergistic therapeutic effect may be achieved through the use of combination therapy which, in turn, permits use of a lower dose of the antibody to achieve the desired therapeutic effect).

Interleukin 12 plays a critical role in the pathology associated with a variety of diseases involving immune and inflammatory elements. These diseases include, but are not limited to, rheumatoid arthritis, osteoarthritis, juvenile chronic arthritis, Lyme arthritis, psoriatic arthritis, reactive arthritis, spondyloarthropathy, systemic lupus erythematosus, Crohn's disease, ulcerative colitis, inflammatory bowel disease, insulin dependent diabetes mellitus, thyroiditis, asthma, allergic diseases, psoriasis, dermatitis scleroderma, atopic dermatitis, graft versus host disease, organ transplant rejection, acute or chronic immune disease associated with organ transplantation, sarcoidosis, atherosclerosis, disseminated intravascular coagulation, Kawasaki's disease, Grave's disease, nephrotic syndrome, chronic fatigue syndrome, Wegener's granulomatosis, Henoch-Schoenlein purpurea, microscopic vasculitis of the kidneys, chronic active hepatitis, uveitis, septic shock, toxic shock syndrome, sepsis syndrome, cachexia, infectious diseases, parasitic diseases, acquired immunodeficiency syndrome, acute transverse myelitis, Huntington's chorea, Parkinson's disease, Alzheimer's disease, stroke, primary biliary cirrhosis, hemolytic anemia, malignancies, heart failure, myocardial infarction, Addison's disease, sporadic, polyglandular deficiency type I and polyglandular deficiency type II, Schmidt's syndrome, adult (acute) respiratory distress syndrome, alopecia, alopecia greata, seronegative arthopathy, arthropathy, Reiter's to disease, psoriatic arthropathy, ulcerative colitic arthropathy, enteropathic synovitis, chlamydia, yersinia and salmonella associated arthropathy, spondyloarthopathy, atheromatous disease/arteriosclerosis, atopic allergy, autoimmune bullous disease, pemphigus vulgaris, pemphigus foliaceus, pemphigoid, linear IgA disease, autoimmune haemolytic anaemia, Coombs positive haemolytic anaemia, acquired pernicious anaemia, juvenile pernicious anaemia, myalgic encephalitis/Royal Free Disease, chronic mucocutaneous candidiasis, giant cell arteritis, primary sclerosing hepatitis, cryptogenic autoimmune hepatitis, Acquired Immunodeficiency Disease Syndrome, Acquired Immunodeficiency Related Diseases, Hepatitis C, common varied immunodeficiency (common variable hypogammaglobulinaemia), dilated cardiomyopathy, female infertility, ovarian failure, premature ovarian failure, fibrotic lung disease, cryptogenic fibrosing alveolitis, post-inflammatory interstitial lung disease, interstitial pneumonitis, connective tissue disease associated interstitial lung disease, mixed connective tissue disease associated lung disease, systemic sclerosis associated interstitial lung disease, rheumatoid arthritis associated interstitial lung disease, systemic lupus erythematosus associated lung disease, dermatomyositis/polymyositis associated lung disease, Sjögren's disease associated lung disease, ankylosing spondylitis associated lung disease, vasculitic diffuse lung disease, haemosiderosis associated lung disease, drug-induced interstitial lung disease, radiation fibrosis, bronchiolitis obliterans, chronic eosinophilic pneumonia, lymphocytic infiltrative lung disease, postinfectious interstitial lung disease, gouty arthritis, autoimmune hepatitis, type-1 autoimmune hepatitis (classical autoimmune or lupoid hepatitis), type-2 autoimmune hepatitis (anti-LKM antibody hepatitis), autoimmune mediated hypoglycemia, type B insulin resistance with acanthosis nigricans, hypoparathyroidism, acute immune disease associated with organ transplantation, chronic immune disease associated with organ transplantation, osteoarthrosis, primary sclerosing cholangitis, idiopathic leucopenia, autoimmune neutropenia, renal disease NOS, glomerulonephritides, microscopic vasulitis of the kidneys, lyme disease, discoid lupus erythematosus, male infertility idiopathic or NOS, sperm autoimmunity, multiple sclerosis (all subtypes), insulin-dependent diabetes mellitus, sympathetic ophthalmia, pulmonary hypertension secondary to connective tissue disease, Goodpasture's syndrome, pulmonary manifestation of polyarteritis nodosa, acute rheumatic fever, rheumatoid spondylitis, Still's disease, systemic sclerosis, Takayasu's disease/arteritis, autoimmune thrombocytopenia, idiopathic to thrombocytopenia, autoimmune thyroid disease, hyperthyroidism, goitrous autoimmune hypothyroidism (Hashimoto's disease), atrophic autoimmune hypothyroidism, primary myxoedema, phacogenic uveitis, primary vasculitis and vitiligo. The human antibodies, and antibody portions of the invention can be used to treat autoimmune diseases, in particular those associated with inflammation, including, rheumatoid spondylitis, allergy, autoimmune diabetes, autoimmune uveitis.

Preferably, the antibodies of the invention or antigen-binding portions thereof, are used to treat rheumatoid arthritis, Crohn's disease, multiple sclerosis, insulin dependent diabetes mellitus and psoriasis, as described in more detail in section VII.

A human antibody, or antibody portion, of the invention also can be administered with one or more additional therapeutic agents useful in the treatment of autoimmune and inflammatory diseases.

Antibodies of the invention, or antigen binding portions thereof, can be used alone or in combination to treat such diseases. It should be understood that the IL-12 antibodies of the invention or antigen binding portion thereof can be used alone or in combination with an additional agent, e.g., a therapeutic agent, said additional agent being selected by the skilled artisan for its intended purpose. For example, the additional agent can be a therapeutic agent art-recognized as being useful to treat the disease or condition being treated by the antibody of the present invention. The additional agent also can be an agent which imparts a beneficial attribute to the therapeutic composition e.g., an agent which effects the viscosity of the composition.

It should further be understood that the combinations which are to be included within this invention are those combinations useful for their intended purpose. The agents set forth below are illustrative for purposes and not intended to be limited. The combinations which are part of this invention can be the antibodies of the present invention and at least one additional agent selected from the lists below. The combination can also include more than one additional agent, e.g., two or three additional agents if the combination is such that the formed composition can perform its intended function. Furthermore, additional agents described herein used in combination with an IL-12 antibody, are not limited to the disorder to which they are attributed for treatment.

Preferred combinations are non-steroidal anti-inflammatory drug(s) also referred to as NSAIDS which include drugs like ibuprofen. Other preferred combinations are corticosteroids including prednisolone; the well known side-effects of steroid use can be reduced or even eliminated by tapering the steroid dose required when treating patients in combination with the anti-p40 subunit of IL-12/IL-23 antibodies of this invention. Non-limiting examples of therapeutic agents for rheumatoid arthritis with which an antibody, or antibody portion, of the invention can be combined include the following: cytokine suppressive anti-inflammatory drug(s) (CSAIDs); antibodies to or antagonists of other human cytokines or growth factors, for example, TNF (including adalimumab/HUMIRA), LT, IL-1, IL-2, IL-6, IL-7, IL-8, IL-15, IL-16, IL-18, EMAP-II, GM-CSF, FGF, and PDGF. Antibodies of the invention, or antigen binding portions thereof, can be combined with antibodies to cell surface molecules such as CD2, CD3, CD4, CD8, CD25, CD28, CD30, CD40, CD45, CD69, CD80 (B7.1), CD86 (B7.2), CD90, or their ligands including CD154 (gp39 or CD40L).

Preferred combinations of therapeutic agents may interfere at different points in the autoimmune and subsequent inflammatory cascade; preferred examples include TNF antagonists like chimeric, humanized or human TNF antibodies, D2E7, (U.S. application Ser. No. 08/599,226 filed Feb. 9, 1996), cA2 (Remicade™), CDP 571, anti-TNF antibody fragments (e.g., CDP870), and soluble p55 or p75 TNF receptors, derivatives thereof, (p75TNFR1gG (Enbrel™) or p55TNFR1gG (Lenercept), soluble IL-13 receptor (sIL-13), and also TNFα converting enzyme (TACE) inhibitors; similarly IL-1 inhibitors (e.g., Interleukin-1-converting enzyme inhibitors, such as Vx740, or IL-1RA etc.) may be effective for the same reason. Other preferred combinations include Interleukin 11, anti-P7s and p-selectin glycoprotein ligand (PSGL). Yet another preferred combination are other key players of the autoimmune response which may act parallel to, dependent on or in concert with IL-12 function; especially preferred are IL-18 antagonists including IL-18 antibodies or soluble IL-18 receptors, or IL-18 binding proteins. It has been shown that IL-12 and IL-18 have overlapping but distinct functions and a combination of antagonists to both may be most effective. Yet another preferred combination are non-depleting anti-CD4 inhibitors. Yet other preferred combinations include antagonists of the co-stimulatory pathway CD80 (B7.1) or CD86 (B7.2) including antibodies, soluble receptors or antagonistic ligands.

Anti-p40 subunit of IL-12/IL-23 antibodies, or antigen binding portions thereof, may also be combined with agents, such as methotrexate, 6-MP, azathioprine sulphasalazine, mesalazine, olsalazine chloroquinine/hydroxychloroquine, pencillamine, to aurothiomalate (intramuscular and oral), azathioprine, cochicine, corticosteroids (oral, inhaled and local injection), beta-2 adrenoreceptor agonists (salbutamol, terbutaline, salmeteral), xanthines (theophylline, aminophylline), cromoglycate, nedocromil, ketotifen, ipratropium and oxitropium, cyclosporin, FK506, rapamycin, mycophenolate mofetil, leflunomide, NSAIDs, for example, ibuprofen, corticosteroids such as prednisolone, phosphodiesterase inhibitors, adensosine agonists, antithrombotic agents, complement inhibitors, adrenergic agents, agents which interfere with signaling by proinflammatory cytokines such as TNFα or IL-1 (e.g. IRAK, NIK, IKK, p38 or MAP kinase inhibitors), IL-1β converting enzyme inhibitors (e.g., Vx740), anti-P7s, p-selectin glycoprotein ligand (PSGL), TNFα converting enzyme (TACE) inhibitors, T-cell signaling inhibitors such as kinase inhibitors, metalloproteinase inhibitors, sulfasalazine, azathioprine, 6-mercaptopurines, angiotensin converting enzyme inhibitors, soluble cytokine receptors and derivatives thereof (e.g. soluble p55 or p75 TNF receptors and the derivatives p75TNFRIgG (Enbrel™) and p55TNFRIgG (Lenercept), sIL-1R1, sIL-1R11, sIL-6R, soluble IL-13 receptor (sIL-13)) and anti-inflammatory cytokines (e.g. IL-4, IL-10, IL-11, IL-13 and TGFβ). Preferred combinations include methotrexate or leflunomide and in moderate or severe rheumatoid arthritis cases, cyclosporine.

Non-limiting examples of therapeutic agents for inflammatory bowel disease with which an anti-IL-12 antibody, or antibody portion, can be combined include the following: budenoside; epidermal growth factor; corticosteroids; cyclosporin, sulfasalazine; aminosalicylates; 6-mercaptopurine; azathioprine; metronidazole; lipoxygenase inhibitors; mesalamine; olsalazine; balsalazide; antioxidants; thromboxane inhibitors; IL-1 receptor antagonists; anti-IL-1β monoclonal antibodies; anti-IL-6 monoclonal antibodies; growth factors; elastase inhibitors; pyridinyl-imidazole compounds; antibodies to or antagonists of other human cytokines or growth factors, for example, TNF (including adalimumab/HUMIRA), LT, IL-1, IL-2, IL-6, IL-7, IL-8, IL-15, IL-16, IL-18, EMAP-II, GM-CSF, FGF, and PDGF. Antibodies of the invention, or antigen binding portions thereof, can be combined with antibodies to cell surface molecules such as CD2, CD3, CD4, CD8, CD25, CD28, CD30, CD40, CD45, CD69, CD90 or their ligands. The antibodies of the invention, or antigen binding portions thereof, may also be combined with agents, such as methotrexate, cyclosporin, FK506, rapamycin, mycophenolate mofetil, leflunomide, NSAIDs, for example, ibuprofen, corticosteroids such as prednisolone, phosphodiesterase inhibitors, adenosine agonists, to antithrombotic agents, complement inhibitors, adrenergic agents, agents which interfere with signaling by proinflammatory cytokines such as TNFα or IL-1 (e.g. IRAK, NIK, IKK, p38 or MAP kinase inhibitors), IL-1β converting enzyme inhibitors (e.g., Vx740), anti-P7s, p-selectin glycoprotein ligand (PSGL), TNFα converting enzyme inhibitors, T-cell signaling inhibitors such as kinase inhibitors, metalloproteinase inhibitors, sulfasalazine, azathioprine, 6-mercaptopurines, angiotensin converting enzyme inhibitors, soluble cytokine receptors and derivatives thereof (e.g. soluble p55 or p75 TNF receptors, sIL-1R1, sIL-1R11, sIL-6R, soluble IL-13 receptor (sIL-13)) and anti-inflammatory cytokines (e.g. IL-4, IL-10, IL-11, IL-13 and TGFβ).

Preferred examples of therapeutic agents for Crohn's disease in which an antibody or an antigen binding portion can be combined include the following: TNF antagonists, for example, anti-TNF antibodies, D2E7 (adalimumab/HUMIRA), cA2 (Remicade™), CDP 571, anti-TNF antibody fragments (e.g., CDP870), TNFR-Ig constructs (p75TNFRIgG (Enbrel™) and p55TNFRIgG (Lenercept)), anti-P7s, p-selectin glycoprotein ligand (PSGL), soluble IL-13 receptor (sIL-13), and PDE4 inhibitors. Antibodies of the invention or antigen binding portions thereof, can be combined with corticosteroids, for example, budenoside and dexamethasone. Antibodies may also be combined with agents such as sulfasalazine, 5-aminosalicylic acid and olsalazine, and agents which interfere with synthesis or action of proinflammatory cytokines such as IL-1, for example, IL-1β converting enzyme inhibitors (e.g., Vx740) and IL-1ra. Antibodies or antigen binding portion thereof may also be used with T cell signaling inhibitors, for example, tyrosine kinase inhibitors 6-mercaptopurines. Antibodies or antigen binding portions thereof, can be combined with IL-11.

Non-limiting examples of therapeutic agents for multiple sclerosis with which an antibody, or antibody portion, can be combined include the following: corticosteroids; prednisolone; methylprednisolone; azathioprine; cyclophosphamide; cyclosporine; methotrexate; 4-aminopyridine; tizanidine; interferon-β1a (Avonex; Biogen); interferon-β1b (Betaseron; Chiron/Berlex); Copolymer 1 (Cop-1; Copaxone; Teva Pharmaceutical Industries, Inc.); hyperbaric oxygen; intravenous immunoglobulin; clabribine; antibodies to or antagonists of other human cytokines or growth factors, for example, TNF, LT, IL-1, IL-2, IL-6, IL-7, IL-8, IL-15, IL-16, IL-18, EMAP-II, GM-CSF, FGF, and PDGF. Antibodies of the invention, or antigen binding portions thereof, can be to combined with antibodies to cell surface molecules such as CD2, CD3, CD4, CD8, CD25, CD28, CD30, CD40, CD45, CD69, CD80, CD86, CD90 or their ligands. The antibodies of the invention, or antigen binding portions thereof, may also be combined with agents, such as methotrexate, cyclosporine, FK506, rapamycin, mycophenolate mofetil, leflunomide, NSAIDs, for example, ibuprofen, corticosteroids such as prednisolone, phosphodiesterase inhibitors, adensosine agonists, antithrombotic agents, complement inhibitors, adrenergic agents, agents which interfere with signaling by proinflammatory cytokines such as TNFα or IL-1 (e.g. IRAK, NIK, IKK, p38 or MAP kinase inhibitors), IL-10 converting enzyme inhibitors (e.g., Vx740), anti-P7s, p-selectin glycoprotein ligand (PSGL), TACE inhibitors, T-cell signaling inhibitors such as kinase inhibitors, metalloproteinase inhibitors, sulfasalazine, azathioprine, 6-mercaptopurines, angiotensin converting enzyme inhibitors, soluble cytokine receptors and derivatives thereof (e.g. soluble p55 or p75 TNF receptors, sIL-1R1, sIL-1R11, sIL-6R, soluble IL-13 receptor (sIL-13)) and anti-inflammatory cytokines (e.g. IL-4, IL-10, IL-13 and TGFβ).

Preferred examples of therapeutic agents for multiple sclerosis in which the antibody or antigen binding portion thereof can be combined to include interferon-β, for example, IFNβ1a and IFNβ1b; copaxone, corticosteroids, IL-1 inhibitors, TNF inhibitors, and antibodies to CD40 ligand and CD80.

An antibody, antibody portion, may be used in combination with other agents to treat skin conditions. For example, an antibody, antibody portion, or other p40 subunit of IL-12/IL-23 inhibitor of the invention is combined with PUVA therapy. PUVA is a combination of psoralen (P) and long-wave ultraviolet radiation (UVA) that is used to treat many different skin conditions. The antibodies, antibody portions, or other p40 subunit of IL-12/IL-23 inhibitors of the invention can also be combined with pimecrolimus. In another embodiment, the antibodies of the invention are used to treat psoriasis, wherein the antibodies are administered in combination with tacrolimus. In a further embodiment, tacrolimus and p40 subunit of IL-12/IL-23 inhibitors are administered in combination with methotrexate and/or cyclosporine. In still another embodiment, the p40 subunit of IL-12/IL-23 inhibitor of the invention is administered with excimer laser treatment for treating psoriasis.

The pharmaceutical compositions of the invention may include a “therapeutically effective amount” or a “prophylactically effective amount” of an antibody or antibody portion of the invention. A “therapeutically effective amount” refers to an amount to effective, at dosages and for periods of time necessary, to achieve the desired therapeutic result. A therapeutically effective amount of the antibody or antibody portion may vary according to factors such as the disease state, age, sex, and weight of the individual, and the ability of the antibody or antibody portion to elicit a desired response in the individual. A therapeutically effective amount is also one in which any toxic or detrimental effects of the antibody or antibody portion are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Dosage regimens may be adjusted to provide the optimum desired response (e.g., a therapeutic or prophylactic response). For example, a single bolus may be administered, several divided doses may be administered over time or the dose may be proportionally reduced or increased as indicated by the exigencies of the therapeutic situation.

It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic or prophylactic effect to be achieved, and (b) the limitations inherent in the art of compounding such an active compound for the treatment of sensitivity in individuals.

Treatment of psoriasis may be achieved by administration of a single dose amount (or more than one sub-doses totaling the dose amount) of a substance according to a single periodicity.

In one embodiment, a method of treating psoriasis in a subject comprises administering to the subject an antibody, or antigen-binding portion thereof, which is capable of binding to the p40 subunit of IL-12 and/or IL-23, according to a periodicity of about once every 4 weeks, thereby treating psoriasis in the subject.

In another embodiment, a method of treating psoriasis in a subject comprises administering to the subject an antibody, or antigen-binding portion thereof, which is capable of binding to the p40 subunit of IL-12 and/or IL-23, according to a periodicity of about once every 12 weeks, thereby treating psoriasis in the subject.

Thus, a single periodicity may be employed in a single treatment regimen. Alternatively, multiple periodicities may be employed in a single treatment regimen. For example, a first dose amount may be administered according to a first periodicity, and then the first dose amount or a second dose amount may be administered according to a second periodicity. Furthermore, the first dose amount or second dose amount administered according to a second periodicity may optionally be followed by a first, second, or third dose amount administered according to a third periodicity.

In one embodiment, an antibody, or antigen-binding portion thereof, which is capable of binding to the p40 subunit of IL-12 and/or IL-23 is administered to a subject as a first dose amount according to a periodicity, and is further administered to the subject as a second dose amount at the same periodicity.

In another embodiment, an antibody, or antigen-binding portion thereof, which is capable of binding to the p40 subunit of IL-12 and/or IL-23 is administered to a subject as a first dose amount according to a periodicity, and is further administered to the subject as a second dose amount according to a second periodicity.

In one embodiment, an antibody, or antigen-binding portion thereof, which is capable of binding to the p40 subunit of IL-12 and/or IL-23 is administered to a subject as a first dose amount according to a periodicity, and is further administered to the subject as a second dose amount according to a second periodicity, and is further administered to the subject as a first, second, or third dose amount according to a third periodicity.

The first dose amount of the antibody, or antigen-binding portion thereof, may be at least about 100 mg to about 200 mg, is at least about 100 mg, or is at least about 200 mg. The first dose amount of the antibody, or antigen-binding portion thereof, may be about 100 mg, about 110 mg, about 120 mg, about 130 mg, about 140 mg, about 150 mg, about 160 mg, about 170 mg, about 180 mg, about 190 mg, or about 200 mg. In one embodiment, the first dose amount is about 180-220 mg, 185-215 mg, 190-210 mg, or 195-205 mg. In one embodiment, the first dose amount is 200 mg. In one embodiment, the first dose amount is about 80-120 mg, 85-115 mg, 90-110 mg or 95-105 mg. In one embodiment, the first dose amount is 100 mg. It should be noted that doses intermediate to the above specified doses are also included herein, e.g., 105 mg, 127 mg, etc.

The second dose amount of the antibody, or antigen-binding portion thereof, may be the same as the first dose amount of the antibody, or antigen-binding portion thereof, or different than the first dose amount of the antibody, or antigen-binding portion thereof. The second dose amount of the antibody, or antigen-binding portion thereof, may be at least about 100 mg to about 200 mg, is at least about 200 mg, or is at least about 100 mg. Alternatively, the second dose amount of the antibody, or antigen-binding portion thereof, is about 40-60% (e.g., 40, 41, 42, 43, 44, 45, 46, 47, 48, 49, 50, 51, 52, 53, 54, 55, 56, 57, 58, 59 or 60%), e.g., about 50%, of the first dose amount of the antibody, or antigen-binding portion thereof, or antigen-binding portion thereof, or about 190-210% (e.g., 190, 191, 192, 193, 194, 195, 196, 197, 198, 199, 200, 201, 202, 203, 204, 205, 206, 207, 208, 209, 210%), e.g., about 200%, of the first dose amount of the antibody, or antigen-binding portion thereof. The second dose amount of the antibody, or antigen-binding portion thereof, may be about 100 mg, about 110 mg, about 120 mg, about 130 mg, about 140 mg, about 150 mg, about 160 mg, about 170 mg, about 180 mg, about 190 mg, or about 200 mg. In one embodiment, the second dose amount is about 80-120 mg, 85-115 mg, 90-110 mg or 95-105 mg. In one embodiment, the second dose amount is 100 mg. In another embodiment, the second dose amount is about 180-220 mg, 185-215 mg, 190-210 mg, or 195-205 mg. In one embodiment, the second dose amount is 200 mg. It should be noted that doses intermediate to the above specified doses are also included herein, e.g., 105 mg, 127 mg, etc.

The first and second periodicities of administration of the antibody, or antigen-binding portion thereof, may be about once a week, about once every other week, about once every four weeks. The second periodicity of administration of the antibody, or antigen-binding portion thereof, may be about once every 30-200 days.

The duration of the first periodicity may be about 12 weeks, about 8 weeks, about 4 weeks, about 2 weeks, or about 1 week.

The duration of the second periodicity may be about 60 weeks, about 44 weeks, about 12 weeks, about 4 weeks, about 2 weeks, or about 1 week.

The duration of a third periodicity may be, for example, about 4 weeks, about 12 to weeks, about 24 weeks, about 36 weeks, about 48 weeks or about 60 weeks.

Thus, in one aspect, a method of treating psoriasis in a subject comprises administering to the subject a first dose amount of an antibody, or antigen-binding portion thereof, which is capable of binding to the p40 subunit of IL-12 and/or IL-23; and a second dose amount that is about 40-60% of the first dose amount of the antibody, or antigen-binding portion thereof, according to a periodicity of about once every 12 weeks, thereby treating psoriasis in the subject.

In another aspect, a method of treating psoriasis in a subject comprises administering to the subject a first dose amount of an antibody, or antigen-binding portion thereof, which is capable of binding to the p40 subunit of IL-12 and/or IL-23, according to a first periodicity of about once every 4 weeks; and administering a second dose amount that is about 40-60% of the first dose amount of the antibody, or antigen-binding portion thereof, according to a second periodicity of about once every 4 weeks, thereby treating psoriasis in the subject.

In another aspect, a method of treating psoriasis in a subject comprises administering to the subject a first dose amount of an antibody, or antigen-binding portion thereof, which is capable of binding to the p40 subunit of IL-12 and/or IL-23, according to a first periodicity of about once every 4 weeks; and a second dose amount that is about 40-60% of the first dose amount of the antibody, or antigen-binding portion thereof, according to a second periodicity of about once every 4 weeks; and the second dose amount of the antibody, or antigen-binding portion thereof, according to a third periodicity of about once every 12 weeks, thereby treating psoriasis in the subject.

In one embodiment of the invention, a method of treating psoriasis in a subject comprises administering to the subject an antibody, or antigen-binding portion thereof, according to a dosing regimen as described in U.S. Ser. No. 12/881,902 (US 2011-0206680) and/or WO 2011/032148, the entire contents of which are expressly incorporated herein by reference. In one embodiment of the invention, a method of treating psoriasis in a subject comprises administering to the subject an antibody, or antigen-binding portion thereof, according to a dosing regimen as described in U.S. Pat. No. 7,776,331, the entire contents of which are expressly incorporated herein by reference. In one embodiment of the invention, a method of treating psoriasis in a subject comprises administering to the subject an antibody, or antigen-binding portion thereof, according to a dosing regimen as described in U.S. Ser. No. 12/402,342, which published as U.S. 2010-0028363 A1, the entire contents of which are expressly incorporated herein by reference.

In one embodiment, the second dose amount is administered to the subject upon a flare of psoriasis. In another embodiment, the second dose amount is administered to the subject prior to a flare of psoriasis.

The flare of psoriasis may be monitored by determining a subject's Psoriasis Area and Severity Index (PASI), e.g., PASI 100 response, PASI 90 response, PASI 75 response, PASI 50 response, the PASI response of a single body region, two body regions, three body regions, or four body regions, e.g., trunk, lower extremities, upper extremities, or head and neck. Alternatively, the flare of psoriasis may be monitored by determining a subject's Physician's Global Assessment (PGA) rating.

In one embodiment, the subject achieves or maintains a specific response to treatment. In one embodiment, the subject achieves or maintains at least a PASI 50 response. In one embodiment, the subject achieves or maintains at least a PASI 75 response. In one embodiment, the subject achieves or maintains at least a PASI 90 response. In one embodiment, the subject achieves or maintains at least a PASI 100 response. In one embodiment the PASI 50, 75, 90, or 100 response is achieved by about (e.g., at least about) week 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or 52 following treatment (e.g., following initial treatment, e.g., at week 0). In one embodiment, the PASI 50, 75, 90, or 100 response is maintained for about (e.g., at least about) 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52 weeks, e.g., following administration of a first dose amount at a first periodicity, or following administration of a first or second dose amount at a second periodicity, or following administration of a first, second or third dose amount according to a third periodicity. In one embodiment, the PASI 50, 75, 90 or 100 response is maintained, once achieved, throughout the duration of treatment.

In one embodiment, the subject achieves a PGA score of 0 or 1. In one embodiment the PGA score of 0 or 1 is achieved by about (e.g., at least about) week 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or 52 following treatment (e.g., following initial treatment, e.g., at week 0). In one to embodiment, the PGA score of 0 or 1 is maintained for about (e.g., at least about) 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52 weeks, e.g., following administration of a first dose amount at a first periodicity, or following administration of a first or second dose amount at a second periodicity, or following administration of a first, second or third dose amount according to a third periodicity. In one embodiment, the PGA score of 0 or 1 is maintained, once achieved, throughout the duration of treatment.

In one embodiment, the subject achieves a PGA score of 0, i.e., total clearance. In one embodiment the PGA score of 0 is achieved by about (e.g., at least about) week 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or 52 following treatment (e.g., following initial treatment, e.g., at week 0). In one embodiment, the PGA score of 0 is maintained for about (e.g., at least about) 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52 weeks, e.g., following administration of a first dose amount at a first periodicity, or following administration of a first or second dose amount at a second periodicity, or following administration of a first, second or third dose amount according to a third periodicity. In one embodiment, the PGA score of 0 is maintained, once achieved, throughout the duration of treatment.

A method of treating psoriasis in a subject or a population of subjects may comprise administering to the subject or each subject in the population an antibody, or antigen-binding portion thereof, which is capable of binding to the p40 subunit of IL-12 and/or IL-23, wherein the subject or a percentage of the population of subjects (e.g., at least about 10%, 15%, 20%, 25%, 30%, 35%, 40%, 45%, 50%, 55%, 60%, 65%, 70%, 75%, 80%, 85%, 90%, 95%, 99%, or 100% of a population of subjects) achieves at least a PASI 50 response by about week 12, 24, 36, 48, 52, or 60.

A method of treating psoriasis in a subject or a population of subjects may comprise administering to the subject or each subject in the population an antibody, or antigen-binding portion thereof, which is capable of binding to the p40 subunit of IL-12 and/or IL-23, wherein the subject or a percentage of the population of subjects (e.g., at least about 10%, 15%, 20%, 25%, 30%, 35%, 40%, 45%, 50%, 55%, 60%, 65%, 70%, to 75%, 80%, 85%, 90%, 95%, 99%, or 100% of a population of subjects) achieves at least a PASI 75 response by about week 12, 24, 36, 48, 52, or 60.

A method of treating psoriasis in a subject or a population of subjects may comprise administering to the subject or each subject in the population an antibody, or antigen-binding portion thereof, which is capable of binding to the p40 subunit of IL-12 and/or IL-23, wherein the subject or a percentage of the population of subjects (e.g., at least about 10%, 15%, 20%, 25%, 30%, 35%, 40%, 45%, 50%, 55%, 60%, 65%, 70%, 75%, 80%, 85%, 90%, 95%, 99%, or 100% of a population of subjects) achieves at least a PASI 90 response by about week 12, 24, 36, 48, 52, or 60.

A method of treating psoriasis in a subject or a population of subjects may comprise administering to the subject or each subject in the population an antibody, or antigen-binding portion thereof, which is capable of binding to the p40 subunit of IL-12 and/or IL-23, wherein the subject or a percentage of the population of subjects (e.g., at least about 10%, 15%, 20%, 25%, 30%, 35%, 40%, 45%, 50%, 55%, 60%, 65%, 70%, 75%, 80%, 85%, 90%, 95%, 99%, or 100% of a population of subjects) achieves at least a PASI 100 response by about week 12, 24, 36, 48, 52, or 60.

A method of treating psoriasis in a subject or a population of subjects may comprise administering to the subject or each subject in the population an antibody, or antigen-binding portion thereof, which is capable of binding to the p40 subunit of IL-12 and/or IL-23, wherein the subject or a percentage of the population of subjects (e.g., at least about 10%, 15%, 20%, 25%, 30%, 35%, 40%, 45%, 50%, 55%, 60%, 65%, 70%, 75%, 80%, 85%, 90%, 95%, 99%, or 100% of a population of subjects) achieves at least a PGA score of 0 or 1 by about week 12, 24, 36, 48, 52, or 60.

In one aspect, the subject or population of subjects treated achieves an improvement in a Dermatology Life Quality Index (DLQI) score or mean Dermatology Life Quality Index (DLQI) score of at least about −6.8, −6.9, −7.0, −8.0, −8.5, −9, −10, −10.5, −11, −12, −13, −14, −15, −16, −17, −18, −19, −20 or lower. An improvement in DLQI is a reduction in DLQI score, e.g., a reduction by at least about 6.8, 6.9, 7.0, 8.0, 8.5, 9, 10, 10.5, 11, 12, 13, 14, 15, 16, 17, 18, 19, 20 or more. Dermatology Life Quality Index (DLQI) is a patient-reported measure of the extent to which psoriasis impacts health-related quality of life. The DLQI yields a score ranging from 0 to 30, with a lower score indicating lower impact.

In certain embodiments, the subject achieves a clinically meaningful reduction in Dermatology Life Quality Index (DLQI) score. A clinically meaningful reduction in Dermatology Life Quality Index (DLQI) score may be, e.g., a decrease of greater than 5 points in DLQI score.

In another aspect, the subject or population of subjects treated achieves an improvement in a Short Form 36 Health Survey Physical Component Summary (PCS) score or mean Physical Component Summary (PCS) score of at least about 2, 3, 4, 5, 6, or more. An improvement in PCS is an increase in PCS score, e.g., an increase by at least about 2, 3, 4, 5, 6, or more.

In another aspect, the subject or population of subjects treated achieves an improvement in a Short Form 36 Health Survey Mental Component Summary (MCS) score or mean Mental Component Summary (MCS) score of at least about 3.5, 4, 4.5, 6, 6.5, 7, or more. An improvement in PCS is an increase in MCS score, e.g., an increase by at least about 3.5, 4, 4.5, 6, 6.5, 7, or more.

In another aspect, the subject or population of subjects treated achieves an improvement in a visual analog scale score or a mean visual analog scale score for psoriasis-related pain (VAS-Ps) of at least about −25, −26, −27, −28, −29, −30, −31, −32, −33, −34, −35, −40, −45, −50, or less. An improvement in VAS-Ps is a reduction in VAS-Ps score, e.g., a reduction by at least about 25, 26, 27, 28, 29, 30, 31, 32, 33, 34, 35, 40, 45, 50, or more.

In another aspect, the subject or population of subjects treated achieves an improvement in a visual analog scale score for psoriatic arthritis-related pain (VAS-PsA) or a mean visual analog scale score for psoriatic arthritis-related pain (VAS-PsA) of at least about −25, −26, −27, −28, −29, −30, −31, −32, −33, −34, −35, −40, −45, −50, or less. An improvement in VAS-PsA is a reduction in VAS-Ps score, e.g., a reduction by at least about 25, 26, 27, 28, 29, 30, 31, 32, 33, 34, 35, 40, 45, 50, or more.

In another aspect, the subject or population of subjects treated achieves a minimum clinically important difference (MCID) response rate in any one or more HRQOL outcomes including, e.g., DLQI, TAI, VAS-Ps, Vas-PsA, MCS and PCS of at least about 60%, 65%, 70%, 75%, 80%, 85%, 90%.

It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

### III. Uses of the Invention

The invention provides methods for inhibiting the activity of the p40 subunit of IL-12 and/or IL-23 in a subject suffering from a disorder in which the activity of the p40 subunit of IL-12 and/or IL-23 is detrimental. The invention also provides methods for treating psoriasis in a subject suffering from a disorder in which the activity of the p40 subunit of IL-12 and/or IL-23 is detrimental.

IL-12 has been implicated in the pathophysiology of a wide variety of disorders (Windhagen et al., (1995) J. Exp. Med. 182: 1985-1996; Morita et al. (1998) Arthritis and Rheumatism. 41: 306-314; Bucht et al., (1996) Clin. Exp. Immunol. 103: 347-367; Fais et al. (1994) J. Interferon Res. 14:235-238; Parronchi et al., (1997) Am. J. Path. 150:823-832; Monteleone et al., (1997) Gastroenterology. 112:1169-1178, and Berrebi et al., (1998) Am. J. Path 152:667-672; Parronchi et al (1997) Am. J. Path. 150:823-832). The invention provides methods for inhibiting the activity of the p40 subunit of IL-12 and/or IL-23 in a subject suffering from such a disorder, which method comprises administering to the subject an antibody or antibody portion of the invention such that the activity of the p40 subunit of IL-12 and/or IL-23 in the subject is inhibited. Preferably, the IL-12 and/or IL-23 is human IL-12 and/or IL-23 and the subject is a human subject. Alternatively, the subject can be a mammal expressing a IL-12 and/or IL-23 with which an antibody of the invention cross-reacts. Still further the subject can be a mammal into which has been introduced hIL-12 (e.g., by administration of hIL-12 or by expression of an hIL-12 transgene). An antibody of the invention can be administered to a human subject for therapeutic purposes (discussed further below). Moreover, an antibody of the invention can be administered to a non-human mammal expressing a IL-12 and/or IL-23 with which the antibody cross-reacts for veterinary purposes or as an animal model of human disease. Regarding the latter, such animal models may be useful for evaluating the therapeutic efficacy of antibodies of the invention (e.g., testing of dosages and time courses of administration).

As used herein, the phrase “a disorder in which the activity of the p40 subunit of to IL-12 and/or IL-23 is detrimental” is intended to include diseases and other disorders in which the presence of IL-12 and/or IL-23 in a subject suffering from the disorder has been shown to be or is suspected of being either responsible for the pathophysiology of the disorder or a factor that contributes to a worsening of the disorder. Accordingly, a disorder in which the activity of the p40 subunit of IL-12 and/or IL-23 is detrimental is a disorder in which inhibition of IL-12 and/or IL-23 activity is expected to alleviate the symptoms and/or progression of the disorder. Such disorders may be evidenced, for example, by an increase in the concentration of IL-12 and/or IL-23 in a biological fluid of a subject suffering from the disorder (e.g., an increase in the concentration of IL-12 and/or IL-23 in serum, plasma, synovial fluid, etc. of the subject), which can be detected, for example, using an anti-IL-12/IL-23 antibody as described above. There are numerous examples of disorders in which the activity of the p40 subunit of IL-12 and/or IL-23 is detrimental. In one embodiment, the antibodies or antigen binding portions thereof, can be used in therapy to treat the diseases or disorders described herein. In another embodiment, the antibodies or antigen binding portions thereof, can be used for the manufacture of a medicine for treating the diseases or disorders described herein.

Interleukin-12 (IL-12) and the related cytokine IL-23 have been implicated as key mediators in psoriasis. Psoriasis involves acute and chronic skin lesions that are associated with a TH1-type cytokine expression profile (Hamid et al. (1996) J. Allergy Clin. Immunol. 1:225-231; Turka et al. (1995) Mol. Med. 1:690-699). Both IL-12 and IL-23 contribute to the development of the type 1T helper cell (Th1) immune response in psoriasis. Moreover, the IL-12 p40 and IL-23 p40 messenger RNA is overexpressed in psoriatic skin lesions. Accordingly, the antibodies or antigen binding portions thereof of the invention may serve to alleviate chronic skin disorders such psoriasis.

In one embodiment, the invention provides a method for treating psoriasis. Treatment for psoriasis often includes a topical corticosteroids, vitamin D analogs, and topical or oral retinoids, or combinations thereof. In one embodiment, an IL-12 and/or IL-23 antibody is administered in combination with or the presence of one of these common treatments. Additional therapeutic agents which can be combined with the IL-12 and/or IL-23 antibody for treatment of psoriasis are described in more detail below.

The diagnosis of psoriasis is usually based on the appearance of the skin. Additionally a skin biopsy, or scraping and culture of skin patches may be needed to rule out other skin disorders. An x-ray may be used to check for psoriatic arthritis if joint pain is present and persistent.

Improvements in psoriasis in a subject can be monitored by the subject's Psoriasis Area and Severity Index Score (PASI). The method for determining the PASI has been described in Fredriksson and Pettersson (1978) Dermatologica 157:238 and Marks et al. (1989) Arch Dermatol 125:235. Briefly, the index is based on evaluation of four anatomic sites, including the head, upper extremities, trunk, and lower extremities, for erythema, induration, and desquamation using a 5 point scale (0=no symptoms; 1=slight; 2=moderate; 3=marked; 4=very marked). Based on the extent of lesions in a given anatomic site, the area affected is assigned a numerical value (0=0; 1=<10%; 2=10-29%; 3=30-49%; 4=50-69%; 5=70=89%; 6=90-100%). The PASI score is then calculated, wherein the possible range of PASI score is 0.0 to 72.0 with the highest score representing complete erythroderma of the severest degree.

In one embodiment of the invention, an IL-12 and/or IL-23 antibody is used for the treatment of psoriasis, including chronic plaque psoriasis, guttate psoriasis, inverse psoriasis, pustular psoriasis, pemphigus vulgaris, erythrodermic psoriasis, psoriasis associated with inflammatory bowel disease (IBD), and psoriasis associated with rheumatoid arthritis (RA). In another embodiment, an IL-12 and/or IL-23 antibody, such as J695, is used to treat subjects who have psoriasis in combination with psoriasis. Specific types of psoriasis included in the treatment methods of the invention are described in detail below:

a. Chronic Plaque Psoriasis

Chronic plaque psoriasis (also referred to as psoriasis vulgaris) is the most common form of psoriasis. Chronic plaque psoriasis is characterized by raised reddened patches of skin, ranging from coin-sized to much larger. In chronic plaque psoriasis, the plaques may be single or multiple, they may vary in size from a few millimeters to several centimeters. The plaques are usually red with a scaly surface, and reflect light when gently scratched, creating a “silvery” effect. Lesions (which are often symmetrical) from chronic plaque psoriasis occur all over body, but with predilection for extensor surfaces, including the knees, elbows, lumbosacral regions, scalp, and nails. Occasionally chronic plaque psoriasis can occur on the penis, vulva and flexures, but scaling is usually absent. Diagnosis of patients with chronic plaque psoriasis is usually based on the clinical features described above. In particular, the distribution, color and typical silvery scaling of the lesion in chronic plaque psoriasis are characteristic of to chronic plaque psoriasis.

b. Guttate Psoriasis

Guttate psoriasis refers to a form of psoriasis with characteristic water drop shaped scaly plaques. Flares of guttate psoriasis generally follow an infection, most notably a streptococcal throat infection. Diagnosis of guttate psoriasis is usually based on the appearance of the skin, and the fact that there is often a history of recent sore throat.

c. Inverse Psoriasis

Inverse psoriasis is a form of psoriasis in which the patient has smooth, usually moist areas of skin that are red and inflammed, which is unlike the scaling associated with plaque psoriasis. Inverse psoriasis is also referred to as intertiginous psoriasis or flexural psoriasis. Inverse psoriasis occurs mostly in the armpits, groin, under the breasts and in other skin folds around the genitals and buttocks, and, as a result of the locations of presentation, rubbing and sweating can irriate the affected areas.

d. Pustular Psoriasis

Pustular psoriasis, also referred to as palmar plantar psoriasis, is a form of psoriasis that causes pus-filled blisters that vary in size and location, but often occur on the hands and feet. The blisters may be localized, or spread over large areas of the body. Pustular psoriasis can be both tender and painful, can cause fevers.

e. Other Psoriasis Disorders

Other examples of psoriatic disorders which can be treated with the IL-12 and/or IL-23 antibody include erythrodermic psoriasis, vulgaris, psoriasis associated with IBD, and psoriasis associated with arthritis, including rheumatoid arthritis.

The use of the antibodies and antibody portions of the invention in the treatment of a few additional non-limiting specific disorders is discussed further below.

### A. Rheumatoid Arthritis:

Interleukin-12 has been implicated in playing a role in inflammatory diseases such as rheumatoid arthritis. Inducible IL-12p40 message has been detected in synovia from rheumatoid arthritis patients and IL-12 has been shown to be present in the synovial fluids from patients with rheumatoid arthritis (see e.g., Morita et al., (1998) Arthritis and Rheumatism 41: 306-314). IL-12 positive cells have been found to be to present in the sublining layer of the rheumatoid arthritis synovium. The human antibodies, and antibody portions of the invention can be used to treat, for example, rheumatoid arthritis, juvenile rheumatoid arthritis, Lyme arthritis, rheumatoid spondylitis, osteoarthritis and gouty arthritis. Typically, the antibody, or antibody portion, is administered systemically, although for certain disorders, local administration of the antibody or antibody portion may be beneficial. An antibody, or antibody portion, of the invention also can be administered with one or more additional therapeutic agents useful in the treatment of autoimmune diseases.

In the collagen induced arthritis (CIA) murine model for rheumatoid arthritis, treatment of mice with an anti-IL-12 mAb (rat anti-mouse IL-12 monoclonal antibody, C17.15) prior to arthritis profoundly suppressed the onset, and reduced the incidence and severity of disease. Treatment with the anti-IL-12 mAb early after onset of arthritis reduced severity, but later treatment of the mice with the anti-IL-12 mAb after the onset of disease had minimal effect on disease severity.

### B. Crohn's Disease

Interleukin-12 also plays a role in the inflammatory bowel disease, Crohn's disease. Increased expression of IFN-γ and IL-12 occurs in the intestinal mucosa of patients with Crohn's disease (see e.g., Fais et al., (1994) J. Interferon Res. 14: 235-238; Parronchi et al., (1997) Amer. J. Pathol. 150: 823-832; Monteleone et al., (1997) Gastroenterology 112: 1169-1178; Berrebi et al., (1998) Amer. J. Pathol. 152: 667-672). Anti-IL-12 antibodies have been shown to suppress disease in mouse models of colitis, e.g., TNBS induced colitis IL-2 knockout mice, and recently in IL-10 knock-out mice. Accordingly, the antibodies, and antibody portions, of the invention, can be used in the treatment of inflammatory bowel diseases.

### C. Multiple Sclerosis

Interleukin-12 has been implicated as a key mediator of multiple sclerosis. Expression of the inducible IL-12 p40 message or IL-12 itself can be demonstrated in lesions of patients with multiple sclerosis (Windhagen et al., (1995) J. Exp. Med. 182: 1985-1996, Drulovic et al., (1997) J. Neurol. Sci. 147: 145-150). Chronic progressive patients with multiple sclerosis have elevated circulating levels of IL-12. Investigations to with T-cells and antigen presenting cells (APCs) from patients with multiple sclerosis revealed a self-perpetuating series of immune interactions as the basis of progressive multiple sclerosis leading to a Th1-type immune response. Increased secretion of IFN-γ from the T cells led to increased IL-12 production by APCs, which perpetuated the cycle leading to a chronic state of a Th1-type immune activation and disease (Balashov et al., (1997) Proc. Natl. Acad. Sci. 94: 599-603). The role of IL-12 in multiple sclerosis has been investigated using mouse and rat experimental allergic encephalomyelitis (EAE) models of multiple sclerosis. In a relapsing-remitting EAE model of multiple sclerosis in mice, pretreatment with anti-IL-12 mAb delayed paralysis and reduced clinical scores. Treatment with anti-IL-12 mAb at the peak of paralysis or during the subsequent remission period reduced clinical scores. Accordingly, the antibodies or antigen binding portions thereof of the invention may serve to alleviate symptoms associated with multiple sclerosis in humans.

### D. Insulin-Dependent Diabetes Mellitus

Interleukin-12 has been implicated as an important mediator of insulin-dependent diabetes mellitus (IDDM). IDDM was induced in NOD mice by administration of IL-12, and anti-IL-12 antibodies were protective in an adoptive transfer model of IDDM. Early onset IDDM patients often experience a so-called “honeymoon period” during which some residual islet cell function is maintained. These residual islet cells produce insulin and regulate blood glucose levels better than administered insulin. Treatment of these early onset patients with an anti-IL-12 antibody may prevent further destruction of islet cells, thereby maintaining an endogenous source of insulin.

The contents of all cited references, including literature references, issued patents, and published patent applications, as cited throughout this application are hereby expressly incorporated herein by reference. It should further be understood that the contents of all the tables attached hereto as well as the entire contents of U.S. Pat. No. 6,914,128; U.S. Pat. No. 7,504,485 (issued Mar. 17, 2009), U.S. Pat. No. 7,776,331 (issued Aug. 17, 2010), U.S. Patent Application Publication No. 20100028363 (filed Mar. 11, 2009), U.S. Patent Application Publication No. 20100172862 (filed Nov. 24, 2010), U.S. Patent Application Publication No. 20110206680 (filed Sep. 14, 2010 and U.S. patent application Ser. No. 13/267,658 (filed Oct. 6, 2011) are expressly incorporated herein by reference.

The present invention is further illustrated by the following examples which should not be construed as limiting in any way.

### EXAMPLES

### Example 1

### Efficacy of the Fully Human IL-12/IL-23 Monoclonal Antibody, J695, In the Treatment of Moderate to Severe Plaque Psoriasis

J695 is a fully human antibody against interleukin-12 (IL-12) and IL-23. It binds with great affinity to the p40 subunit common to both IL-12 and IL-23, both validated targets in the treatment of psoriasis (Ps).

The objective of the following study was to evaluate the efficacy of subcutaneous injections of J695 in the treatment of patients with moderate to severe plaque Ps.

Adult patients with Ps affecting ≧10% body surface area (BSA) and a Psoriasis Area and Severity Index (PASI) score ≧12 at baseline were eligible for this 12-week, double-blind, placebo-controlled study. Patients were randomized to 1 of 6 arms: 1) 100-mg J695 every other week (eow) for 12 weeks; 2) one 200-mg J695 dose at Week 0; 3) 200-mg J695 every week for 4 weeks; 4) 200-mg J695 eow for 12 weeks; 5) 200-mg J695 every week for 12 weeks; or 6) placebo. Primary endpoint was a ≧PASI75 response at Week 12. Other efficacy assessments included the PASI50 and Physician's Global Assessment (PGA). Patients who met the primary endpoint entered a 36-week blinded/retreatment phase and were monitored for time to loss of response.

A total of 180 patients enrolled in the study, 30 in each arm. Baseline characteristics were similar between arms and indicative of moderate to severe Ps (all mean values except % male): age, 46 yrs, 74% male; 21 yrs duration of Ps; PASI 19; and 25% BSA affected. At Week 12, the percentages of patients achieving ≧PASI75 were statistically significantly greater for patients in each of the 5 J695 arms vs. placebo (93%, 63%, 90%, 93%, 90%, vs. 3%, respectively, p<0.001, ITT). In addition, the percentages of patients achieving ≧PASI50 were statistically significantly greater for patients in each of the 5 J695 arms vs. placebo (100%, 77%, 97%, 97%, and 100%, vs. 17%, p<0.001). The mean percentage decreases (improvements) in PASI at Week 12 were 90%, 70%, 92%, 92%, and 90%, respectively, in the J695 arms, and 26% for placebo. Similarly, the percentages of patients with a PGA of Clear/Minimal were 83%, 50%, 73%, 87% and 87%, respectively, in the J695 arms, and 3% for placebo.

In conclusion, J695 was significantly more efficacious than placebo in the treatment of moderate to severe plaque psoriasis.

### Example 2

### Safety and Efficacy of the Fully Human IL-12/-23 Monoclonal Antibody, J695, in the Treatment of Moderate to Severe Plaque Psoriasis

J695 is a fully human antibody against interleukin 12 (IL-12) and IL-23. It binds with great affinity to the p40 subunit common to both IL-12 and IL-23, validated targets in the treatment of psoriasis (Ps). The objective of this Phase II study was to investigate the efficacy and safety of subcutaneous injections of J695 in the treatment of moderate to severe plaque Ps.

Adults with Ps affecting ≧10% body surface area (BSA) and a PASI score ≧12 were eligible for this 12-wk, double-blind, placebo-controlled study. Patients were randomized to 1 of 6 arms: 1) 100-mg J695 every other week (eow) for 12 wks; 2) one 200-mg J695 dose at Wk 0; 3) 200-mg J695 every wk for 4 wks; 4) 200-mg J695 eow for 12 wks; 5) 200-mg J695 every wk for 12 wks; or 6) placebo. The primary endpoint was a ≧PASI75 response at Wk 12. Patients who met the primary endpoint entered a 36-wk blinded/retreatment phase and were monitored for time to loss of response. All patients were evaluated for safety through Wk 54.

180 patients enrolled, 30 in each arm. Baseline characteristics were similar between arms (mean values presented except % male): age, 46 yrs, 74% male; 21 yrs duration of Ps; PASI=19; and 25% BSA affected. At Wk 12, the % s of patients with ≧PASI75 were statistically significantly greater in each of the 5 J695 arms vs. placebo (93%, 63%, 90%, 93%, 90%, vs. 3%, respectively, p<0.001, ITT). During the 12-wk, DB phase, infectious AEs for the J695 groups ranged from 23-43% and for the placebo group was 23%, with the most common being nasopharyngitis (7-17% for J695; 3% for placebo). There were no statistically significant differences between arms. No serious infectious AEs were reported, and no deaths occurred.

In conclusion, J695 was significantly more efficacious than placebo in the treatment of moderate to severe plaque Ps, and appears to have a favorable safety profile.

### Example 3

### Maintenance of Response with the Fully Human IL-12/-23 Monoclonal Antibody, J695, in the Treatment of Moderate to Severe Plaque Psoriasis

The efficacy and safety of J695 was evaluated in a 12-week, Phase II, randomized controlled trial and 36-week follow-up phase. The objective of the following example was to analyze maintenance of response following discontinuation of therapy during the second 12 weeks of this Phase II study of subcutaneous injections of J695 in the treatment of moderate to severe plaque Ps.

Adults with Ps affecting ≧10% body surface area (BSA) and a PASI score ≧12 were eligible for this 12-week, double-blind, placebo-controlled study. Patients were randomized to 1 of 6 arms:

1) 100-mg J695 every other week (eow) for 12 wks;

2) one 200-mg J695 dose at wk 0;

3) 200-mg J695 every wk for 4 wks;

4) 200-mg J695 eow for 12 wks;

5) 200-mg J695 every wk for 12 wks; or

6) placebo.

The primary endpoint was a ≧PASI75 response at Week 12. Patients who met the primary endpoint entered a 36-week blinded/retreatment phase. Treatment with study drug was discontinued, and patients were monitored for time to loss of response (a decrease in PASI score, any time during the 36-week follow-up period, to <PASI 50). Maintenance of PASI response was evaluated through Week 24.

A total of 180 patients enrolled, 30 in each arm. Baseline characteristics were similar between arms (mean values presented except % male): age, 46 years, 74% male; 21 years duration of Ps; PASI=19; and 25% BSA affected.

At Week 12, the percentages of patients with ≧PASI75 were statistically significantly greater in each of the 5 J695 arms vs. placebo (Table 2). At Week 24, substantial percentages of PASI 75 responders in the active treatments arms had maintained at least a PASI 50 response.

TABLE 2
24-Week Efficacy of J695

| | | Maintenance of |
|---|---|---|
| | | PASI Response: |
| | ≧PASI75 at Wk 12 | Wk 24 vs. Wk 12 |
| 100 mg eow for 12 wks | 28/30 (93%)* | 24/28 (86%) |
| 200 mg, one dose | 19/30 (63%)* | 15/19 (79%) |
| 200-mg every wk for 4 wks | 27/30 (90%)* | 23/27 (85%) |
| 200-mg eow for 12 wks | 28/30 (93%)* | 26/28 (93%) |
| 200-mg every wk for 12 wks | 27/30 (90%)* | 26/27 (96%) |
| Placebo | 1/30 (3%) | — |

*p < 0.001 vs. placebo, NRI.

In conclusion, J695 was significantly more efficacious than placebo in the treatment of moderate to severe plaque Ps. Substantial percentages of PASI 75 responders maintained these responses at Week 24, following discontinuation of active therapy.

### Example 4

### Safety and Efficacy of J695, a Fully Human IL-12/-23 Monoclonal Antibody, in the Treatment of Moderate to Severe Chronic Plaque Psoriasis

The objective of the following example was to demonstrate the efficacy and safety of a range of doses of a human IL-12/23 monoclonal antibody (J695) compared with placebo in the treatment of patients with clinically stable moderate to severe chronic plaque psoriasis.

### I. Materials and Methods

A. Study Design:

The following study was a 12-week, multicenter, randomised, double-blind, phase II, placebo-controlled trial that was conducted at 24 centers in the United States (16 sites) and Canada (8 sites). J695 (Abbott Laboratories, Abbott Park, Ill.) is a human monoclonal antibody with genetically engineered complementarity-determining regions that have high affinity for the IL-12/23 p40 subunit protein. Patients were randomised in a 1:1:1:1:1:1 ratio to receive 1 of 6 treatments: 200 mg of J695, 1 dose at week 0 (200 mg×1); 100 mg of J695 every other week (eow) for 12 weeks (100 mg eow); 200 mg of J695 weekly for the first 4 weeks (200 mg×4); 200 mg of J695 eow for 12 weeks (200 mg eow); 200 mg of J695 weekly for 12 weeks (200 mg to weekly); or placebo. After week 12, all patients who achieved at least a 75% reduction in psoriasis area and severity index (PASI 75) response continued into a 36-week blinded observation/retreatment phase.

B. Patients:

Patients were ≧18 years of age and had a clinical diagnosis of psoriasis for at least 6 months (determined by patient interview and confirmation of diagnosis through physical examination by the investigator), stable plaque psoriasis for at least 2 months before screening and at baseline visits as determined by subject interview, moderate to severe plaque psoriasis defined by ≧10% body surface area (BSA) involvement at the baseline visit, a PASI score of ≧12 at the baseline visit, and a physician's global assessment (PGA) of at least moderate disease at the baseline visit. Patients were ineligible if they had previous exposure to systemic or biologic anti-IL-12 therapy; nonplaque psoriasis; inability to discontinue the following therapies before the baseline visit: topical psoriasis therapies at least 2 weeks before, ultraviolet B light phototherapy at least 2 weeks before, psoralen-ultraviolet-light phototherapy at least 4 weeks before, systemic therapies at least 4 weeks before, and biologic therapies at least 12 weeks before; required intake of oral or injectable corticosteroids during the study (inhaled corticosteroids for stable medical conditions were allowed); an exacerbation of asthma requiring hospitalization in the 10 years prior to screening; an infection or risk factors for severe infection; a history of malignancies other than successfully treated basal cell carcinoma (patients with a history of squamous cell carcinoma were excluded) or cervical carcinoma in situ; or a history of major immunologic reaction (eg, serum sickness or anaphylactoid reaction) to an immunoglobulin G-containing agent (e.g., intravenous gamma globulin, a fusion protein, or monoclonal antibody).

Patients were allowed to continue treatment with medicated shampoos that did not contain corticosteroids, bland (without beta- or alpha-hydroxy acids) emollients, or Class VI or VII low-potency topical corticosteroids on their palms, soles, face, inframammary area, and groin area during the course of the study. Application of these topical psoriasis therapies was not to occur within 24 hours of a study visit. Vaccination with a live viral agent was not allowed within 1 month prior to dosing with J695, during the study, or for 1 month after the last dose of study drug was administered.

Occurrence of any of the following clinically significant abnormal laboratory results led to immediate withdrawal of a patient from the study: aspartate transaminase or alanine transaminase >5 times the upper limit of normal; serum total bilirubin >3 times the upper limit of normal; serum creatinine >3 times the upper limit of normal; creatine phosphokinase >5 times the upper limit of normal; hemoglobin <8 g/dL; white blood cell count <2×10⁹/L; or platelet count <75×10⁹/L.

C. Efficacy Assessments:

The primary efficacy assessment was the percentage of patients achieving a PASI 75 response at week 12, defined as at least a 75% reduction in PASI score relative to the baseline score. PASI is a measure of the severity of psoriatic lesions (in terms of erythema, induration, and desquamation) and the extent of BSA involvement. The PASI score ranges from 0 (no psoriasis) to 72 (severe disease) (Fredriksson T, Pettersson U. Dermatologica 1978; 157: 238-44). Other efficacy measures included the percentage of patients who achieved at least PASI 75 at weeks 1, 2, 4, and 8; the percentage of patients who achieved at least PASI 50 or PASI 90 at weeks 1, 2, 4, 8, and 12; and the percentage of patients who attained a PGA of clear or minimal at week 12 and at weeks 1, 2, 4, and 8. The PGA measures the severity of disease on a 6-point scale, which ranges from 0 (no disease, or clear) to 5 (very severe) (Ko H—S. Clinical trial design in psoriasis. Presented at: 49th Meeting of the Dermatologic and Ophthalmologic Advisory Committee; Mar. 20, 1998; Bethesda, Md.).

D. Safety Assessments:

Adverse events, laboratory data, and vital signs were assessed throughout the study. Patients were closely monitored for signs of infection, malignancy, and immunologic reaction. Treatment-emergent AEs were defined as those events that occurred between week 0 and the earlier of 45 days after the last nonmissing study drug dose or 1 day prior to the first retreatment dose (for those patients continuing on to the 36-week trial).

E. Statistical analysis: The sample size was calculated using nQuery Advisor® 4.0 (Statistical Solutions, Saugus, Mass.). With the assumption that 15% of the patients in the placebo group would achieve a PASI 75 response at week 12, the study designers determined that a sample size of 26 in each dosage group would be adequate to detect at least a 45% difference from a treated group using the Fisher exact test with 90% power at a 0.05 2-sided significance level. The study was designed to enroll approximately 180 patients, with 30 patients in each group.

The intention-to-treat population included all patients who were randomised at week 0 and received at least 1 injection of study drug; this population was used for the efficacy analyses. All tests were performed at a=0.05. Nonresponder imputation was used for all efficacy analyses; any patient with a missing PASI or PGA score at any visit was considered a nonresponder at that visit. To assess the impact of the missing data, sensitivity analyses of week-12 data were completed using the last-observation-carried-forward method. The primary analysis of PASI 75 response at week 12 was performed using the following sequential order to adjust for multiplicity: 200 mg weekly versus placebo, 200 mg eow versus placebo, 100 mg eow versus placebo, 200 mg×4 versus placebo, and 200 mg×1 versus placebo. The treatment difference between each J695 treatment group and the placebo group for mean percentage change in PASI score was assessed using analysis of variance, with baseline PASI score and treatment group as factors. The safety analyses were conducted using the safety population, which included all patients who received at least 1 injection of study drug.

### II. Results

A. Patients:

A total of 180 patients were enrolled and randomised to 1 of the 6 treatment groups. The majority of patients (76.7% of placebo-treated patients and 98% of all J695 treatment group patients) completed the 12-week portion of the study.

Patients were well balanced across treatment groups with respect to demographic characteristics and disease activity. Patients were predominantly male (74.4%) and white (92.2%). Mean BSA involvement was 25% and mean PASI score was 18.8.

B. Efficacy:

The percentage of patients achieving the primary endpoint of PASI 75 response at week 12 was statistically significantly greater (p<0.001) in all of the J695 treatment groups (200 mg×1: 63.3%, 19 of 30; 100 mg eow: 93.3%, 28 of 30; 200 mg×4: 90.0%, 27 of 30; 200 mg eow: 93.3%, 28 of 30; 200 mg weekly: 90.0%, 27 of 30) compared with placebo (3.3%, 1 of 30). For the relatively short duration of this trial, PASI 75 responses in all J695 treatment groups were similar with the exception of the 200 mg×1 treatment group.

A subgroup analysis by demographics (gender, age, race, and weight), baseline disease characteristics (history of psoriatic arthritis, BSA, and PASI score), and baseline therapy for psoriasis within 12 months of receiving study treatment (systemic biologic and nonbiologic, topical, and phototherapy) demonstrated that J695-treated patients within the various subgroups consistently achieved high levels of PASI 75 response at to week 12.

Nearly 100% of the higher J695 dosage groups attained at least a PASI 50 response by week 12 (200 mg×1: 76.7%, 23 of 30; 100 mg eow: 100.0%, 30 of 30; 200 mg×4: 96.7%, 29 of 30; 200 mg eow: 96.7%, 29 of 30; 200 mg weekly: 100.0%, 30 of 30; placebo: 16.7%, 5 of 30; p<0.001 for each comparison with placebo). The percentage of patients achieving at least a PASI 90 response at week 12 was statistically significantly greater (p<0.001) in all but 1 (200 mg×1) of the J695 treatment groups when compared with placebo, as follows: 200 mg×1: 16.7%, 5 of 30; 100 mg eow: 53.3%, 16 of 30; 200 mg×4: 63.3%, 19 of 30; 200 mg eow: 76.6%, 23 of 30; 200 mg weekly: 53.3%, 16 of 30; and placebo: 0%, 0 of 30. In addition, by week 12, significantly more (p<0.001) patients in all J695 treatment groups had attained a clear or minimal PGA rating compared with patients in the placebo group, as follows: 200 mg×1: 50.0%, 15 of 30; 100 mg eow: 83.3%, 25 of 30; 200 mg×4: 73.3%, 22 of 30; 200 mg eow: 86.7%, 26 of 30; 200 mg weekly: 86.7%, 26 of 30; versus placebo: 3.3%, 1 of 30.

The percentage of patients achieving the primary endpoint of PASI 100 response at week 12 was statistically significantly greater (p<0.001) in the following J695 treatment groups (200 mg eow: 46.7%, 14 of 30; 200 mg weekly: 36.7%, 11 of 30) compared with placebo (0%, 0 of 30).

Response to J695 was rapid. The mean percentage improvement in PASI scores from baseline increased over time for all J695 treatment groups and were statistically significantly greater for each J695 treatment group compared with placebo at each time point (p<0.001, except for the 100 mg eow group at week 1, p=0.023).

C. Safety:

J695 therapy was generally well tolerated. One (0.7%) patient treated with J695 discontinued the study owing to a localized skin discoloration; 2 (6.7%) patients treated with placebo discontinued the study, 1 for psoriatic arthropathy and 1 for ovarian cancer. Two (1.1%) patients experienced serious adverse effects (AEs); 1 placebo-treated patient was diagnosed with ovarian cancer on day 37, and 1 J695-treated patient (200 mg×1) was diagnosed with costochondritis on day 10. No patients experienced myocardial or cerebral infarctions, and there were no deaths.

Patients receiving any dose of J695 were significantly (p=0.033) more likely than patients receiving placebo to experience an AE at least possibly related to study drug (J695: 36.0%, 54 of 150; placebo: 10.0%, 3 of 30); most of these AEs were related to the injection site (injection-site reaction, erythema, pruritus, or irritation).

Most AEs were mild (mild AEs occurred in 46.0% [69 of 150] of J695-treated patients and 30.0% [9 of 30] placebo-treated patients). The most common AE was injection-site reaction, occurring in 16.7% (25 of 150) of patients treated with any dose of J695 (no reported injection-site reactions for placebo-treated patients; p=0.028). There were no statistically significant differences between the incidences of other AEs in the J695-treated patients compared with placebo-treated patients. The next most frequently reported AEs were nasopharyngitis and upper respiratory tract infection.

Infectious AEs were reported by 32.8% (59 of 180) of all patients (placebo: 23.3%, 7 of 30; all J695-treated patients: 34.7%, 52 of 150). The most common infectious AEs reported for any J695 treatment group were nasopharyngitis (12.0%, 18 of 150), upper respiratory tract infection (10.7%, 16 of 150), and bronchitis and viral infection (both 2.7%, 4 of 150). No serious infectious AEs were reported.

Two patients reported malignancies during the study. One placebo-treated patient was diagnosed with ovarian cancer, which was ongoing as of day 129. One J695-treated patient (200 mg×4) was diagnosed with a non-melanoma skin cancer (squamous cell carcinoma) that was removed on day 133. The medical history for this patient included removal of a benign skin growth in March 2005.

There were no clinically significant hematology, chemistry (including blood glucose concentrations), or vital sign changes compared with placebo.

### III. Conclusion

The phase II, multicentre, randomised, double-blind, placebo-controlled trial described in this Example demonstrated statistically and clinically significant efficacy of J695 in the treatment of moderate to severe chronic plaque psoriasis. With the exception of the J695 200 mg×1 treatment group, 90% or more of patients in all J695 treatment groups achieved PASI 75 or greater by week 12, compared with 3.3% of placebo-treated patients. Even in the group that received only 1 dose of study drug (200 mg×1), a majority (63.3%) of patients had achieved at least PASI 75 by week 12. In addition, almost 100% of patients treated with J695 reached PASI 50 or greater, which is considered to be a clinically significant improvement (Carlin C S, Feldman S R, Krueger to J G, Menter A, Krueger G G. J Am Acad Dermatol 2004; 50: 859-66) by week 12. The results for other secondary endpoints, such as PASI 90 and PGA of clear or minimal, were consistent with and supported the primary efficacy analysis.

Response to J695 was rapid. Statistically significant separation between placebo- and J695-treated patients occurred as early as week 1 for the mean percentage improvement in PASI scores Improvement was sustained for the 12-week duration of the trial, even for patients in the J695 200 mg×1 and 200 mg×4 dosage groups.

J695 was well tolerated, and most AEs were mild. Although J695-treated patients were significantly more likely to experience an AE at least possibly related to study drug, most of these were injection site-related AEs (injection-site reaction, erythema, pruritus, or irritation). There was no apparent association between an increased dose of J695 and an increased incidence of AEs. Of note, there were no myocardial or cerebral infarctions.

Immunologic-related events are of particular interest for patients receiving anti-IL-12/23 antibodies. The most frequently reported infectious AEs were nasopharyngitis, upper respiratory tract infection, bronchitis, and viral infection. There were no serious infectious AEs reported for the duration of this trial. Of the 2 malignancies diagnosed during the study, ovarian cancer was diagnosed in a placebo-treated patient, and non-melanoma skin cancer was diagnosed in an J695-treated patient who had a history of a benign skin growth.

In summary, J695 demonstrated statistically and clinically significant benefit for the treatment of patients with moderate to severe chronic plaque psoriasis, and was well tolerated.

### Example 5

### Maintenance of Response with the Fully Human IL-12/-23 Monoclonal Antibody, J695, in the Treatment of Moderate to Severe Plaque Psoriasis

The efficacy and safety of J695 was evaluated in a 12-week, Phase II, randomized controlled trial and 36-week follow-up phase. The objective of the following example was to analyze maintenance of response following discontinuation of therapy during the second 12 weeks of this Phase II study of subcutaneous injections of J695 in the treatment of moderate to severe plaque Ps.

Adults with Ps affecting ≧10% body surface area (BSA) and a PASI score ≧12 were eligible for this 12-week, double-blind, placebo-controlled study. Patients were to randomized to 1 of 6 arms:

1) 100-mg J695 every other week (eow) for 12 wks;

2) one 200-mg J695 dose at wk 0;

3) 200-mg J695 every wk for 4 wks;

4) 200-mg J695 eow for 12 wks;

5) 200-mg J695 every wk for 12 wks; or

6) placebo.

The primary endpoint was a ≧PASI 75 response at Week 12. Patients who met the primary endpoint entered a 36-week blinded/retreatment phase. Treatment with study drug was discontinued, and patients were monitored for PASI score at various times during the 36-week follow-up period, including PASI 50, PASI 75 and PASI 90 responses. Maintenance of PASI response was evaluated through Week 24.

A total of 180 patients enrolled, 30 in each arm. Baseline characteristics were similar between arms (mean values presented except % male): age, 46 years, 74% male; 21 years duration of Ps; PASI=19; and 25% BSA affected.

At Week 12, the percentages of patients with ≧PASI 75 were statistically significantly greater in each of the 5 J695 arms vs. placebo (Table 4). At Week 24, substantial percentages of PASI 75 responders in the active treatments arms had maintained at least a PASI score of ≧PASI 50. Further, substantial percentages of PASI 75 responders in the active treatments arms had also maintained at least a PASI score of ≧PASI 75, as well as a PASI score of ≧PASI 90 (Table 3).

TABLE 3
24-Week Efficacy of J695

| | | Maintenance | Maintenance | Maintenance |
|---|---|---|---|---|
| | | of ≧PASI 50 | of ≧PASI 75 | of ≧PASI 90 |
| | | Response: | Response: | Response: |
| | ≧PASI 75 | Wk 24 vs | Wk 24 vs | Wk 24 vs |
| | at Wk 12 | Wk 12 | Wk 12 | Wk 12 |
| 100 mg eow for | 93%* | 71% | 60% | 33% |
| 12 wks | | | | |
| 200 mg, one dose | 63%* | 68% | 23% | 7% |
| 200-mg every wk | 90%* | 82% | 60% | 23% |
| for 4 wks | | | | |
| 200-mg eow for | 93%* | 89% | 73% | 53% |
| 12 wks | | | | |
| 200-mg every wk | 90%* | 85% | 83% | 57% |
| for 12 wks | | | | |
| Placebo | 3% | — | 7% | 7% |

*p < 0.001 vs. placebo, NRI.

In conclusion, J695 was significantly more efficacious than placebo in the treatment of moderate to severe plaque Ps. Substantial percentages of PASI 75 responders maintained a response of ≧PASI 50, ≧PASI 75, and ≧PASI 90 at Week 24, following discontinuation of active therapy.

### Example 6

### Maintenance of Re-treatment Response with the Fully Human IL-12/-23 Monoclonal Antibody, J695, in the Treatment of Moderate to Severe Plaque Psoriasis

The efficacy and safety of J695 was evaluated in a 48-week, Phase II, randomized controlled trial that included a 12-week initial treatment phase and a 36-week re-treatment phase of patients responding to initial treatment. The initial 12-week efficacy results and maintenance of response results are described in the above examples. The objective of the following example was to examine the re-treatment response during the 36-week re-treatment/follow-up phase in patients who lost their initial responses of this Phase II study of subcutaneous injections of J695 in the treatment of moderate to severe plaque Ps. The further objective of the following example was to examine safety of subcutaneous injections of J695 in the treatment of moderate to severe plaque Ps through 48 weeks.

At baseline, demographics and clinical characteristics were similar across treatment groups. Adults with psoriasis affecting ≧10% body surface area and a Psoriasis Area and Severity Index (PASI) score ≧12 were randomized to 1 of 6 arms: 1) one 200-mg dose J695 at Week 0; 2) 100 mg of J695 every other wk (eow) for 12 weeks; 3) 200 mg of J695 weekly for 4 weeks; 4) 200 mg of J695 eow for 12 weeks; 5) 200 mg of J695 weekly for 12 weeks; or 6) placebo. The primary endpoint was a ≧PASI 75 response at Week 12. Patients who met the primary endpoint entered a 36-week re-treatment phase. Treatment with study drug was discontinued, and patients who lost response (≦PASI 50) during weeks 12-36 received re-treatment with the same dosing regimen assigned during the initial 12-week period. Re-treatment lasted for 12 weeks. Regardless of disposition, all patients were monitored for the entire duration of the study, or until discontinuation.

Of the 180 patients initially enrolled, 130 (1 placebo) entered the retreatment phase and 58 (all J695) were re-treated. The percentages of patients who achieved ≧PASI 75 at week 12 and then again at 12 weeks after re-treatment were as follows for each group: one 200-mg dose, 63% vs. 55%; 100 mg eow, 93% vs 94%; 200 mg weekly 4 wks, 90% vs. 69%; 200 mg eow, 93% vs. 75%; and 200 mg weekly, 90% vs. 83%, respectively. Of the total 58 patients who were retreated, 76% achieved ≧PASI 75 at 12 weeks after re-treatment.

The percentages of patients who achieved ≧PASI 50 at 12 weeks after re-treatment were as follows for each group: one 200-mg dose, 82%; 100 mg eow, 100%; 200 mg weekly 4 wks, 77%; 200 mg eow, 83%; and 200 mg weekly, 100%. Of the total 58 patients who were retreated, 88% achieved ≧PASI 50 at 12 weeks after re-treatment.

The percentages of patients who achieved a PGA of “clear” or “minimal” at 12 weeks after re-treatment were as follows for each group: one 200-mg dose, 36%; 100 mg eow, 75%; 200 mg weekly 4 wks, 62%; 200 mg eow, 67%; and 200 mg weekly, 83%. Of the total 58 patients who were retreated, 64% achieved a PGA of “clear” or “minimal” at 12 weeks after re-treatment.

Adverse events (AEs) occurring ≧5% in at least 1 treatment group in descending order through week 48 were: nasopharyngitis, injection-site reaction, upper respiratory tract infection, headache, hypertension, and arthralgia. The foregoing data demonstrate that J695 was highly efficacious in the treatment of moderate to severe psoriasis. Upon loss of response and re-treatment, a majority of patients were able to re-achieve a PASI 75 response. Moreover, J695 appears to have a favorable safety profile in the long term.

### Example 7

### Pharmacokinetics of a Fully Human IL-12/-23 Monoclonal Antibody, J695, in Normal Healthy Volunteers

The tolerability, safety, and pharmacokinetics (PK) of a range of doses of J695 were evaluated in a randomized, double-blind, placebo-controlled dose-ranging study. The objective of the following example was to investigate the pharmacokinetics of intravenous (IV) and subcutaneous (SC) injections of J695 in healthy volunteers.

The main inclusion criteria were: (i) healthy male volunteers between 18 and 45 years of age; (ii) no clinically relevant abnormalities in any of the investigations of the screening examination (physical exam, vital signs, electrocardiogram, biochemistry, hematology, urinalysis, serology); and (iii) chest x-rays normal within 12 months prior to entering the study. The main exclusion criteria were: (i) smoking more than 10 cigarettes per day; (ii) drinking more than 30 g of alcohol per day; (iii) positive urine drug screen; (iv) chronic infections, especially by intracellular bacterial pathogens such as Mycobacterium tuberculosis; and (v) major infections requiring hospitalization or IV antibiotics within the previous 2 years.

Young (18-45 years of age), healthy male volunteers received 2 equal doses (1 IV and 1 SC administered 8 weeks apart) of 0.1, 0.3, 1.0, or 5.0 mg/kg J695 in a 2-period crossover (2×2 Latin square) design. Blood samples for the determination of J695 concentrations were collected before the first dose (0) and at 0.5, 1, 1.5, 2, 4, 8, 12, 24, 48, 72, 120, 168, 336, 504 and 672 hours after dosing. Serum concentrations of J695 were measured by an enzyme-linked immunosorbent assay.

J695 serum concentrations were tabulated individually, described by statistical characteristics (including geometric mean and geometric standard deviation) and displayed as individual as well as mean, median, and geometric mean concentration vs. time curves for IV and SC treatment and each treatment group. The following PK parameters were estimated using noncompartmental methods:

Cmaxmaximum serum concentration (μg/mL)

Tmax time to reach Cmax(hr)

AUC area under the serum concentration-time curve (μg×hr/mL)

t½ half-life (hr)

CL clearance (mL/hr) (for IV administration)

Vz volume of distribution (mL) (for IV administration)

CL/F apparent CL (mL/hr) (for SC administration)

V/F apparent Vz (mL) (for SC administration)

A total of 64 patients were randomized; 12 received J695 and 4 received placebo for each dose group. J695 appeared to follow bi-exponential kinetics following IV administration, entering the terminal phase approximately 7 days after administration. The mean±SD terminal half-lives for the 0.1-, 0.3-, 1.0-, and 3.0-mg IV doses were 81.2±55.6, 147±73.2, 208±79.2, and 196±55.4 hours, respectively. The mean±SD terminal half-lives for the 0.1-, 0.3-, 1.0-, and 3.0-mg SC doses were 221±103, 161±92.6, 210±90.9, and 208±79.2 hours, respectively. The mean terminal half-life for IV administration ranged from 81.2±55.6 hours to 208±79.2 hours. The mean terminal half-life for SC administration ranged from 161±92.6 hours to 221±103 hours. The overall mean terminal half-life was 8-9 days.

The pharmacokinetics of J695 (maximum concentration of drug [Cₘₐₓ] or area under the curve [AUC]) increased proportionally to dose after both IV and SC to administrations. The volume of distribution ranged from approximately 8-10 L after IV administration to 24-67 L after SC administration. After SC administration, the time to reach Cₘₐₓ was approximately 3-4 days. Bioavailability after SC administration ranged between 42% and 62% for the doses evaluated. The pharmacokinetic parameters following IV or SC administration at each dose, including Cₘₐₓ (the maximum serum concentration in μg/mL), AUC (area under the serum concentration-time curve in μg×hr/mL), tₘₐₓ (time to reach Cₘₐₓin hrs), t_{1/2} (half-life in hrs), CL (clearance in mL/hr) and Vz (volume of distribution (mL)), are displayed in Table 4 below.

TABLE 4
PK Parameters (Mean ± SD) in Healthy Human Volunteers
Following IV or SC Administration of J695

| Cohort | | Cₘₐₓ | | AUC_{0-∞} | | | |
|---|---|---|---|---|---|---|---|
| (mg/kg) | Route | (μg/ml) | tₘₐₓ (hr) | (μg × hr/ml) | t_{1/2} (hr) | C_{L}* | Vz† |
| 0.1 | IV | 1.99 ± 0.931 | — | 146 ± 78.8 | 81.2 ± 55.6 | 596 ± 1850 | 8010 ± 7600 |
| 0.3 | IV | 7.99 ± 3.08 | — | 562 ± 202 | 147 ± 73.2 | 50.4 ± 32.7 | 8512 ± 3746 |
| 1.0 | IV | 27.7 ± 8.33 | — | 2410 ± 717 | 208 ± 79.2 | 36.2 ± 9.80 | 10400 ± 3840 |
| 5.0 | IV | 150 ± 50.6 | — | 12700 ± 3390 | 196 ± 55.4 | 33.6 ± 9.26 | 9360 ± 3360 |
| 0.1 | SC | 0.245 ± 0.100 | 66.7 ± 10.6 | 84.4 ± 40.6 | 221 ± 103 | 183 ± 248 | 66500 ± 135000 |
| 0.3 | SC | 1.09 ± 1.12 | 90.0 ± 43.6 | 244 ± 150 | 161 ± 92.6 | 183 ± 196 | 24800 ± 7430 |
| 1.0 | SC | 2.83 ± 0.633 | 82.0 ± 23.9 | 1000 ± 318 | 210 ± 90.9 | 91.1 ± 41.2 | 23900 ± 8590 |
| 5.0 | SC | 13.4 ± 5.34 | 82.0 ± 36.1 | 4840 ± 2420 | 208 ± 79.2 | 229 ± 480 | 31800 ± 19500 |

*For SC administration, CL/F
†For SC administration, V/F

The foregoing data demonstrate that J695 administered IV and SC in single doses between 0.1 and 5.0 mg/kg was well-tolerated by young healthy male individuals. The pharmacokinetic properties of J695, with its half-life of 8-9 days, are as would be expected for an IgG₁ antibody.

### Example 8

### Maintenance of Re-treatment Response with the Fully Human IL-12/-23 Monoclonal Antibody, J695, in the Treatment of Moderate to Severe Plaque Psoriasis

The efficacy and safety of J695 was evaluated in a 48-week, Phase II, to randomized controlled trial that included a 12-week initial treatment phase and a 36-week re-treatment phase of patients responding to initial treatment. The initial 12-week efficacy results and maintenance of response results are described in examples 1-5 above. The objective of the following example was to examine the re-treatment response during the 36-week re-treatment/follow-up phase in patients who lost their initial responses of this Phase II study of subcutaneous injections of J695 in the treatment of moderate to severe plaque Ps. The further objective of the following example was to examine safety of subcutaneous injections of J695 in the treatment of moderate to severe plaque Ps through 48 weeks.

The main inclusion criteria for the trial were: (i) adults with clinical diagnosis of psoriasis for at least 6 months and stable plaque psoriasis for at least 2 months prior to screening; and (ii) moderate to severe plaque psoriasis (≧10% body surface area involvement, Psoriasis Area and Severity Index [PASI] score ≧12 and a Physician's Global Assessment [PGA] of at least moderate disease) at the baseline visit.

A first exclusion criteria for the trial was previous exposure to systemic or biologic anti-IL-12 therapy. A second exclusion criteria was inability to discontinue the following therapies before the baseline visit: topical psoriasis therapies >2 weeks prior; ultraviolet (UV)-B light phototherapy ≧2 weeks prior; psoralen-UV light phototherapy ≧4 weeks prior; systemic therapies ≧4 weeks prior; and biologic therapies ≧12 weeks prior.

At baseline, demographics and clinical characteristics were similar across treatment groups.

Adults with psoriasis affecting ≧10% body surface area and a Psoriasis Area and Severity Index (PASI) score ≧12 were randomized to 1 of 6 arms: 1) one 200-mg dose J695 at Week 0; 2) 100 mg of J695 every other wk (eow) for 12 weeks; 3) 200 mg of J695 weekly for 4 weeks; 4) 200 mg of J695 eow for 12 weeks; 5) 200 mg of J695 weekly for 12 weeks; or 6) placebo. The primary endpoint was a ≧PASI 75 response at Week 12. Patients who met the primary endpoint entered a 36-week re-treatment phase. Treatment with study drug was discontinued, and patients who lost response (≦PASI 50) during weeks 12-36 received re-treatment with the same dosing regimen assigned during the initial 12-week period. Re-treatment lasted for 12 weeks. Regardless of disposition, all patients were monitored for the entire duration of the study, or until discontinuation.

Outcome measurements included the following: (i) percentage of patients achieving PASI 75; (i) median time to achieve PASI 75 response after retreatment; (iii) median time to lose PASI 75 response (iii) percentage of patients with a PGA score of “Clear” or “Minimal” after retreatment.

Statistical analysis was carried out as follows. Intention-to-treat (ITT) analyses were performed by randomized treatment group. For PASI assessments obtained after retreatment with J695, the assessments were assigned to study visits according to the number of days after the first dose of the retreatment. The proportion of patients achieving PASI response (yes/no) are presented according to the derived study visit. All statistical tests were 2-tailed with a significance value of 0.05

Of the 180 patients initially enrolled (30 patients per treatment group), 130 (1 placebo) entered the retreatment phase and 58 (all J695) were re-treated. The percentages of patients who achieved ≧PASI 75 at week 12 and then again at 12 weeks after re-treatment were as follows for each group: one 200-mg dose, 63% vs. 55%; 100 mg eow, 93% vs 94%; 200 mg weekly 4 wks, 90% vs. 69%; 200 mg eow, 93% vs. 75%; and 200 mg weekly, 90% vs. 83%, respectively. Of the total 58 patients who were retreated, 76% achieved ≧PASI 75 at 12 weeks after re-treatment. A majority of patients were able to re-achieve a PASI 75 response.

The median time to achieve ≧PASI 75 during retreatment were as follows for each group: one 200-mg dose, between 60 and 65 days; 100 mg eow, between 55 and 60 days; 200 mg weekly 4 wks, between 55 and 60 days; 200 mg eow, between 25 and 35 days; and 200 mg weekly, between 55 and 60 days, respectively.

The median time to lose PASI 75 following the initial 12 weeks of treatment were as follows for each group: one 200-mg dose, between 55 and 60 days; 100 mg eow, between 110 and 120 days; 200 mg weekly 4 wks, between 110 and 120 days; 200 mg eow, between 160 and 180 days; and 200 mg weekly, between 180 and 190 days, respectively.

The percentages of patients who achieved a PGA of 0 or 1 during re-treatment were as follows for each group: one 200-mg dose, between 35% and 40%; 100 mg eow, between 70% and 80%; 200 mg weekly 4 wks, between 60% and 65%; 200 mg eow, between 60% and 70%; and 200 mg weekly, between 80% and 90%, respectively. Of the total patients who were retreated, between 60 and 65% achieved a PGA of 0 or 1 after re-treatment.

Adverse events (AEs) occurring ≧5% in at least 1 treatment group in descending order through week 48 were: nasopharyngitis, injection-site reaction, upper respiratory tract infection, headache, hypertension, and arthralgia.

The foregoing data demonstrate that J695 was highly efficacious in the treatment of moderate to severe psoriasis. Upon loss of response and re-treatment, a majority of patients were able to re-achieve a PASI 75 response. Moreover, J695 appears to have a favorable safety profile in the long term.

### Example 9

### Population Pharmacokinetics of Briakinumab in Subjects with Moderate to Severe Plaque Psoriasis

Briakinumab (J695) is a monoclonal antibody directed against the shared p40 subunit of IL-12/23. Briakinumab demonstrated a high rate of efficacy in the treatment of moderate to severe plaque psoriasis in one Phase 2 study, and four Phase 3 studies. Following single dose SC and IV injections, briakinumab has a mean half-life of approximately 8 days.

The population pharmacokinetics (Pop PK) of briakinumab were characterized using data from one Phase 1 study in healthy volunteers, and one Phase 2 and four Phase 3 studies in subjects with moderate to severe plaque psoriasis. The Pop PK model described the relationships between briakinumab serum concentration-time data and subject-specific covariates to explain variability. The model was built using nonlinear mixed effect modeling (NONMEM) based on NONMEM VI with the Intel Fortran compiler (Version 9). The first-order conditional estimation method (FOCE) with INTERACTION (FOCEI) was employed within NONMEM. Briakinumab pharmacokinetics were described as a two compartment model. Clearance (C_{L}) and volume of distribution in the central compartment (V_{c}) estimates were 0.779 L/day and 6.04 L, with interindividual variabilities of 7.0% and 16.9%, respectively.

Statistically significant effects of body weight, gender, and presence of binding and neutralizing anti-drug antibodies on C_{L}, race on V_{c}, and weight on volume of distribution of peripheral compartment (Vₚ) were found. Other tested covariates did not statistically significantly affect these parameters. A power function was found to be most appropriate for the residual error model. Model evaluation including bootstrapping and visual predictive checks suggested that the final pharmacokinetic model was robust.

### Dataset and Analysis Conventions

Briakinumab serum concentration data were included from all subjects who received at least one dose of briakinumab and had at least one measurable briakinumab concentration above the lower limit of quantitation (LLOQ; 15.63 ng/mL) Subjects included were from four Phase 3 studies, one Phase 2, and one Phase 1 study as shown in the table below.

For the Phase 1 study, data from subjects administered a single 100 mg SC or IV injection of briakinumab were included. Data from 1624 subjects were included in the population pharmacokinetic analyses. Serum concentrations that were below LLOQ during active treatment were set to LLOQ/2. The first concentration LLOQ after the last recorded dose was set at LLOQ/2, and all subsequent LLOQ concentrations were excluded.

TABLE 5
Studies included in the pharmacokinetic analysis

| Study | Treatment Duration | Briakinumab Treatments |
|---|---|---|
| M05-736# | 12 weeks double blind | 100 mg SC every other week |
| | and 36 weeks | (eow) for 12 weeks |
| | observation/re | 200 mg SC one dose at Week 0 |
| | treatment | 200 mg SC every week for |
| | | 4 weeks |
| | | 200 mg SC eow for 12 weeks |
| | | 200 mg SC every week for |
| | | 12 weeks |
| M06-890* | 52 weeks double-blind | 200 mg SC at Week 0, 4, 100 mg |
| | | at Week 8; |
| | | 100 mg SC Q4 starting at Week 12 |
| | | 100 mg SC Q12 starting at |
| | | Week 12 |
| | | 200 mg SC at Weeks 0, 4, 100 mg |
| | | at Week 8 |
| M10-114*, | 12 weeks double-blind | 200 mg SC at Weeks 0, 4, 100 mg |
| M10-315* | | at Week 8 |
| M10-255* | 52 weeks double-blind | 200 mg SC at Week 0 and Week 4, |
| | | 100 mg SC Q4 starting at Week 8 |
| M10-220** | Single dose healthy | Single 100 mg SC injection |
| | volunteers | Single 100 mg IV injection |

#Phase 2,
*Phase 3,
**Phase 1.

### Model Building

The Pop PK model was built using nonlinear mixed effect modeling (NONMEM) based on NONMEM VI compiled with the Intel Fortran compiler (Version 9). The first-order conditional estimation method (FOCE) with INTERACTION (FOCEI) was employed. Modeling was conducted in a stepwise manner: development of an structural model, followed by addition of models for interindividual variability (IIV) and residual error and covariate testing IIV was modeled using either exponential or additive error models. Residual variability was modeled using additive, proportional, to a combination of additive and proportional, or power function error models. Parameter estimates from the final model were tested for robustness by bootstrap validation, goodness-of-fit plots, visual and numeric predictive checks.

Selection of model was based on the following: more random distribution of observed and predicted serum concentrations across the line of unity, weighted residuals having less systematic bias, and adequate goodness of fit plots, physiologically reasonable and/or statistically significant estimates of mean parameters and their standard errors. Likelihood ratio test was used for hypothesis testing to discriminate between alternative models. For forward inclusion, the reduction of Objective Function Value (OFV) of at least 6.63 and 9.21 for one and two degrees of freedom, respectively (significance level of P<0.01). For backward elimination, changes in OFV were 10.83 and 13.82 for one and two degrees of freedom, respectively (significance level of 0.001).

### Covariates Analysis

Covariates were investigated using forward/backward elimination at a significance level of P<0.001. Continuous covariates were entered in a linear function, while dichotomous covariates were entered as binary indicator variables. Covariates were tested in a stepwise procedure. If a significant covariate was identified, it was included in the starting model for the next iteration of the model building process. The covariates investigated included: Baseline Demographics (e.g age, sex, race, body weight [WGT], etc.), Baseline Laboratory Measures (e.g. AST, ALT, serum creatinine, etc.), and other patient specific factors (presence of anti-drug antibodies (ADA), and presence of neutralizing ADA, etc.).

TABLE 6
Demographics of Subjects Treated with Briakinumab

| Demographic Characteristic | Briakinumab Treated (N = 1624) |
|---|---|
| Sex, N (%) | |
| Male | 1107 (68.2%) |
| Female | 517 (31.8%) |
| Race, N (%) | |
| White | 1468 (90.39%) |
| Black | 59 (3.63%) |
| Other | 97 (5.97%) |
| Age (years) | |
| Mean (SD) | 44.7 (13.57) |
| Range | 18-93 |
| Weight (kg) | |
| Mean (SD) | 92.2 (22.83) |
| Range | 43.0-209.0 |

### Results

Subjects administered briakinumab were primarily Caucasian males with mean age of 44 years and mean weight of 92 kg (Table 6) The Pop PK of briakinumab was described as a two-compartment model with two exponential terms for inter-individual variability on C_{L} and V_{c}. (FIG. 1) Absorption from the site of SC administration was described by Kₐ and absolute bioavailability represented by F1.

The model adequately described the observations over the entire briakinumab serum concentration range (FIG. 2). Observed and predicted briakinumab concentrations were randomly distributed across the line of unity. There were no systemic trends with time. Data at higher concentrations were more sparse, with a slight trend to underprediction of briakinumab concentrations. The estimated central values for briakinumab CL and Vc were 0.779 L/day and 6.04 L, respectively (Table 7)

Inter individual variabilities for C_{L} and V_{c} were 8.8% and 12.9%, respectively. A power function was found to be most appropriate for the residual error model C_{L} increased with increasing bodyweight and in the presence of ADA and neutralizing ADA. With increasing weight, C_{L} increased approximately 10% per 10 kg change from <75 kg to 105 kg, then approximately 7.5% when weight was >105 kg. Overall, the presence of ADA and neutralizing ADA increased the CL of briakinumab by approximately 30% and 66%, respectively. The covariate model for Vc included race, where Vc was approximately 30% greater in Caucasian subjects than those subjects from other races. This finding can be attributed to bodyweight, as Vc estimates in Caucasian and non-Caucasian subjects were similar when adjusted for bodyweight.

TABLE 7
Parameter Estimates and Variability for the Final Briakinumab
Population Pharmacokinetic Model

| Parameter | Population Estimate (SEE) | % RSE |
|---|---|---|
| CL (L/day) | 0.779 (0.0683) | 8.77 |
| kₐ (1/day) | 0.614 (0.0697) | 11.4 |
| Vc (L) | 6.04 (0.779) | 12.9 |
| V2 (L) | 3.18 (0.304) | 9.56 |
| Q (L/day) | 0.805 (0.101) | 12.5 |
| Bioavailability (F1) | 0.392 (0.0368) | 9.39 |
| Exponent in Residual Error Model | 0.783 (0.0111) | 1.42 |
| CL - Inter-Ind. Variab. (% CV)* | 0.181 (42.5)* | 7.02 |
| Vc - Inter-Ind. Variab. (% CV)* | 0.827 (90.9)* | 16.9 |
| Residual Error | 0.147 (0.00476) | 3.24 |

SEE = Standard Error of Estimate;
% RSE, SEE/population estimate * 100,
% CV = SQRT(ETA) * 100

### Conclusions

A population PK model was developed that adequately described the pharmacokinetics of briakinumab in subjects with moderate to severe plaque psoriasis. The pharmacokinetics of briakinumab were described using a two-compartment model. The estimated central values for briakinumab C_{L} and V_{c} were 0.779 L/day and 6.04 L, respectively. Inter individual variabilities for C_{L} and V_{c} were 8.8% and 12.9%, respectively. A power function was found to be most appropriate for the residual error model. Significant covariates on C_{L} included bodyweight and in the presence of ADA and neutralizing ADA, which increased the C_{L} in subjects. Overall, the presence of ADA and presence of neutralizing ADA increased the C_{L} of briakinumab by approximately 30% and 66%, respectively. The covariate model for V_{c} included race, where V_{c} was approximately 30% greater in Caucasian subjects, which can be attributed to body weight.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. An isolated antibody, or antigen-binding portion thereof, which is capable of binding to an epitope of the p40 subunit of IL-12 and/or IL-23, wherein the antibody, or antigen binding portion thereof, when administered subcutaneously or intravenously to a subject at a dose of about 100 mg or about 200 mg, is capable of exhibiting one or more pharmacokinetic properties selected from the group consisting of:
a) a rate of clearance (C_{L}) of about 0.5 to about 1.0 L/day;
b) an absorption constant (kₐ) of about 0.4 to about 0.8 L/day;
c) a volume of central compartment volume (V_{c}) of about 3.5 to about 8.5 L;
d) a second (peripheral compartment) volume (V₂) of about 2.2 to about 4.2 L;
e) a rate of clearance from the central compartment to the second compartment (Q) of about 0.6 to about 1.1 L/day; and
f) a bioavailability (F1) of about 0.29 to about 0.50.

2. The isolated antibody, or antigen-binding portion thereof, of claim 1, wherein the antibody, or antigen binding portion thereof, has 1, 2, 3, 4, 5, or 6 of the pharmacokinetic properties of claim 1.

3. The isolated antibody, or antigen-binding portion thereof, of claim 1, wherein the antibody, or antigen-binding portion thereof, exhibits a rate of clearance (C_{L}) of about 0.5 to about 1.0 L/day.

4. The isolated antibody, or antigen-binding portion thereof, of claim 3, wherein the antibody, or antigen-binding portion thereof, exhibits a rate of clearance (C_{L}) of about 0.8 L/day.

5. The isolated antibody, or antigen-binding portion thereof, of claim 1, wherein the antibody, or antigen-binding portion thereof, exhibits an absorption constant (kₐ) of about 0.4 to about 0.8 L/day.

6. The isolated antibody, or antigen-binding portion thereof, of claim 5, wherein the antibody, or antigen-binding portion thereof, exhibits an absorption constant (kₐ) of about 0.6 L/day.

7. The isolated antibody, or antigen-binding portion thereof, of claim 1, wherein the antibody, or antigen-binding portion thereof, exhibits a volume of central compartment volume (V_{c}) of about 3.5 to about 8.5 L.

8. The isolated antibody, or antigen-binding portion thereof, of claim 7, wherein the antibody, or antigen-binding portion thereof, exhibits a volume of central compartment volume (V_{c}) of about 6.0 L.

9. The isolated antibody, or antigen-binding portion thereof, of claim 1, wherein the antibody, or antigen-binding portion thereof, exhibits a second (peripheral compartment) volume (V₂) of about 2.2 to about 4.2 L.

10. The isolated antibody, or antigen-binding portion thereof, of claim 9, wherein the antibody, or antigen-binding portion thereof, exhibits a second (peripheral compartment) volume (V₂) of about 3.2 L.

11. The isolated antibody, or antigen-binding portion thereof, of claim 1, wherein the antibody, or antigen-binding portion thereof, exhibits a rate of clearance from the central compartment to the second compartment (Q) of about 0.6 to about 1.1 L/day.

12. The isolated antibody, or antigen-binding portion thereof, of claim 11, wherein the antibody, or antigen-binding portion thereof, exhibits a rate of clearance from the central compartment to the second compartment (Q) of about 0.8 L/day.

13. The isolated antibody, or antigen-binding portion thereof, of claim 1, wherein the antibody, or antigen-binding portion thereof, exhibits a bioavailability (F1) of about 0.29 to about 0.50.

14. The isolated antibody, or antigen-binding portion thereof, of claim 13, wherein the antibody, or antigen-binding portion thereof, exhibits a bioavailability (F1) of about 0.4.

15. The isolated antibody, or antigen-binding portion thereof, of claim 1, wherein the antibody is administered intravenously.

16. The isolated antibody, or antigen-binding portion thereof, of claim 1, wherein the antibody is administered subcutaneously.

17. The isolated antibody, or antigen-binding portion thereof, of claim 1, wherein the antibody has been administered once.

18. The isolated antibody, or antigen-binding portion thereof, of claim 1, wherein the antibody has been administered more than once.

19. The isolated antibody, or antigen-binding portion thereof, of claim 1, wherein the antibody, or antigen-binding portion thereof, is administered at a dose of about 100 mg.

20. The isolated antibody, or antigen-binding portion thereof, of claim 1, wherein the antibody, or antigen-binding portion thereof, is administered at a dose of about 200 mg.

21. The isolated antibody, or antigen-binding portion thereof, of claim 1, wherein said pharmacokinetic properties are determined using a two compartment model.

22. The isolated antibody, or antigen-binding portion thereof, of claim 1, wherein the subject is suffering from a disorder in which the activity of the p40 subunit of IL-12 and/or IL-23 is detrimental.

23. The isolated antibody, or antigen-binding portion thereof, of claim 1, wherein the antibody is J695.

24. A method for inhibiting the activity of the p40 subunit of IL-12 and/or IL-23 in a subject suffering from a disorder in which the activity of the p40 subunit of IL-12 and/or IL-23 is detrimental, comprising administering to the subject the isolated antibody, or antibody binding portion thereof, of claim 1, such that the activity of the p40 subunit of IL-12 and/or IL-23 in the subject is inhibited.

25. A method for treating a subject suffering from a disorder in which the activity of the p40 subunit of IL-12 and/or IL-23 is detrimental, comprising administering to the subject an antibody, or antigen-binding portion thereof, of claim 1, thereby treating the subject.

26. The method of claim 24 or 25, wherein the disorder in which the activity of the p40 subunit IL-12 and/or IL-23 is detrimental is psoriasis.

27. The method of claim 26, wherein the psoriasis is moderate to severe plaque psoriasis.

28. The method of claim 24 or 25, further comprising the administration of an additional agent.

29. A pharmaceutical composition comprising an antibody, or antigen-binding portion thereof, which is capable of binding to an epitope of the p40 subunit of IL-12 and/or IL-23, wherein the pharmaceutical composition, when administered subcutaneously or intravenously to a subject at a dose of about 100 mg or about 200 mg, allows said antibody, or antigen-binding portion thereof, to exhibit one or more pharmacokinetic properties selected from the group consisting of:
a) a rate of clearance (C_{L}) of about 0.5 to about 1.0 L/day;
b) an absorption constant (kₐ) of about 0.4 to about 0.8 L/day;
c) a volume of central compartment volume (V_{c}) of about 3.5 to about 8.5 L;
d) a second (peripheral compartment) volume (V₂) of about 2.2 to about 4.2 L;
e) a rate of clearance from the central compartment to the second compartment (Q) of about 0.6 to about 1.1 L/day; and
f) a bioavailability (F1) of about 0.29 to about 0.50.

30. The pharmaceutical composition of claim 29, wherein the antibody, or antigen binding portion thereof, has 1, 2, 3, 4, 5, or 6 of the pharmacokinetic properties of claim 29.

31. The pharmaceutical composition of claim 29, wherein the composition is administered intravenously.

32. The pharmaceutical composition of claim 29, wherein the composition is administered subcutaneously.

33. The pharmaceutical composition of claim 29, wherein the composition is administered once.

34. The pharmaceutical composition of claim 29, wherein the composition is administered more than once.

35. The pharmaceutical composition of claim 29, wherein the composition is administered at a dose of about 100 mg.

36. The pharmaceutical composition of claim 29, wherein the composition is administered at a dose of about 200 mg.

37. The pharmaceutical composition of claim 29, wherein said pharmacokinetic properties are determined using a two compartment model.

38. The pharmaceutical composition of claim 29, wherein the subject is suffering from a disorder in which the activity of the p40 subunit of IL-12 and/or IL-23 is detrimental.

39. A method for inhibiting the activity of the p40 subunit of IL-12 and/or IL-23 in a subject suffering from a disorder in which the activity of the p40 subunit of IL-12 and/or IL-23 is detrimental, comprising administering to the subject the pharmaceutical composition of claim 29, such that the activity of the p40 subunit of IL-12 and/or IL-23 in the subject is inhibited.

40. A method for treating a subject suffering from a disorder in which the activity of the p40 subunit of IL-12 and/or IL-23 is detrimental, comprising administering to the subject the pharmaceutical composition of claim 29, thereby treating the subject.

41. The method of claim 39 or 40, wherein the disorder in which the activity of the p40 subunit IL-12 and/or IL-23 is detrimental is psoriasis.

42. The method of claim 41, wherein the psoriasis is moderate to severe plaque psoriasis.

43. The method of claim 39 or 40, further comprising the administration of an additional agent.
